(19) United States
(12) Patent Application Publication
(10) Pub. No.: US 2024/0400606 A1
(43) Pub. Date: Dec. 5, 2024

(54) SMALL MOLECULE ACTIVATORS OF THE IMMUNE SYSTEM

(71) Applicant: THE BOARD OF TRUSTEES OF THE UNIVERSITY OF ILLINOIS, Urbana, IL (US)

(72) Inventors: Paul J. HERGENROTHER, Champaign, IL (US); Lindsay CHATKEWITZ, Newton, MA (US)

(73) Assignee: THE BOARD OF TRUSTEES OF THE UNIVERSITY OF ILLINOIS, Urbana, IL (US)

(21) Appl. No.: 18/701,593

(22) PCT Filed: Oct. 21, 2022
(86) PCT No.: PCT/US2022/078484

§ 371 Date: Apr. 15, 2024

Related U.S. Application Data
(60) Provisional application No. 63/270,221, filed on Oct. 21, 2021.

(51) Int. Cl. C07J 73/00; A61K 31/58; A61P 35/00

(52) U.S. Cl. CPC C07J 73/005; A61K 31/58; A61P 35/00

(57) ABSTRACT

The technology disclosed herein provides (4aR,4bS,6aS,9aS,9bS)-1-(4-chlorobenzyl)-4a,6a-dimethyl-3,4,4a,6,6a,8,9,9a,9b,10-decahydro-1H-indeno[5,4-f]quinoline-2,5,7(4bH)-trione, identified as compound IB:10:D, and related compounds. Compound IB:10:D was discovered though screening a compound library for inhibitors of the enzyme SULT2B1b. This enzyme, which produces the product cholesterol sulfate, is overexpressed in cancer. The product acts as an immunosuppressant that results in suppression of the immune system's ability to clear tumor cells. Compounds related to IB:10:D were synthesized and shown to inhibit production of cholesterol sulfate. The discovered inhibitors of SULT2B1b provide new compounds and methods for immunotherapy and cancer treatment.

### RELATED APPLICATIONS

This application claims priority under 35 U.S.C. § 119(e) (or is related to) to U.S. Provisional Patent Application No. 63/270,221, filed Oct. 21, 2021, which is incorporated herein by reference.

### BACKGROUND OF THE INVENTION

Anticancer immunotherapy, especially immune checkpoint blockade (ICB), has become an extremely important treatment option for oncology, generating high levels of excitement with numerous examples of durable, complete clinical responses. However, it is estimated that only ˜12% of patients that are eligible for ICB would demonstrate a clinical response. Thus, many patients do not gain a therapeutic benefit from immunotherapy and there has been much interest in studying why certain tumors respond while others do not. Many have described the importance of tumor mutational burden (TMB) as a major biomarker. However, TMB is not wholly predictive and there are many immunosuppressive mechanisms cancers employ to evade immune system recognition and clearance. These mechanisms have been well studied. The current overarching hypothesis is that when a cancer-specific immunosuppressive mechanism is perturbed, often through inhibition, the native immune system tumor recognition and cell killing can be activated, a concept validated by ICB strategies.

There are preclinical small molecules and clinical candidates that have been investigated for overcoming the immunosuppressive nature of the tumor microenvironment. Interestingly, many of these drugs target proteins expressed in immune cells, leading to a generalized activation of the whole immune system that has implications for causing long term inflammation and other undesirable effects. An alternative approach is to target proteins that are only present and/or overexpressed in tumor cells, which affords an opportunity to directly modulate the tumor immune microenvironment. This cancer-targeted strategy may avoid potential harmful effects seen with systemic immune system activation, e.g., IL-2 or anti-CTLA4. The validity of this strategy can be seen with the clinical development of indoleamine-2,3-dioxygenase (IDO) and tryptophan-2,3-dioxygenase (TDO) inhibitors which target cancers' overexpression of these enzymes that synthesize immunosuppressive metabolites. Unfortunately, IDO/TDO inhibitors failed in Phase III clinical trials, hypothesized to be due to a variety of factors including potential expression of compensatory enzymes and incomplete inhibition of the target.

As seen in IDO/TDO inhibitor development, metabolites are powerful molecules that lead to an immunosuppressive environment in many tumor types. One such metabolite is cholesterol sulfate (CS) which was initially demonstrated to be found at elevated levels in cancerous tissues. It was not until 2016 when Davis and coworkers (Nat Immunol 2016, 17 (7), 844-50) identified the immunosuppressive activity of CS. The authors showed that CS treatment led to poor activation capacity of T cells (as measured by decreased IL-2 production). More recent work has identified CS as an inhibitor of the leukocyte-specific guanine nucleotide exchange factor, dedicator of cytokinesis 2 (DOCK2). Upon inhibition of DOCK2, the GTPase Ras-related C3 botulinum toxin substrate 1 (RAC1) remains in the inactive, GDP-bound state leading to decreased T cell motility. Since T cells are thought to be major effectors of anticancer immunity and immune cell evasion, this action of CS inhibiting T cell mobility and activation is hypothesized to be the causative mechanism for CS-mediated immunosuppression.

Accordingly, there is a need to further explore cancer-specific immune system normalization with new therapeutic targets and chemical probes. Advances utilizing small molecules to combat immunosuppression would allow access to novel targets.

### SUMMARY

Environments with high levels of cholesterol sulfate have been shown to decrease T cell migration and activation, ultimately decreasing the ability for T cells to function. Lowering levels of cholesterol sulfate through inhibition of SULT2B1b can restore T cell activity, increasing immune clearance of tumor cells and potentially synergizing with other immunotherapies.

Herein, new small molecule inhibitors of SULT2B1b have been identified. Using a series of biochemical and whole cell in vitro experiments and screening of a complexity-to-diversity library of complex molecules (Nat. Chem. 2013, 5, 195-202), compounds that inhibit SULT2B1b in vitro, inhibit whole cell CS production, and modulate CS levels in an in vivo tumor model were discovered. Compounds discussed herein are important chemical probes for validating SULT2B1b as a target for enhancing antitumor immunity and can be used to further understand the interplay between CS and the immune system.

Accordingly, this disclosure provides a compound of Formula I.

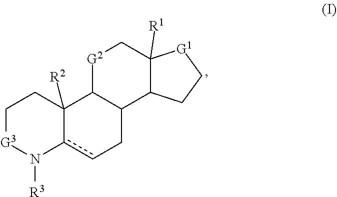

or a pharmaceutically acceptable salt thereof;
wherein
[symbol] is an unsaturated or saturated bond;
G¹ is C═O, CH₂, CHF, CF₂, CHJ¹R^{a}, or C(OCH₂)₂;
   J¹ is O, S, NR^{c}, C(O)X¹ wherein X¹ is O or NR^{d};
G² is C═O, CH₂, CHF, CF₂, CHJ²R^{b}, or C(OCH₂)₂;
   J² is O, S, NR^{g}, C(O)X² wherein X² is O or NR^{h};
G³ is C═O or CH₂;
R^{a}, R^{b}, R^{c}, R^{d}, R^{g}, and R^{h} are each independently H, or -(C₁-C₆)alkyl;
R¹ is -(C₁-C₆)alkyl;
R² is -(C₁-C₆)alkyl;
R³ is —CH₂R⁴, —CH(CH₃)R⁴, R⁴, —C(O)R⁴, or H wherein R³ is not —C(O)R⁴ when G³ is C═O; and
R⁴ is aryl, heteroaryl, cycloalkyl, heterocyclyl, or -(C₁-C₆)alkyl, wherein aryl, heteroaryl, cycloalkyl, and heterocyclyl are each optionally substituted with one or more substituents;
wherein each -(C₁-C₆)alkyl is independently saturated or unsaturated, and optionally substituted with one or more substituents.

In certain preferred embodiments, R³ is:

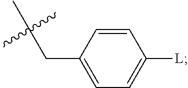

wherein
L is halo, X³R^{e}, or -(C₁-C₆)alkyl;
X³ is O, S, or NR^{f}; and
   R^{e} and R^{f} are H or -(C₁-C₆)alkyl.

This disclosure also provides a method for treatment of cancer comprising, administering to a subject in need of cancer treatment a compound of Formula I wherein the compound increases T-cell activity via inhibition of the enzyme SULT2B1b, wherein immune clearance of the cancer increases in the subject, thereby treating the cancer.

The invention provides novel compounds of Formulas I to V, intermediates for the synthesis of compounds of Formulas I to V, as well as methods of preparing compounds of Formulas I to V. The invention also provides compounds of Formulas I to V that are useful as intermediates for the synthesis of other useful compounds. The invention provides for the use of compounds of Formulas I to V for the manufacture of medicaments useful for the treatment of cancer in a mammal, such as a human.

The invention provides for the use of the compositions described herein for use in medical therapy. The medical therapy can be treating cancer, for example, breast cancer, liver cancer, lung cancer, pancreatic cancer, prostate cancer, or colon cancer. The invention also provides for the use of a composition as described herein for the manufacture of a medicament to treat a disease in a mammal, for example, cancer in a human. The medicament can include a pharmaceutically acceptable diluent, excipient, or carrier.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1. (A) Results obtained by performing the coupled chromogenic in vitro assay using recommended conditions in the presence or absence of DHEA. Data plotted as mean s.e.m.; n=3 independent replicates. (B) Results obtained from the coupled chromogenic in vitro assay in the presence or absence of DHEA after assay optimization (conditions reported in methods). Data plotted as mean±s.e.m.; n=3 independent replicates. Statistics were determined with a two-tailed student t-test; ns: not significant, *p<0.05, **p<0.01, ***p<00.001.

FIG. 2. In vitro IC₅₀ determination using the coupled chromogenic in vitro assay with optimized conditions. Data plotted as mean±s.e.m.; n=3 independent replicates.

FIG. 3. IB:10:D (1) inhibits SULT2B1b in vitro. An LC-MS/MS based in vitro assay was used for analysis. SULT2B1b, PAPS, DHEA, and compound of interest were incubated for 2 hours, then inhibition of SULT2B1b was determined by quantification of DHEA-sulfate formation in the presence or absence of compound of interest. (A) IMAPD1 inhibition by IB:10:D was assessed using an IMPAD1 in vitro activity assay where IB:10:D, PAP, and IMPAD1 were incubated, and inorganic phosphate formation was measure using a malachite green phosphate detection solution (analogous to second half of coupled chromogenic in vitro assay in Scheme 1). Data plotted as mean±s.e.m.; n=3 independent replicates. (B) hCAII inhibition by IB:10:D was assessed using an in vitro hCAII activity assay. hCAII and 4-nitrophenyl acetate were incubated in the presence of DMSO or IB:10:D and production of 4-nitrophenol was monitored by measuring absorbance at 348 nm. Data plotted as mean±s.e.m.; n=3 independent replicates. (C) Inhibition of SULT2B1b by IB:10:D due to an aggregation-type mechanism was measured by inclusion of 0.1 mg/mL bovine serum albumin (BSA) in the LC-MS/MS based in vitro assay. Congo red is used as a positive control. Data plotted as mean±s.e.m.; n=3 biological replicates. Statistics were determined with a two-tailed student t-test; ns: not significant, *p<0.05, **p<0.01, ***p<00.001.

FIG. 4. SULT2B1b is overexpressed in a variety of cancer cell lines. SULT2B1b expression across cell lines as visualized by western blot.

FIG. 5. Expression levels of SULT2B1b and STS play a crucial role in governing whole cell CS levels. (A) CS levels in cell lines with high and low expression of SULT2B1b were analyzed. 1.5×10⁶ cells of each cell line were lysed and submitted to LC-MS/MS analysis for CS quantification. Data plotted as mean±s.e.m.; n=3 independent replicates. (B) Relative basal expression levels of SULT2B1b and STS were visualized by western blot across cell lines. Steroid sulfatase overexpression lysate and empty vector lysate are used as positive and negative controls, respectively. Blot is a representative image of two independent replicates. (C) Quantification of relative expression levels of SULT2B1b and STS revealed a correlation between the ratio of SULT2B1b/STS and whole cell CS levels. Quantification was calculated using ImageJ. Data are plotted as averages.

FIG. 6. Cellular cytotoxicity of IB:10:D (1) and 2-273 in MCF-7 and T47D. (A) MCF-7 cells were incubated with compound for 72 hours and viability measured via alamar blue fluorescence. Raptinal (100 μM) was used as the 100% dead control. Data is shown as mean±s.e.m.; n=3 independent replicates. (B) T47D cells were incubated with compound for 72 hours and viability measured via alamar blue fluorescence. Raptinal (100 μM) was used as the 100% dead control. Data is shown as mean±s.e.m.; n=3 independent replicates. (C) Cellular cytotoxicity of compound 34 when incubated with MCF-7 (left panel) and T47D (right panel).

FIG. 7. Whole cell CS levels are modulated by treatment with 2-273. (A) Whole cell CS levels are decreased after treatment with either IB:10:D (top) and 2-273 (bottom) compared to cells treated with DMSO in MCF-7. Cells were incubated with 6.25 μM compound for 72 hours. 1.5×10⁶ cells were lysed and submitted to LC-MS/MS analysis for CS quantification. Data plotted as mean±s.e.m.; n=3 independent replicates. Percent decrease was calculated as: ((average [CS_{DMSO}]−average [CS_{treated}])/average [CS_{DMSO}])×100. (B) CS levels in T47D are decreased after incubation with 6.25 μM 2-273 for 72 hours. Cells were collected as described in A. Data plotted as mean±s.e.m.; n=3 independent replicates. (C) Whole cell CS levels in MCF-7 after treatment with 6.25 μM galeterone for 72 hours. Cells were collected as described in A. Data plotted as mean±s.e.m.; n=3 independent replicates. (D) Whole cell CS levels in MCF-7 are not changed after treatment with 3-67, a compound deemed inactive from in vitro data, contrary to treatment with 2-273. Cells were treated with DMSO or 6.25 μM 3-67 or 2-273 for 72 hours. Cells were collected as described in A. Data plotted as mean±s.e.m.; n=3 independent replicates. (E) Whole cell CS levels in MCF-7 are not decreased upon treatment with the apoptosis inducing compound, Raptinal. Cells were treated at 0.156 μM Raptinal for 72 hours, and collected as described in A. Data plotted as mean±s.e.m.; n=3 independent replicates. Statistics were determined with a two-tailed student t-test; ns: not significant, *p<0.05, **p<0.01, ***p<0.001, ****p<0.0001.

FIG. 8. 2-273 treatment inhibits CS production but not decreases in overall cholesterol level nor result in expression level changes for key CS modulating enzymes. (A) Free cholesterol levels in MCF-7 do not show a significant decrease after treatment with 6.25 μM either 2-273 or 3-67 as compared to DMSO. Cholesterol levels were measured after 72 hours of incubation with compound. Data plotted as mean±s.e.m.; n=3 independent replicates. Note: No changes in expression levels of STS or SULT2B1b are observed in MCF-7 or T47D after treatment with 6.25 μM 2-273 for 72 hours. (B) CS level dose response in MCF-7 after 72 hour treatment with 2-273. 1.5×10⁶ cells were lysed and submitted to LC-MS/MS analysis for CS quantification. Data plotted as mean±s.e.m.; n=3 independent replicates. (C) Decreased whole cell CS levels in MCF-7 after varying treatment times with 6.25 μM 2-273. Cells were collected as described in C. Data plotted as mean±s.e.m.; n=3 independent replicates. (D) Decreased CS levels are observed in MCF-7 after 24 hour treatment with 6.25 μM 2-273, following removal of compound whole cell CS levels remain depleted up to 48 hours later. Cells were collected as described in C. Data plotted as mean±s.e.m.; n=3 independent replicates. Statistics were determined with a two-tailed student t-test; ns, not significant.

FIG. 9. The cell line MDG is suitable for in vivo model systems. (A) Whole cell CS levels in MYS or MDG cell lines. 1.5×10⁶ cells were lysed and submitted to LC-MS/MS analysis for CS quantification. Data plotted as mean±s.e.m.; n=3 independent replicates. (B) Decreased whole cell CS levels after treatment with 6.25 μM 2-273 for 72 hours is recapitulated in MDG. Cells were collected as described in A. Data plotted as mean±s.e.m.; n=3 biological replicates. Statistics were determined with a two-tailed student t-test; ns: not significant, *p<0.05.

FIG. 10. Results from pharmacokinetic experiment in C57B1/6 mice treated with compound 2 at 200 mg/kg IP. Data plotted as mean±s.e.m.; n=3 mice per time point.

FIG. 11. CS levels are higher in smaller MDG tumors. CS levels in tumors were analyzed across tumor sizes. MDG cells (1.5×10⁶ or 3×10⁶) were injected into the mammary fat pad of athymic nude mice. Tumors were established and grew for 39 days. Tumors were harvested, flash frozen, and homogenized in ice cold methanol. The soluble portion was analyzed by LC-MS/MS to yield the concentration of CS in the samples which was normalized based on mass of tumor tissue. Data plotted as mean±s.d.; n=3.

### DETAILED DESCRIPTION

CS levels are modulated by two complementary enzymes, cholesterol sulfotransferase (SULT2B1b), which catalyzes the reaction between 3′-phosphoadenosine-5′-phosphosulfate (PAPS) and cholesterol to synthesize CS and forming 3′-phosphoadenosine-5′-phosphate (PAP) as a byproduct, and steroid sulfatase (STS) which catalyzes the removal of the sulfate moiety. SULT2B1 (gene encoding SULT2B1b) knockout leads to no production of CS (Chart 1). It should be noted that a second gene product, SULT2B1a, formed from alternative splicing of the first exon, has been reported. While this isoform shows a mild ability to sulfonate cholesterol, endogenous expression of SULT2B1a has not been demonstrated at the protein level. This suggests SULT2B1b is the sole enzyme responsible for CS production in the cell. While much work has been done on clinical development of STS inhibitors for the treatment of estrogen- and androgen-dependent cancers, small molecule inhibition of SULT2B1b has remained unexplored even though it is widely reported to be overexpressed in a variety of cancers, e.g., colorectal carcinoma, hepatocellular carcinoma, gastric cancer, endometrial cancer, and breast cancer.

Based on the striking overexpression of SULT2B1b observed across multiple cancer types and its exclusive role in producing CS, a hypothesis was formulated that SULT2B1b overexpression and CS production thereof may be another mechanism cancers employ to evade and escape the immune system. Therefore, modulation of CS levels within the tumor microenvironment could be key to restoring immune system function. However, there is a dearth of chemical tools to explore this therapeutic approach. Galeterone is the only molecule reported to date which is known to inhibit SULT2B1b and other sulfotransferases, in addition to its previously recognized activity against the androgen receptor amid other biological effects.

Chart 1. SULT2B1b catalyzes the formation of cholesterol sulfate. The reverse reaction, catalyzed by steroid sulfatase (STS), results in the re-formation of cholesterol. PAPS=3′-phosphoadenosine-5′-phosphosulfate; PAP=3′-phosphoadenosine-5′-phosphate.

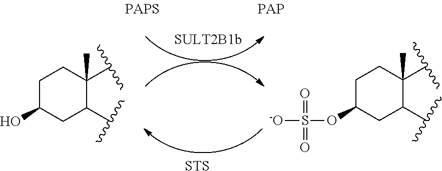

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R. J. Lewis, John Wiley & Sons, New York, N.Y., 2001.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit. For example, one or more substituents on a phenyl ring refers to one to five, or one to four, for example if the phenyl ring is disubstituted.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements. When values are expressed as approximations, by use of the antecedent “about,” it will be understood that the particular value without the modifier “about” also forms a further aspect.

The terms “about” and “approximately” are used interchangeably. Both terms can refer to a variation of ±5%, ±10%, ±20%, or ±25% of the value specified. For example, “about 50” percent can in some embodiments carry a variation from 45 to 55 percent, or as otherwise defined by a particular claim. For integer ranges, the term “about” can include one or two integers greater than and/or less than a recited integer at each end of the range. Unless indicated otherwise herein, the terms “about” and “approximately” are intended to include values, e.g., weight percentages, proximate to the recited range that are equivalent in terms of the functionality of the individual ingredient, composition, or embodiment. The terms “about” and “approximately” can also modify the end-points of a recited range as discussed above in this paragraph.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. It is therefore understood that each unit between two particular units are also disclosed. For example, if 10 to 15 is disclosed, then 11, 12, 13, and 14 are also disclosed, individually, and as part of a range. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents. It will be further understood that the endpoints of each of the ranges are significant both in relation to the other endpoint, and independently of the other endpoint.

This disclosure provides ranges, limits, and deviations to variables such as volume, mass, percentages, ratios, etc. It is understood by an ordinary person skilled in the art that a range, such as “number1” to “number2”, implies a continuous range of numbers that includes the whole numbers and fractional numbers. For example, 1 to 10 means 1, 2, 3, 4, 5, . . . 9, 10. It also means 1.0, 1.1, 1.2, 1.3, . . . , 9.8, 9.9, 10.0, and also means 1.01, 1.02, 1.03, and so on. If the variable disclosed is a number less than “number10”, it implies a continuous range that includes whole numbers and fractional numbers less than number10, as discussed above. Similarly, if the variable disclosed is a number greater than “number10”, it implies a continuous range that includes whole numbers and fractional numbers greater than number10. These ranges can be modified by the term “about”, whose meaning has been described above.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

An “effective amount” refers to an amount effective to treat a disease, disorder, and/or condition, or to bring about a recited effect. For example, an effective amount can be an amount effective to reduce the progression or severity of the condition or symptoms being treated. Determination of a therapeutically effective amount is well within the capacity of persons skilled in the art. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect.

Alternatively, The terms “effective amount” or “therapeutically effective amount,” as used herein, refer to a sufficient amount of an agent or a composition or combination of compositions being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study. The dose could be administered in one or more administrations. However, the precise determination of what would be considered an effective dose may be based on factors individual to each patient, including, but not limited to, the patient's age, size, type or extent of disease, stage of the disease, route of administration of the compositions, the type or extent of supplemental therapy used, ongoing disease process and type of treatment desired (e.g., aggressive vs. conventional treatment).

The terms “treating”, “treat” and “treatment” include (i) preventing a disease, pathologic or medical condition from occurring (e.g., prophylaxis); (ii) inhibiting the disease, pathologic or medical condition or arresting its development; (iii) relieving the disease, pathologic or medical condition; and/or (iv) diminishing symptoms associated with the disease, pathologic or medical condition. Thus, the terms “treat”, “treatment”, and “treating” can extend to prophylaxis and can include prevent, prevention, preventing, lowering, stopping or reversing the progression or severity of the condition or symptoms being treated. As such, the term “treatment” can include medical, therapeutic, and/or prophylactic administration, as appropriate.

As used herein, “subject” or “patient” means an individual having symptoms of, or at risk for, a disease or other malignancy. A patient may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods provided herein, the mammal is a human.

As used herein, the terms “providing”, “administering,” “introducing,” are used interchangeably herein and refer to the placement of a compound of the disclosure into a subject by a method or route that results in at least partial localization of the compound to a desired site. The compound can be administered by any appropriate route that results in delivery to a desired location in the subject.

The compound and compositions described herein may be administered with additional compositions to prolong stability and activity of the compositions, or in combination with other therapeutic drugs.

The terms “inhibit”, “inhibiting”, and “inhibition” refer to the slowing, halting, or reversing the growth or progression of a disease, infection, condition, or group of cells. The inhibition can be greater than about 20%, 40%, 60%, 80%, 90%, 95%, or 99%, for example, compared to the growth or progression that occurs in the absence of the treatment or contacting.

The term “substantially” as used herein, is a broad term and is used in its ordinary sense, including, without limitation, being largely but not necessarily wholly that which is specified. For example, the term could refer to a numerical value that may not be 100% the full numerical value. The full numerical value may be less by about 1%, about 2%, about 3%, about 4%, about 5%, about 6%, about 7%, about 8%, about 9%, about 10%, about 15%, or about 20%.

Wherever the term “comprising” is used herein, options are contemplated wherein the terms “consisting of” or “consisting essentially of” are used instead. As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the aspect element. As used herein, “consisting essentially of” does not exclude materials or steps that do not materially affect the basic and novel characteristics of the aspect. In each instance herein any of the terms “comprising”, “consisting essentially of” and “consisting of” may be replaced with either of the other two terms. The disclosure illustratively described herein may be suitably practiced in the absence of any element or elements, limitation or limitations which is not specifically disclosed herein.

This disclosure provides methods of making the compounds and compositions of the invention. The compounds and compositions can be prepared by any of the applicable techniques described herein, optionally in combination with standard techniques of organic synthesis. Many techniques such as etherification and esterification are well known in the art. However, many of these techniques are elaborated in Compendium of Organic Synthetic Methods (John Wiley & Sons, New York), Vol. 1, Ian T. Harrison and Shuyen Harrison, 1971; Vol. 2, Ian T. Harrison and Shuyen Harrison, 1974; Vol. 3, Louis S. Hegedus and Leroy Wade, 1977; Vol. 4, Leroy G. Wade, Jr., 1980; Vol. 5, Leroy G. Wade, Jr., 1984; and Vol. 6; as well as standard organic reference texts such as March's Advanced Organic Chemistry: Reactions, Mechanisms, and Structure, 5th Ed., by M. B. Smith and J. March (John Wiley & Sons, New York, 2001); Comprehensive Organic Synthesis. Selectivity, Strategy & Efficiency in Modern Organic Chemistry. In 9 Volumes, Barry M. Trost, Editor-in-Chief (Pergamon Press, New York, 1993 printing); Advanced Organic Chemistry, Part B: Reactions and Synthesis, Second Edition, Cary and Sundberg (1983); for heterocyclic synthesis see Hermanson, Greg T., Bioconjugate Techniques, Third Edition, Academic Press, 2013.

The formulas and compounds described herein can be modified using protecting groups. Suitable amino and carboxy protecting groups are known to those skilled in the art (see for example, Protecting Groups in Organic Synthesis, Second Edition, Greene, T. W., and Wutz, P. G. M., John Wiley & Sons, New York, and references cited therein; Philip J. Kocienski; Protecting Groups (Georg Thieme Verlag Stuttgart, New York, 1994), and references cited therein); and Comprehensive Organic Transformations, Larock, R. C., Second Edition, John Wiley & Sons, New York (1999), and referenced cited therein.

The term “halo” or “halide” refers to fluoro, chloro, bromo, or iodo. Similarly, the term “halogen” refers to fluorine, chlorine, bromine, and iodine.

The term “alkyl” refers to a branched or unbranched hydrocarbon having, for example, from 1-20 carbon atoms, and often 1-12, 1-10, 1-8, 1-6, or 1-4 carbon atoms; or for example, a range between 1-20 carbon atoms, such as 2-6, 3-6, 2-8, or 3-8 carbon atoms. As used herein, the term “alkyl” also encompasses a “cycloalkyl”, defined below. Examples include, but are not limited to, methyl, ethyl, 1-propyl, 2-propyl (iso-propyl), 1-butyl, 2-methyl-1-propyl (isobutyl), 2-butyl (sec-butyl), 2-methyl-2-propyl (t-butyl), 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, hexyl, octyl, decyl, dodecyl, and the like. The alkyl can be unsubstituted or substituted, for example, with a substituent described below or otherwise described herein. The alkyl can also be optionally partially or fully unsaturated. As such, the recitation of an alkyl group can include an alkenyl group or an alkynyl group. The alkyl can be a monovalent hydrocarbon radical, as described and exemplified above, or it can be a divalent hydrocarbon radical (i.e., an alkylene).

An alkylene is an alkyl group having two free valences at a carbon atom or two different carbon atoms of a carbon chain. Similarly, alkenylene and alkynylene are respectively an alkene and an alkyne having two free valences at two different carbon atoms.

The term “cycloalkyl” refers to cyclic alkyl groups of, for example, from 3 to 10 carbon atoms having a single cyclic ring or multiple condensed rings. Cycloalkyl groups include, by way of example, single ring structures such as cyclopropyl, cyclobutyl, cyclopentyl, cyclooctyl, and the like, or multiple ring structures such as adamantyl, and the like. The cycloalkyl can be unsubstituted or substituted. The cycloalkyl group can be monovalent or divalent, and can be optionally substituted as described for alkyl groups. The cycloalkyl group can optionally include one or more cites of unsaturation, for example, the cycloalkyl group can include one or more carbon-carbon double bonds, such as, for example, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, and the like.

The term “heteroatom” refers to any atom in the periodic table that is not carbon or hydrogen. Typically, a heteroatom is O, S, N, P. The heteroatom may also be a halogen, metal or metalloid.

The term “heterocycloalkyl” or “heterocyclyl” refers to a saturated or partially saturated monocyclic, bicyclic, or polycyclic ring containing at least one heteroatom selected from nitrogen, sulfur, oxygen, preferably from 1 to 3 heteroatoms in at least one ring. Each ring is preferably from 3 to 10 membered, more preferably 4 to 7 membered. Examples of suitable heterocycloalkyl substituents include pyrrolidyl, tetrahydrofuryl, tetrahydrothiofuranyl, piperidyl, piperazyl, tetrahydropyranyl, morpholino, 1,3-diazapane, 1,4-diazapane, 1,4-oxazepane, and 1,4-oxathiapane. The group may be a terminal group or a bridging group.

The term “aryl” refers to an aromatic hydrocarbon group derived from the removal of at least one hydrogen atom from a single carbon atom of a parent aromatic ring system. The radical attachment site can be at a saturated or unsaturated carbon atom of the parent ring system. The aryl group can have from 6 to 30 carbon atoms, for example, about 6-10 carbon atoms. The aryl group can have a single ring (e.g., phenyl) or multiple condensed (fused) rings, wherein at least one ring is aromatic (e.g., naphthyl, dihydrophenanthrenyl, fluorenyl, or anthryl). Typical aryl groups include, but are not limited to, radicals derived from benzene, naphthalene, anthracene, biphenyl, and the like. The aryl can be unsubstituted or optionally substituted with a substituent described below.

The term “heteroaryl” refers to a monocyclic, bicyclic, or tricyclic ring system containing one, two, or three aromatic rings and containing at least one nitrogen, oxygen, or sulfur atom in an aromatic ring. The heteroaryl can be unsubstituted or substituted, for example, with one or more, and in particular one to three, substituents, as described in the definition of “substituted”. Typical heteroaryl groups contain 2-20 carbon atoms in the ring skeleton in addition to the one or more heteroatoms, wherein the ring skeleton comprises a 5-membered ring, a 6-membered ring, two 5-membered rings, two 6-membered rings, or a 5-membered ring fused to a 6-membered ring. Examples of heteroaryl groups include, but are not limited to, 2H-pyrrolyl, 3H-indolyl, 4H-quinolizinyl, acridinyl, benzo[b]thienyl, benzothiazolyl, β-carbolinyl, carbazolyl, chromenyl, cinnolinyl, dibenzo[b,d]furanyl, furazanyl, furyl, imidazolyl, imidizolyl, indazolyl, indolisinyl, indolyl, isobenzofuranyl, isoindolyl, isoquinolyl, isothiazolyl, isoxazolyl, naphthyridinyl, oxazolyl, perimidinyl, phenanthridinyl, phenanthrolinyl, phenarsazinyl, phenazinyl, phenothiazinyl, phenoxathiinyl, phenoxazinyl, phthalazinyl, pteridinyl, purinyl, pyranyl, pyrazinyl, pyrazolyl, pyridazinyl, pyridyl, pyrimidinyl, pyrrolyl, quinazolinyl, quinolyl, quinoxalinyl, thiadiazolyl, thianthrenyl, thiazolyl, thienyl, triazolyl, tetrazolyl, and xanthenyl. In one embodiment the term “heteroaryl” denotes a monocyclic aromatic ring containing five or six ring atoms containing carbon and 1, 2, 3, or 4 heteroatoms independently selected from non-peroxide oxygen, sulfur, and N(Z) wherein Z is absent or is H, O, alkyl, aryl, or (C₁-C₆)alkylaryl. In some embodiments, heteroaryl denotes an ortho-fused bicyclic heterocycle of about eight to ten ring atoms derived therefrom, particularly a benz-derivative or one derived by fusing a propylene, trimethylene, or tetramethylene diradical thereto.

As used herein, the term “substituted” or “substituent” is intended to indicate that one or more (for example, in various embodiments, 1-10; in other embodiments, 1-6; in some embodiments 1, 2, 3, 4, or 5; in certain embodiments, 1, 2, or 3; and in other embodiments, 1 or 2) hydrogens on the group indicated in the expression using “substituted” (or “substituent”) is replaced with a selection from the indicated group(s), or with a suitable group known to those of skill in the art, provided that the indicated atom's normal valency is not exceeded, and that the substitution results in a stable compound. Suitable indicated groups include, e.g., alkyl, alkenyl, alkynyl, alkoxy, haloalkyl, hydroxyalkyl, aryl, heteroaryl, heterocyclyl, cycloalkyl, alkanoyl, alkoxycarbonyl, amino, alkylamino, dialkylamino, carboxyalkyl, alkylthio, alkylsulfinyl, and alkylsulfonyl. Substituents of the indicated groups can be those recited in a specific list of substituents described herein, or as one of skill in the art would recognize, can be one or more substituents selected from alkyl, alkenyl, alkynyl, alkoxy, halo, haloalkyl, hydroxy, hydroxyalkyl, aryl, heteroaryl, heterocycle, cycloalkyl, alkanoyl, alkoxycarbonyl, amino, alkylamino, dialkylamino, trifluoromethylthio, difluoromethyl, acylamino, nitro, trifluoromethyl, trifluoromethoxy, carboxy, carboxyalkyl, keto, thioxo, alkylthio, alkylsulfinyl, alkylsulfonyl, and cyano. Suitable substituents of indicated groups can be bonded to a substituted carbon atom include F, Cl, Br, I, OR′, OC(O)N(R′)2, CN, CF3, OCF3, R′, O, S, C(O), S(O), methylenedioxy, ethylenedioxy, N(R′)2, SR′, SOR′, SO2R′, SO2N(R′)2, SO3R′, C(O)R′, C(O)C(O)R′, C(O)CH2C(O)R′, C(S)R′, C(O)OR′, OC(O)R′, C(O)N(R′)2, OC(O)N(R′)2, C(S)N(R′)2, (CH2)0-2NHC(O)R′, N(R′)N(R′)C(O)R′, N(R′)N(R′)C(O)OR′, N(R′)N(R′)CON(R′)2, N(R′)SO2R′, N(R′)SO2N(R′)2, N(R′)C(O)OR′, N(R′)C(O)R′, N(R′)C(S)R′, N(R′)C(O)N(R′)2, N(R′)C(S)N(R′)2, N(COR′)COR′, N(OR′)R′, C(═NH)N(R′)2, C(O)N(OR′)R′, or C(═NOR′)R′ wherein R′ can be hydrogen or a carbon-based moiety (e.g., (C1-C6)alkyl), and wherein the carbon-based moiety can itself be further substituted. When a substituent is monovalent, such as, for example, F or Cl, it is bonded to the atom it is substituting by a single bond. When a substituent is divalent, such as 0, it is bonded to the atom it is substituting by a double bond; for example, a carbon atom substituted with O forms a carbonyl group, C═O.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds of the invention may contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof, such as racemic mixtures, which form part of the present invention. Many organic compounds exist in optically active forms, i.e., they have the ability to rotate the plane of plane-polarized light. In describing an optically active compound, the prefixes D and L, or R and S. are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate (defined below), which may occur where there has been no stereoselection or stereospecificity in a chemical reaction or process.

The terms “racemic mixture” and “racemate” refer to an equimolar mixture of two enantiomeric species, devoid of optical activity.

The term “enantiomerically enriched” (“ee”) as used herein refers to mixtures that have one enantiomer present to a greater extent than another. Reactions that provide one enantiomer present to a greater extent than another would therefore be “enantioselective” (or demonstrate “enantioselectivity”). In one embodiment of the invention, the term “enantiomerically enriched” refers to a mixture having at least about 2% ee; in another embodiment of the invention, the term “enantiomerically enriched” refers to a mixture having at least about 5% ee; in another embodiment of the invention, the term “enantiomerically enriched” refers to a mixture having at least about 20%; in another embodiment of the invention, the term “enantiomerically enriched” refers to a mixture having at least about 50%; in another embodiment of the invention, the term “enantiomerically enriched” refers to a mixture having at least about 80%; in another embodiment of the invention, the term “enantiomerically enriched” refers to a mixture having at least about 90%; in another embodiment of the invention, the term “enantiomerically enriched” refers to a mixture having at least about 95%; in another embodiment of the invention, the term “enantiomerically enriched” refers to a mixture having at least about 98%; in another embodiment of the invention, the term “enantiomerically enriched” refers to a mixture having at least about 99%. The term “enantiomerically enriched” includes enantiomerically pure mixtures which are mixtures that are substantially free of the species of the opposite optical activity or one enantiomer is present in very low quantities, for example, 0.01%, 0.001% or 0.0001%.

The term “IC₅₀” is generally defined as the concentration required to kill 50% of the cells in 24 hours.

Certain compounds herein have more than one compound number identifier: compound 1 is IB:10:D; compound 3 is 2-273; and compound 6 is 3-67.

### Embodiments of the Technology

This disclosure provides a compound of Formula I:

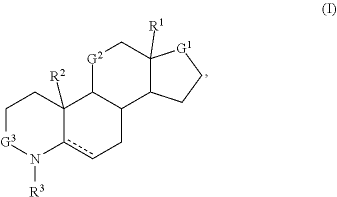

or a salt thereof,
wherein
[symbol] is an unsaturated or saturated bond;
G¹ is C═O, CHF, CF₂, CHJ¹R^{a}, C(OCH₂)₂;
   J¹ is O, S, NR^{c}, C(O)NR^{d};
G² is C═O, CH₂, CHF, CF₂, CHJ²R^{b}, or C(OCH₂)₂;
   J² is O, S, NR^{g}, C(O)X² wherein X² is O or NR^{h};
G³ is C═O or CH₂;
R^{a}, R^{b}, R^{c}, R^{d}, R^{g}, and R^{h} are each independently H, or -(C₁-C₆)alkyl;
R¹ is -(C₁-C₆)alkyl;
R² is -(C₁-C₆)alkyl;
R³ is —CH₂R⁴, —CH(CH₃)R⁴, R⁴, or —C(O)R⁴ wherein R³ is not —C(O)R⁴ when G³ is C═O; and
R⁴ is aryl, heteroaryl, cycloalkyl, or heterocyclyl, wherein aryl and heteroaryl are each optionally substituted with one or more substituents.

In some embodiments, the compound of Formula I is not (4aR,4bS,6aS,9aS,9bS)-1-(4-chlorobenzyl)-4a,6a-dimethyl-3,4,4a,6,6a,8,9,9a,9b,10-decahydro-1H-indeno[5,4-f]quinoline-2,5,7(4bH)-trione or (4aR,4bS,6aS,9aS,9bS)-1-benzyl-4a,6a-dimethyl-3,4,4a,6,6a,8,9,9a,9b,10-decahydro-1H-indeno[5,4-f]quinoline-2,5,7(4bH)-trione.

In further embodiments, G¹ is CN when the bond between G¹ and the carbon atom substituted with R¹ is absent and the carbon atom substituted with R¹ is unsaturated. In some embodiments, said carbon atom is unsaturated wherein a double bond exists between the carbon atom and another carbon atom that is alpha to G².

In various embodiments, R⁴ is:

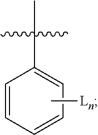

wherein
each Lₙ is independently halo, X³R^{e} or -(C₁-C₆)alkyl;
n is 1, 2, 3, 4, 5, or 0;
each X³ is independently O, S, or NR^{f}; and
   R^{e} and R^{f} are each independently H or -(C₁-C₆)alkyl.

In various embodiments, one L is in the para-position. In various embodiments, G¹ is C═O or CHOH. In various embodiments, G² and G³ are C═O. In various embodiments, R¹ and R² are CH₃.

In various embodiments, R³ is:

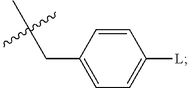

wherein
L is halo, X³R^{e}, or -(C₁-C₆)alkyl;
X³ is O, S, or NR^{f}; and
   R^{e} and R^{f} are H or -(C₁-C₆)alkyl.

In some embodiments, the compound of Formula I is represented by Formula II:

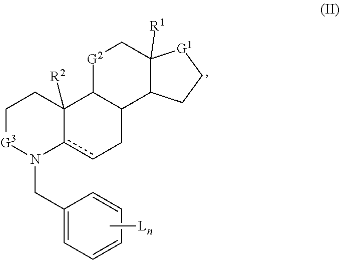

or a pharmaceutically acceptable salt thereof;
wherein
G¹ is C═O or CHJ¹R^{a};
G² is C═O or CHJ²R^{b};
each Lₙ is independently halo, X³R^{e} or -(C₁-C₆)alkyl;
n is 1 or 2;
each X³ is independently O, S, or NR^{f}; and
R^{e} and R^{f} are each independently H or -(C₁-C₆)alkyl;
   wherein each -(C₁-C₆)alkyl is optionally substituted with one or more substituents.

In some embodiments, the compound of Formula I is represented by Formula III:

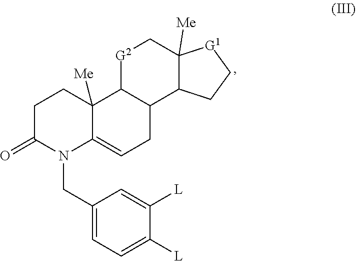

or a pharmaceutically acceptable salt thereof;
wherein
G¹ is C═O or CHJ¹R^{a};
G² is C═O or CHJ²R^{b};
each L is independently halo, X³R^{e}, -(C₁-C₆)alkyl, or H;
each X³ is independently O, S, or NR^{f}; and
   R^{e} and R^{f} are each independently H or -(C₁-C₆)alkyl.

In some embodiments, the compound of Formula I is represented by Formula IV:

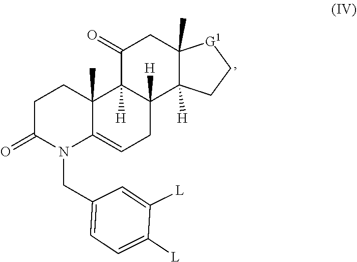

or a pharmaceutically acceptable salt thereof;
wherein
each L is independently halo, X³R^{e}, -(C₁-C₆)alkyl, or H;
each X³ is independently O, S, or NR^{f}; and
   R^{e} and R^{f} are each independently H or -(C₁-C₆)alkyl.

In some embodiments, the compound of Formula I is represented by Formula V:

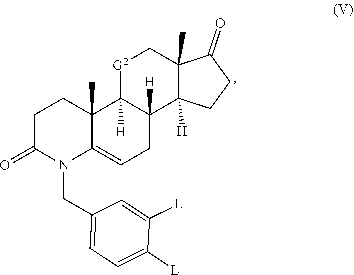

or a pharmaceutically acceptable salt thereof;
wherein
each L is independently halo, X³R^{e}, -(C₁-C₆)alkyl, or H;
each X³ is independently O, S, or NR^{f}; and
   R^{e} and R^{f} are each independently H or -(C₁-C₆)alkyl.

In some embodiments, the compound is (3):

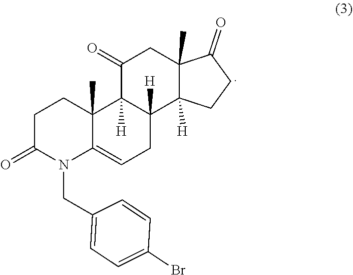

In some embodiments, the compound is (34):

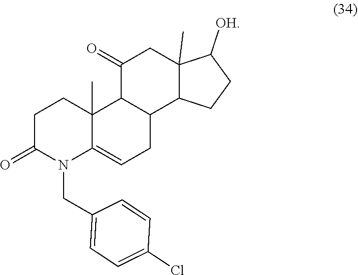 [SMILES: CC12CCC(=O)N(Cc3ccc(Cl)cc3)C1=CCC1C2C(=O)CC2(C)C(O)CCC12]

Also, this disclosure provides a composition comprising a compound disclosed herein and a pharmaceutically acceptable excipient.

This disclosure also provides a method for in-vivo inhibition of an enzyme comprising contacting a compound or composition disclosed herein and the enzyme sulfotransferase family cytosolic 2B member 1 (SULT2B1b) wherein the in-vivo inhibition of the enzyme suppresses production of cholesterol sulfate.

Additionally, this disclosure provides a method for inducing death of cancer cells comprising contacting a compound or composition disclosed herein and the cancer cells wherein the compound initiates an immune response by lowering levels of cholesterol sulfate via inhibition of the enzyme SULT2B1b, thereby inducing an immune response mediated death of the cancer cells. In various embodiments, the cancer cells overexpress SULT2B1b. In various embodiments, breast cancer cells, endometrial cancer cells, liver cancer cells, colorectal cancer cells, or gastrointestinal cancer cells.

Furthermore, this disclosure provides a method for treatment of cancer comprising, administering to a subject in need of cancer treatment a compound of Formula I.

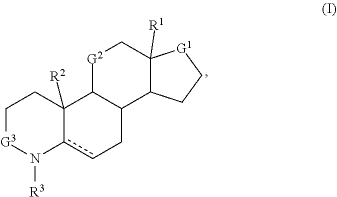

or a pharmaceutically acceptable salt thereof;
wherein
[symbol] is an unsaturated or saturated bond;
G¹ is C═O, CH₂, CHF, CF₂, CHJ¹R^{a}, or C(OCH₂)₂;
   J¹ is O, S, NR^{c}, C(O)X¹ wherein X¹ is O or NR^{d};
G² is C═O, CH₂, CHF, CF₂, CHJ²R^{b}, or C(OCH₂)₂;
   J² is O, S, NR^{g}, C(O)X² wherein X² is O or NR^{h};
G³ is C═O or CH₂;
R^{a}, R^{b}, R, R^{d}, R^{g}, and R^{h} are each independently H, or -(C₁-C₆)alkyl;
R¹ is -(C₁-C₆)alkyl;
R² is -(C₁-C₆)alkyl;
R³ is —CH₂R⁴, —CH(CH₃)R⁴, R⁴, —C(O)R⁴, or H wherein R³ is not —C(O)R⁴ when G³ is C═O; and
R⁴ is aryl, heteroaryl, cycloalkyl, heterocyclyl, or -(C₁-C₆)alkyl, wherein aryl, heteroaryl, cycloalkyl, and heterocyclyl are each optionally substituted with one or more substituents;
wherein each -(C₁-C₆)alkyl is independently saturated or unsaturated, and optionally substituted with one or more substituents;
wherein the compound increases T-cell activity via inhibition of the enzyme SULT2B1b, wherein immune clearance of the cancer increases in the subject, thereby treating the cancer.

In other embodiments, R⁴ is:

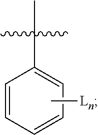

wherein
each Lₙ is independently halo, X³R^{e} or -(C₁-C₆)alkyl;
n is 1, 2, 3, 4, 5, or 0;
each X³ is independently O, S, or NR^{f}; and
   R^{e} and R^{f} are each independently H or -(C₁-C₆)alkyl.

In some embodiments, the compound and a second agent are simultaneously or sequentially administered to the subject for the treatment of the cancer. In some embodiments, a combination of the compound and the second agent have synergistic anti-cancer activity. In various embodiments, the cancer overexpresses SULT2B1b. In some embodiments, the cancer is breast cancer, endometrial cancer, liver cancer, colorectal cancer, or gastrointestinal cancer.

In some embodiments, the compound is (4aR,4bS,6aS,9aS,9bS)-1-(4-bromobenzyl)-4a,6a-dimethyl-3,4,4a,6,6a,8,9,9a,9b,10-decahydro-1H-indeno[5,4-f]quinoline-2,5,7(4bH)-trione (3); or (4aR,4bS,6aS,7S,9aS,9bS)-1-(4-bromobenzyl)-7-hydroxy-4a,6a-dimethyl-3,4,4a,4b,6,6a,7,8,9,9a,9b,10-dodecahydro-1H-indeno[5,4-f]quinoline-2,5-dione (34). In some other embodiments, the compound is 3-((6aS,7S,10aS,10bR)-4-(4-bromobenzyl)-8,10b-dimethyl-3,10-dioxo-1,2,3,4,6,6a,7,10,10a,10b-decahydrobenzo[f]quinolin-7-yl)propanenitrile (36). In various other embodiments, the compound is any one of the compounds in Chart 2.

Chart 2. Exemplary compounds embodied by the technology.

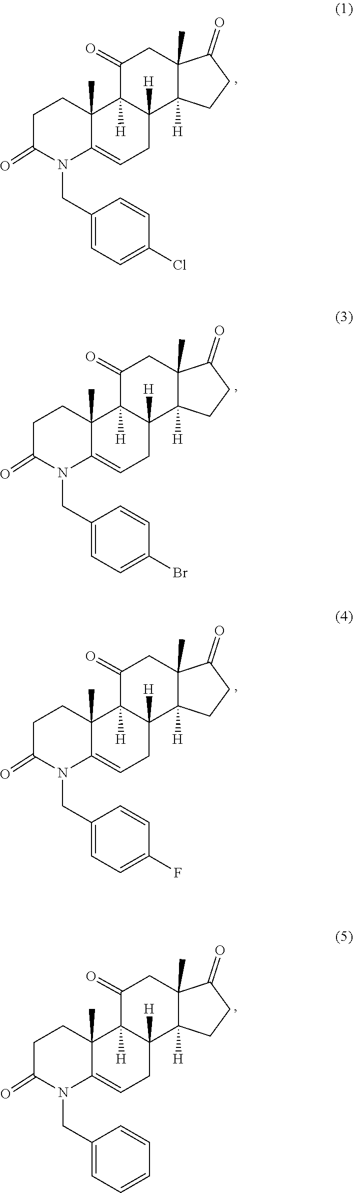

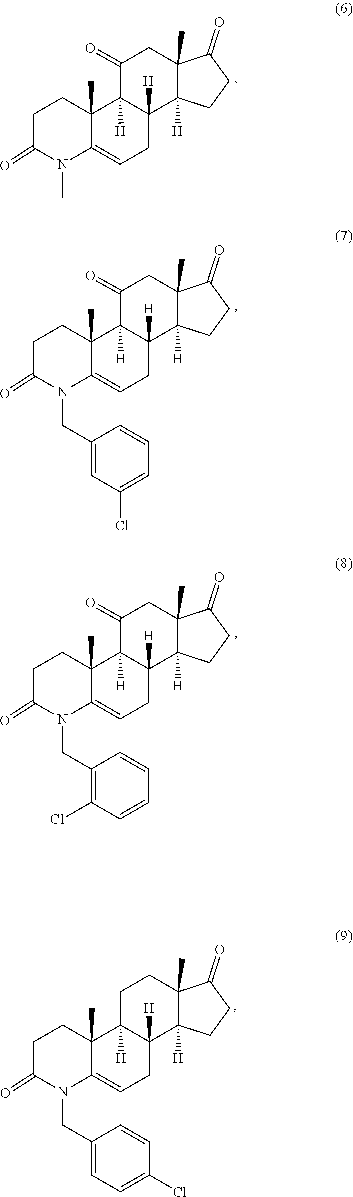

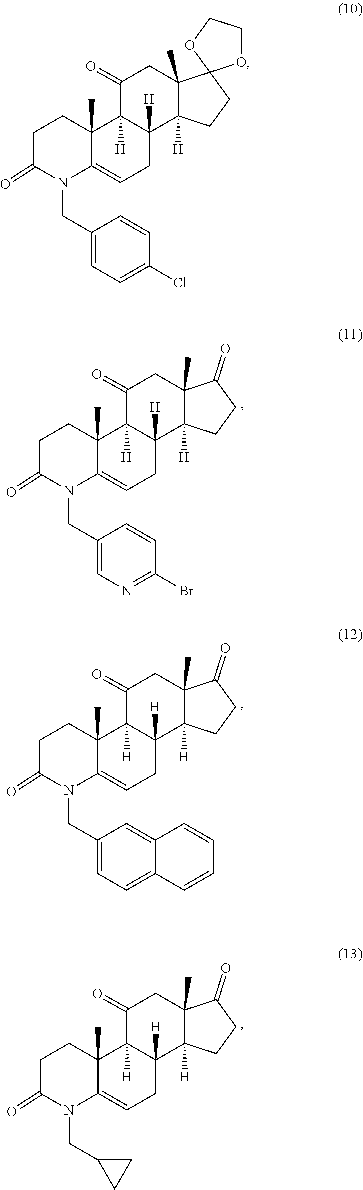

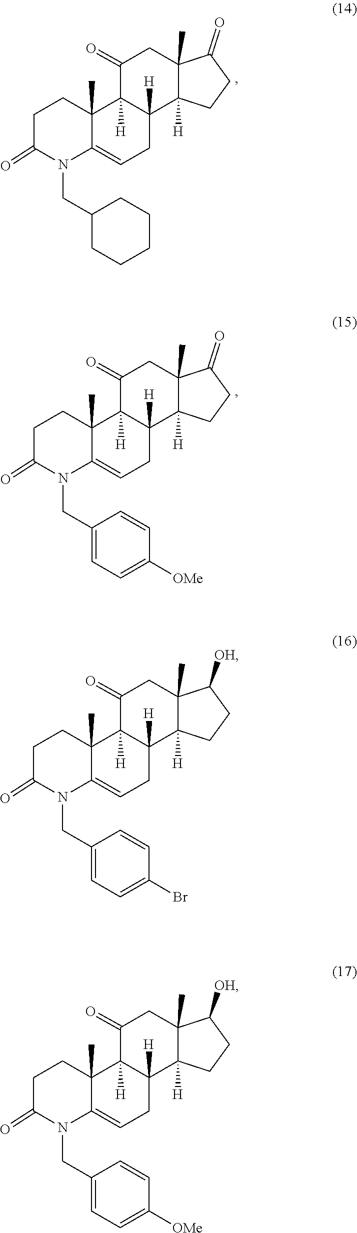

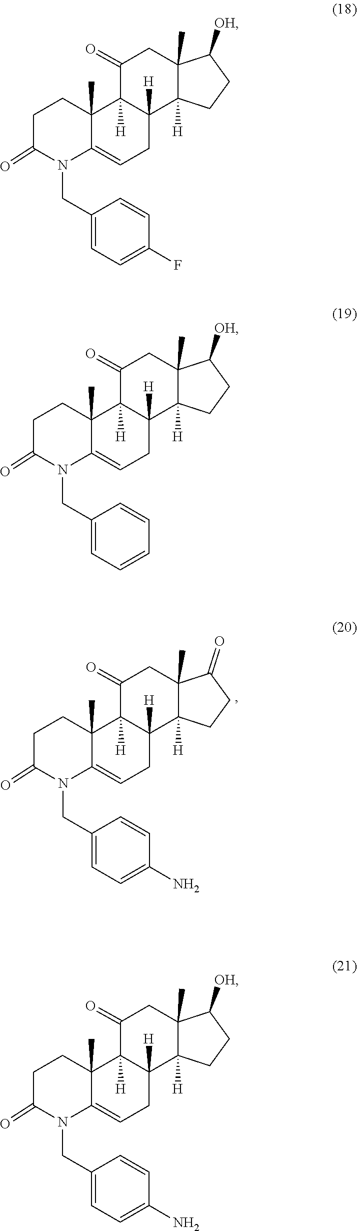

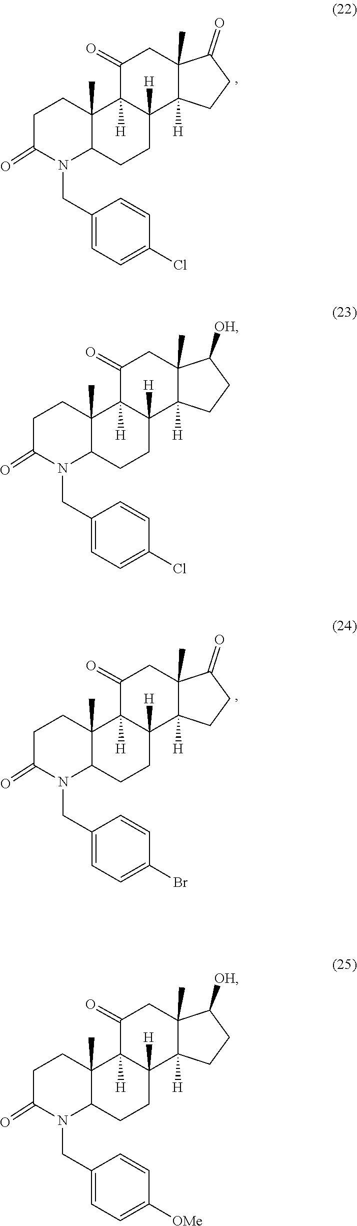

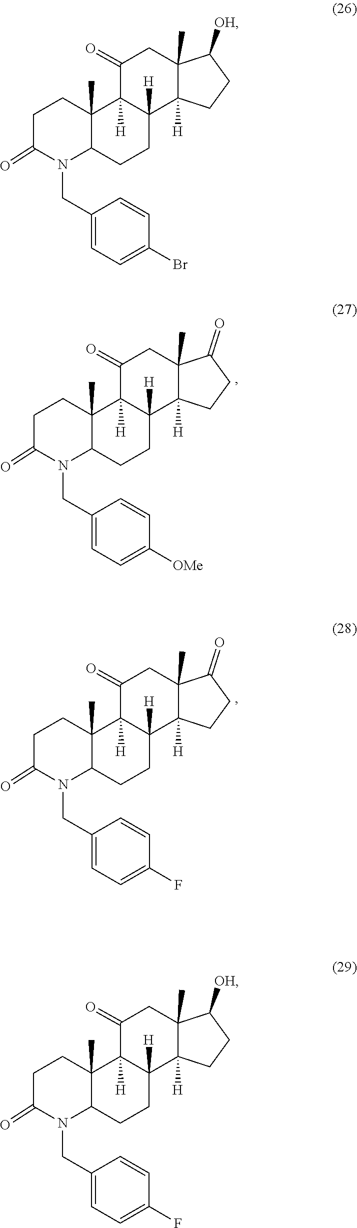

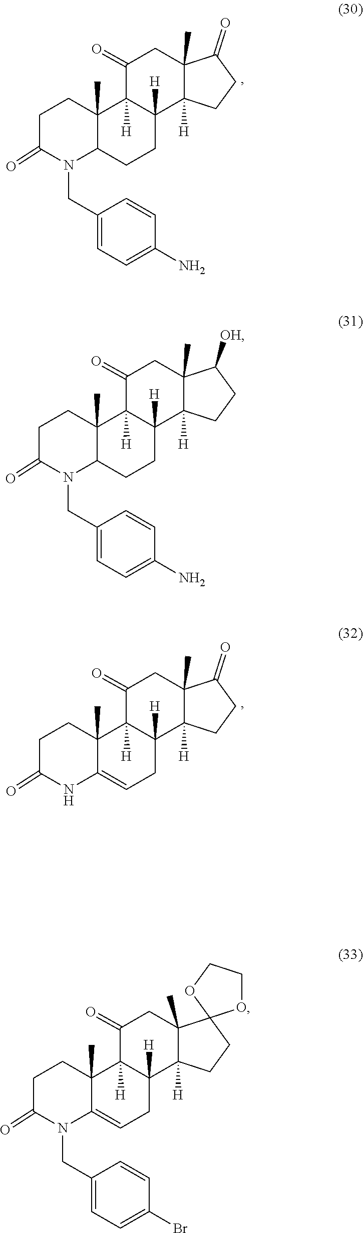

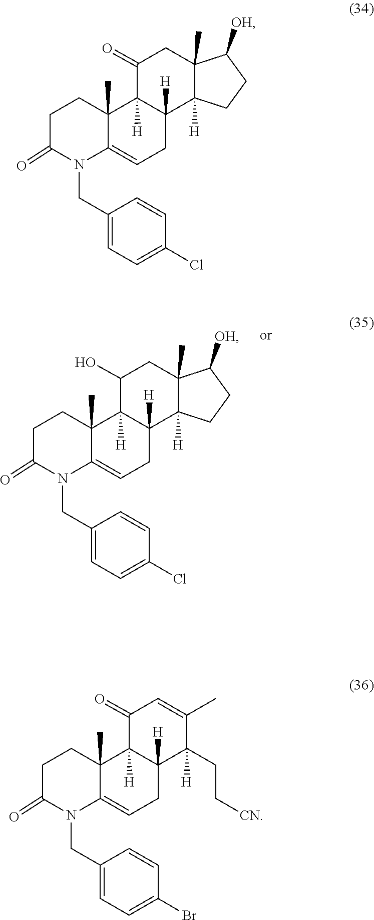

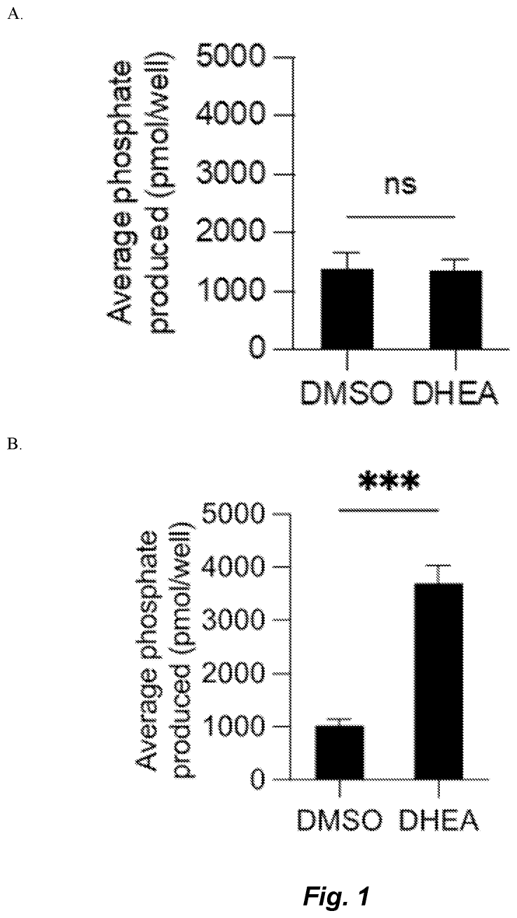

In vitro identification and validation of SULT2B1b inhibitors. To discover SULT2B1b inhibitors, an in vitro assay which is amenable to miniaturization and medium- to high-throughput screening was developed. There is a previously reported and commercial kit for a coupled chromogenic in vitro assay that measures general sulfotransferase protein specific activity (Scheme 1). However, this assay (Anal Biochem 2012, 423 (1), 86-92) has failed to distinguish samples with or without a sulfate acceptor molecule (dehydroepiandrosterone (DHEA), FIG. 1A). Optimization of assay conditions required heterologously expressed SULT2B1b protein (instead of commercially available SULT2B1b) as well as variations in protein concentration, PAPS concentration, DHEA concentration, reaction time, and presence of protein stabilization buffer. These modifications yielded an assay with significant differences (p=0.0002) between samples containing either DHEA or DMSO (FIG. 1B). With this assay now in hand, an in-house compound collection produced using the complexity-to-diversity natural product modification strategy was screened. This screening library was chosen due to its high prevalence of compounds generated from steroidal scaffolds similar to cholesterol, the natural substrate of SULT2B1b.

Scheme 1. In vitro assessment of compound IB:10:D. Assay schematic for the coupled chromogenic in vitro assay, originally developed to measure sulfotransferase enzyme specific activity. A sulfate acceptor molecule (here DHEA) and 3′-phosphoadenosine-5′-phosphosulfate (PAPS) are incubated with sulfotransferase protein of interest to form DHEA sulfate and 3′-phosphoadenosine-5′-phosphate (PAP). PAP is a substrate for inositol monophosphatase domain containing protein 1 (IMPAD1), which catalyzes the removal of the 3′ phosphate group to yield adenosine monophosphate (AMP) and one equivalent of inorganic phosphate. Inorganic phosphate levels are measured using a malachite green phosphate detection reagent.

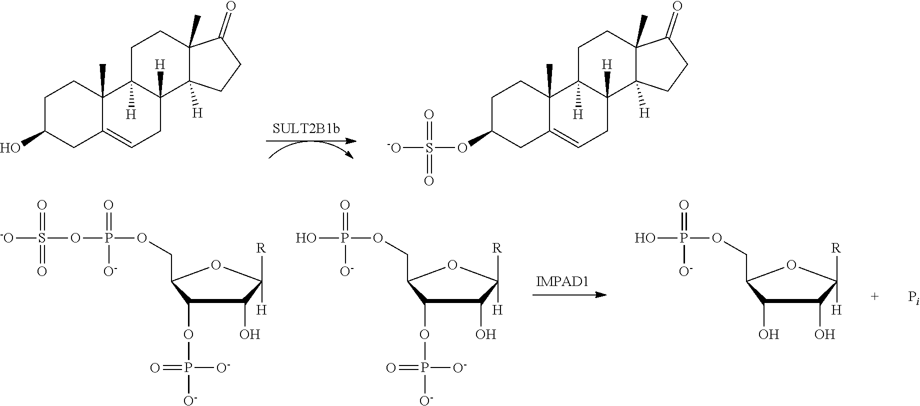

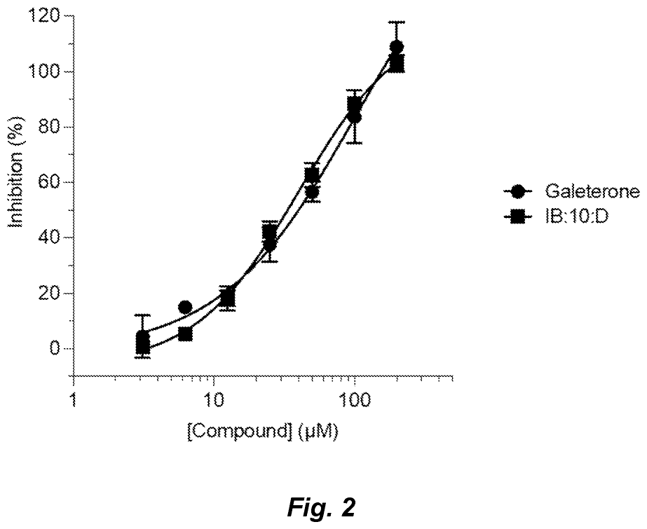

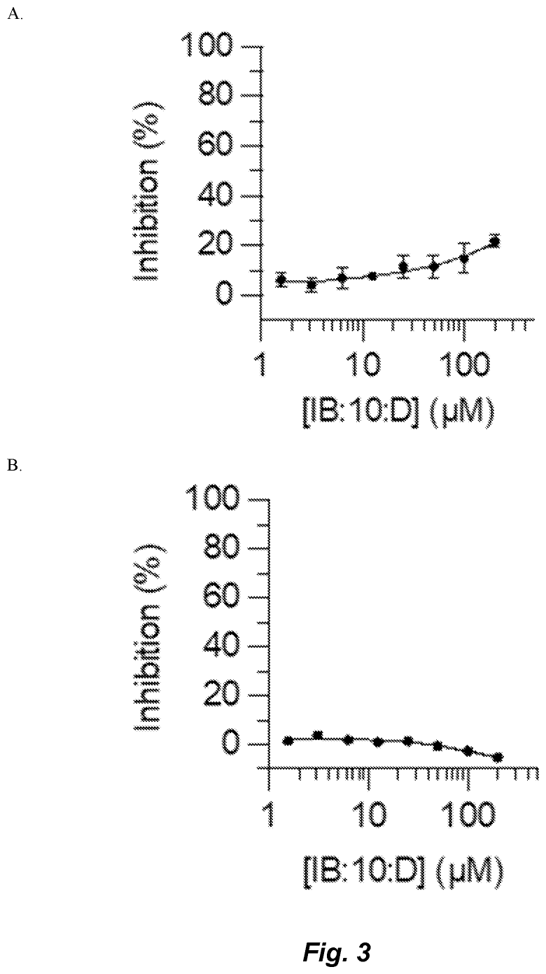

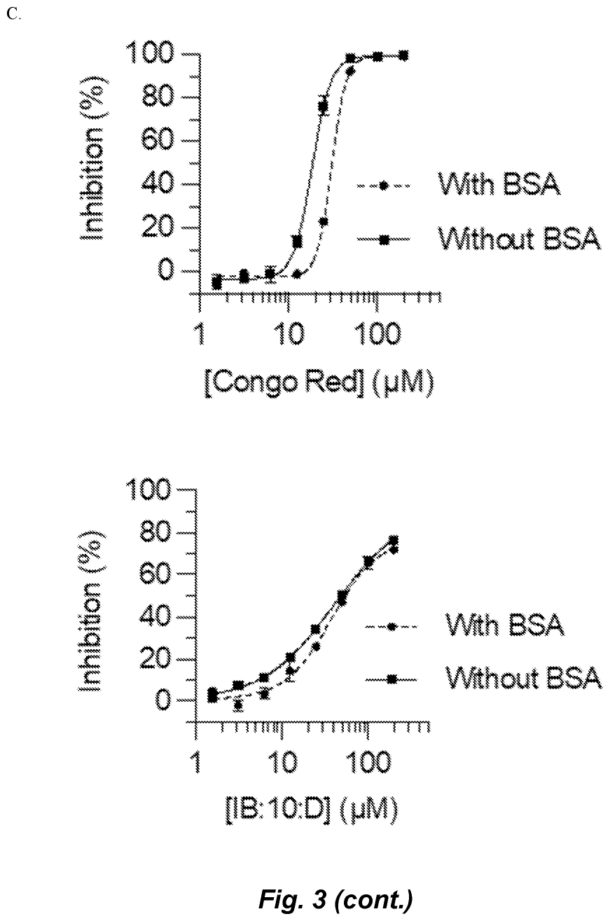

Compound IB:10:D (1, Table 1) was identified from an initial medium throughput screen with a percent inhibition of 48%. Dose-dependent inhibition of SULT2B1b by IB:10:D was confirmed using resynthesized compound in the coupled chromogenic in vitro assay, and its inhibition curve closely mirrored that of galeterone (2, Table 1) when subjected to identical assay conditions (FIG. 2 and Table 1). To confirm that IB:10:D activity in this coupled chromogenic assay is indeed the result of SULT2B1b inhibition and not the inhibition of the secondary phosphate-generating enzyme used in the assay, an LC-MS/MS SULT2B1b assay was developed to assess inhibition without the need for a secondary protein. In this assay, IB:10:D, as well as galeterone, inhibited SULT2B1b as measured by decreases in DHEA-sulfate levels (FIG. 3A). The level of inhibition of SULT2B1b by IB:10:D was found to be superior to galeterone in this LC-MS/MS assay. IB:10:D does not inhibit inositol monophosphatase domain-containing protein 1 (IMPAD1), the phosphatase used in the coupled chromogenic assay (FIG. 3B). IB:10:D also does not inhibit human carbonic anhydrase II (hCAII) at concentrations up to 200 μM (FIG. 3C); confirming that IB:10:D is likely not a non-specific inhibitor of in vitro enzymes. Enzyme inhibition by an aggregation-type mechanism was examined by inclusion of the decoy protein bovine serum albumin (BSA) in a dose response version of the in vitro LC-MS/MS assay, with IB:10:D showing a minimal shift in enzyme inhibition in the presence of BSA as opposed to the positive control molecule Congo Red (FIG. 3D). In total, these data indicate that IB:10:D inhibits SULT2B1b in a dose-dependent manner.

TABLE 1
Discovery of IB:10:D and comparison to previously identified SULT2B1b
inhibitor galeterone.
(1)

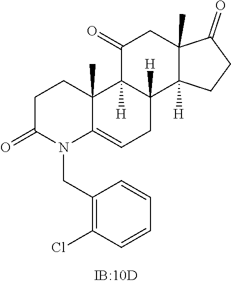

(2)

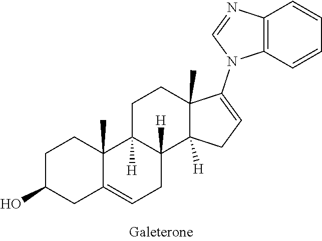

| Compound | IC₅₀ (μM) |
|---|---|
| Galeterone | 41 ± 7 |
| IB:10:D | 34 ± 4 |

To establish initial structure activity relationships (SAR) and possibly identify compounds that could increase SULT2B1b inhibition, a small panel of derivatives was synthesized and evaluated for their ability to inhibit SULT2B1b at a single concentration using an LC-MS/MS based assay. The results of this SAR screen are reported in Table 2. Modification of the aryl group from a para-chloro substituent to a para-bromo (compound 3/2-273) slightly increased inhibition of SULT2B1b. Substitution to a para-fluoro substituent (4) or removal of the aryl halide (5) significantly decreased SULT2B1b inhibition, albeit still maintaining near 50% inhibition. Removal of the aryl group completely and replacement with N-methyl substitution of the enamide moiety (compound 6/3-67) resulted in almost complete ablation of activity, suggesting the necessity for substitution at this position. Compound 6 (3-67)'s inactivity represented a structurally related negative control compound that was used for further experiments. Exploration of a meta (7) substitution pattern yielded a compound with similar SULT2B1b inhibition, however an ortho (8) substitution pattern led to decreased enzyme inhibition.

TABLE 2
Summary of IB:10:D derivatives and corresponding SULT2B1b
single point enzyme inhibition (see Chart 2).

| | Compound | | | | | | |
|---|---|---|---|---|---|---|---|
| | 1 | 2 | 3 | | | 6 | |
| | (IB:10:D) | (Galeterone) | (2-273) | 4 | 5 | (3-67) | 7 |
| Inhibition (%) | 67.8 ± 0.5 | 57.0 ± 0.7 | 72.8 ± 0.1 | 55 ± 2 | 48 ± 2 | 8.8 ± 0.4 | 69.8 ± 0.5 |

| | Compound | | | | | | |
|---|---|---|---|---|---|---|---|
| | 8 | 9 | 10 | 11 | 12 | 13 | 14 |
| Inhibition (%) | 46 ± 1 | 53 ± 1 | 45 ± 1 | 53 ± 1 | 52 ± 10 | 27 ± 2 | 48 ± 2 |

Enzyme inhibition determined at 100 μM compound using the LC-MS/MS based in vitro assay described in FIG. 3A. Data plotted as mean±s.e.m.; n=3 independent replicates.

Small modifications around the IB:10:D core including saturation at the C-11 position (9), and acetal formation at the C-17 position (10), resulted in decreased SULT2B1b inhibition. Introduction of nitrogen to the aryl ring in the form of a 4-bromopyridine (11) again decreased SULT2B1b inhibition, as did increasing size of the aryl substituent through introduction of a naphthyl group (12). Alkyl substitution of the enamide was also not fruitful, with both cyclopropyl (13) and cyclohexyl (14) substituents showing decreased SULT2B1b inhibitory activity. Ultimately, compounds IB:10:D and 3 (2-273) were chosen for further exploration in mammalian cell culture models due to their in vitro SULT2B1b inhibitory activity.

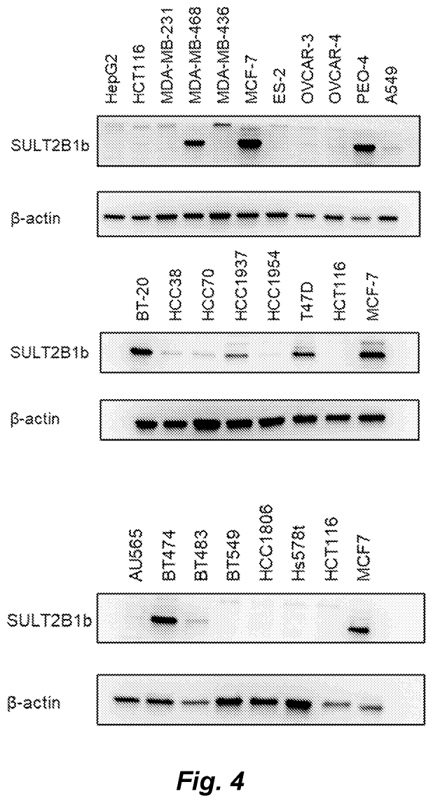

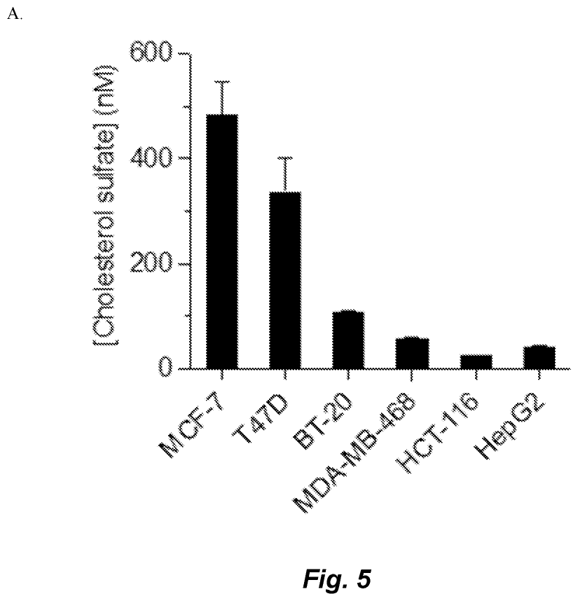

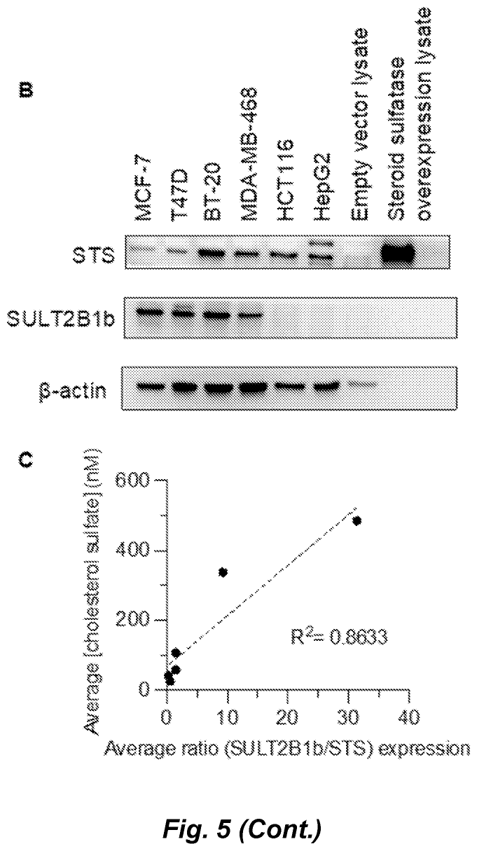

Assessment of compounds IB:O:D and 3 (2-273) in cell culture. Before compounds could be assessed for SULT2B1b inhibitory activity in cell culture, an appropriate cancer cell line with sufficient SULT2B1b expression and correspondingly high cholesterol sulfate levels was needed. A panel of cell lines was screened for SULT2B1b expression via western blot analysis (FIG. 4). Cell lines with high and low expression of SULT2B1b were then interrogated for their relative CS levels (FIG. 5A). MCF-7 and T47D, both strong expressors of SULT2B1b, showed high levels of cholesterol sulfate. Interestingly, some cell lines with robust SULT2B1b expression, such as BT-20 or MDA-MB-468, had low levels of CS, similar to levels seen in low SULT2B1b expressing cancer cell lines HCT-116 and HepG2. A hypothesis was formulated that this could be due to the sulfate ‘eraser’ activity of STS, known to remove the sulfate group of CS. Indeed, more in-depth exploration of these cancer cell lines revealed that STS expression was sufficient to lower overall CS levels in cells that express high levels of SULT2B1b (FIG. 5B). The ratio of SULT2B1b to STS expression showed good correlation (R²=0.8633) for cellular CS levels (FIG. 5C). MCF-7 and T47D cells possess high levels of SULT2B1b, relatively low levels of STS, and thus higher levels of CS. Conversely, while BT-20 and MDA-MB-468 also have high levels of SULT2B1b, both also have comparatively higher expression levels of STS and therefore low levels of CS.

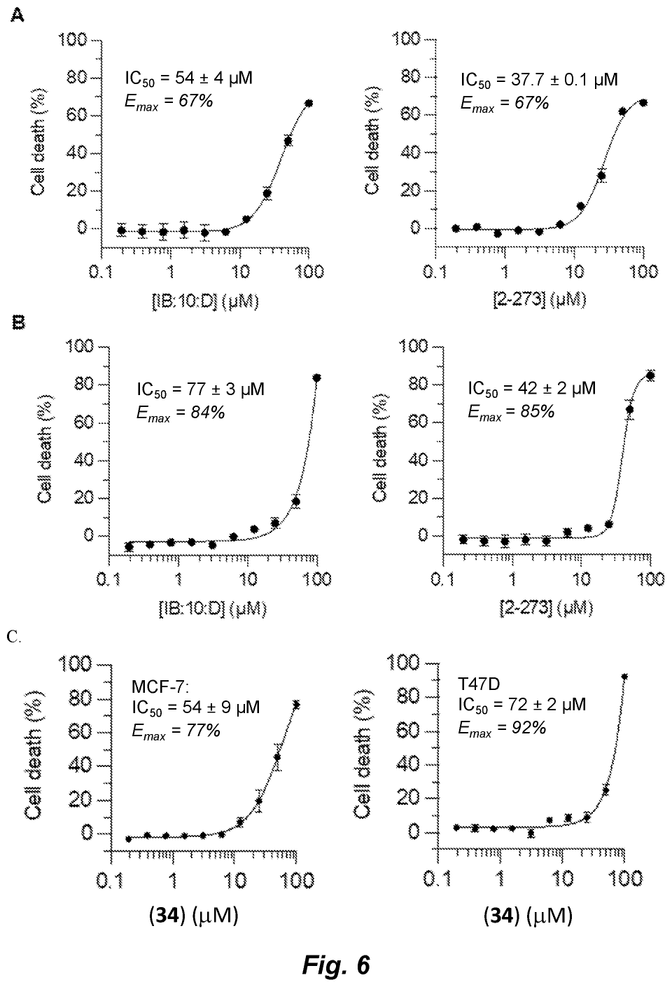

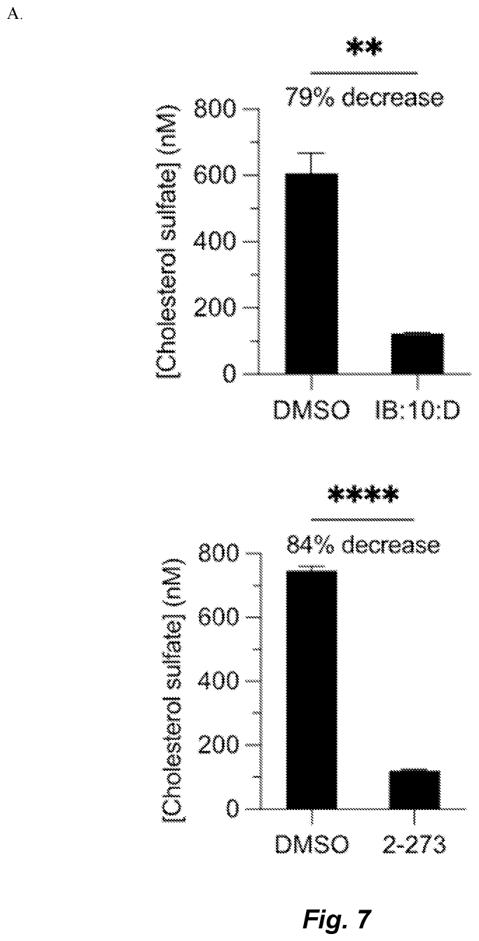

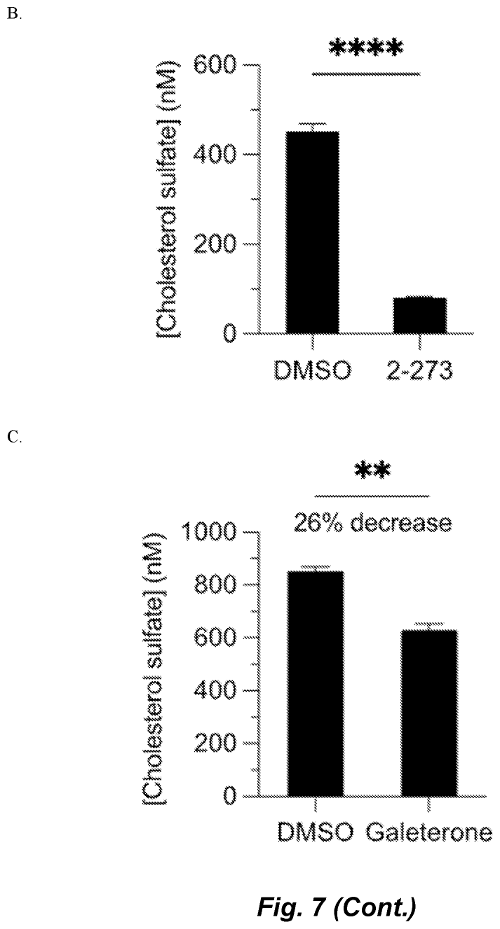

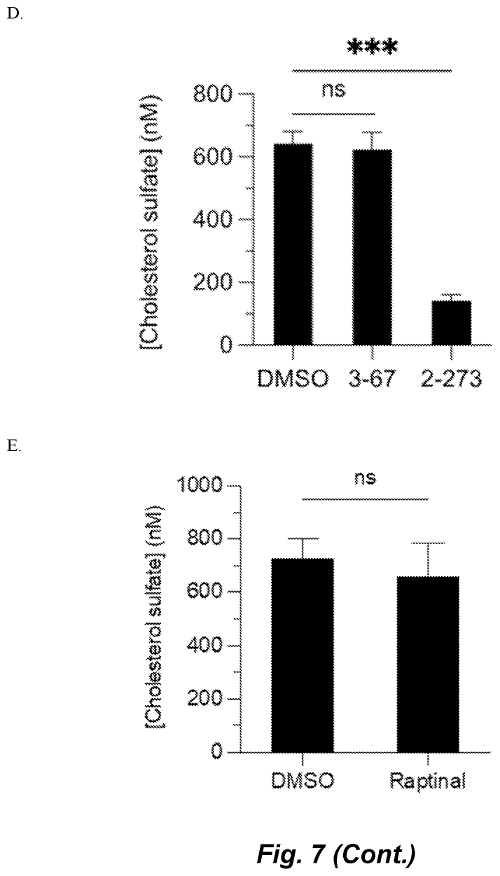

With two suitable cancer cell lines (MCF-7 and T47D) identified for assessing SULT2B1b and CS levels in cells, a goal was to test compounds IB:10:D and 2-273 in MCF-7 using a cell-based assay measuring levels of whole cell CS after compound treatment. Although the ultimate biological effects of inhibition of SULT2B1b are relatively underexplored, results collated from CRISPR or RNAi screens show that knockout or knockdown of SULT2B1 across cell lines is non-lethal, suggesting that inhibition of SULT2B1b should not be lethal. To ensure that IB:10:D and 2-273 were evaluated in whole cells at sub-lethal concentrations, cellular cytotoxicity was evaluated against MCF-7 and T47D cells at 72 hours (FIG. 6). A treatment concentration of 6.25 μM was found to be suitable for both IB:10:D and 2-273 in both cell lines. At this concentration, both compounds were found to significantly decrease whole cell CS levels in MCF-7 after 72-hour incubation (FIG. 7A), suggesting these compounds inhibit SULT2B1b in cells. Compound 2-273 was slightly better at decreasing whole cell CS levels, analogous to results seen in vitro (Table 2), justifying compound 2-273 as the molecule chosen for further study. Activity of 2-273 in cells was confirmed in T47D (FIG. 7B).

Galeterone was also evaluated in this assay in MCF-7, and while it did decrease CS levels, it was not to the same extent as either IB:10:D or 2-273 (FIG. 7C). When compared to compound 3-67 (inactive control), 2-273 again showed a significant (p=0.0003) reduction of whole cell CS levels, a result not seen in samples treated with 3-67 (p=0.788; FIG. 7D). To further confirm that the observed decrease in CS level was from SULT2B1b inhibition and not a function of the beginning stages of cell death or other general compound treatment effects, MCF-7 cells were treated with sub-lethal levels of the apoptosis inducer Raptinal and assessed the whole cell CS levels. There was no significant difference between DMSO or Raptinal treated samples (p=0.656; FIG. 7E). These cellular results (combined with in vitro work) are consistent with compound 2-273 inhibiting SULT2B1b in cells leading to specific modulation of CS levels.

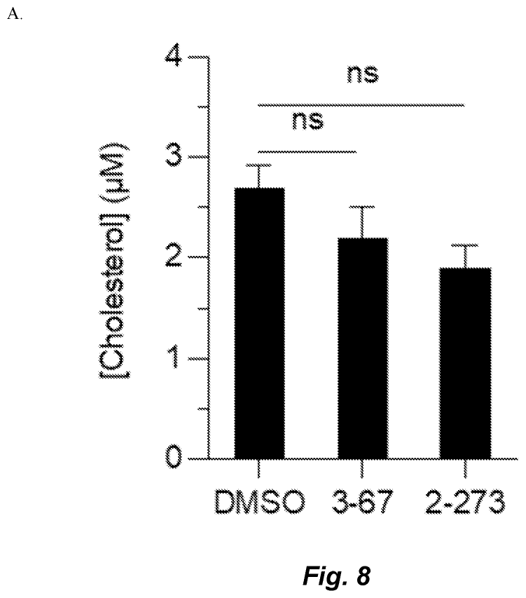

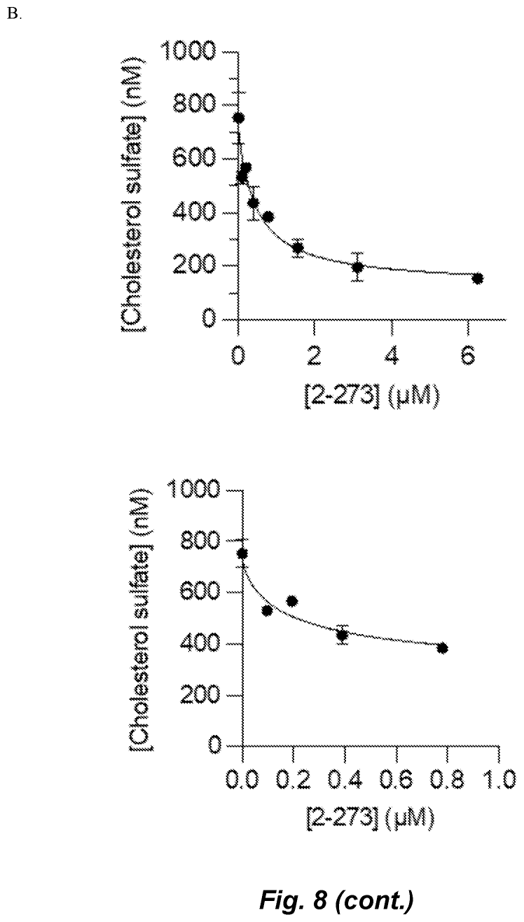

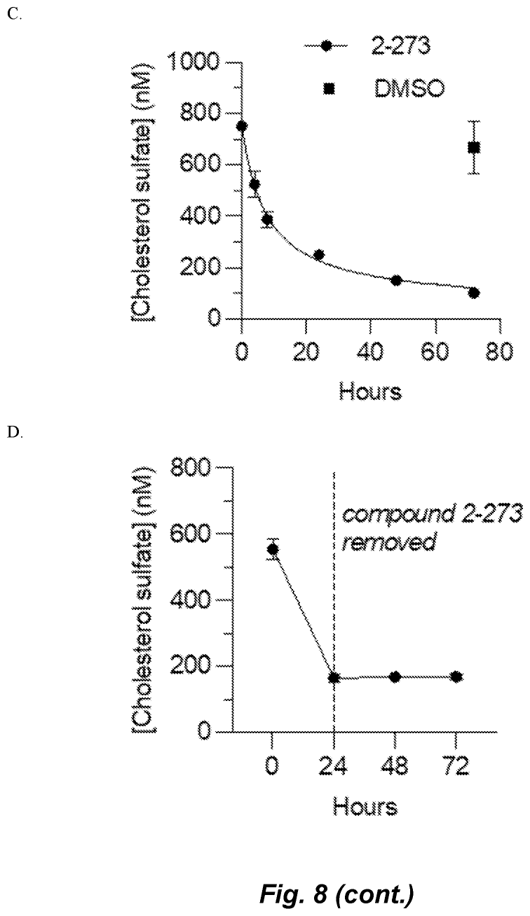

To further validate compound 2-273 as a chemical probe for SULT2B1b inhibition, assurance was needed that 2-273 was not affecting either upstream production of CS or downstream metabolism of CS, two mechanisms of action that could not be confirmed using in vitro assays. To confirm that 2-273 does not simply inhibit general cholesterol synthesis (which would ultimately decrease CS levels), a commercially available assay was employed to determine the levels of free cholesterol present in whole cells after treatment with DMSO, 2-273, or 3-67 (inactive control). There was no statistical difference (p=0.061 for 2-273, p=0.254 for 3-67) found between cholesterol levels treated with these three conditions (FIG. 8A). It remained possible that 2-273 treatment could lead to upregulation of STS and thus the observed CS level decreases. Western blot analysis showed that STS and SULT2B1b levels after 2-273 treatment were unchanged in MCF-7 or T47D after treatment with 6.25 μM 2-273 for 72 hours.

In an effort to further understand the cellular activity of probe 2-273, a whole cell dose response assay was performed which showed a decrease in CS levels with treatment concentrations as low as 0.097 μM 2-273 (FIG. 8B). This potency is discordant with the potency seen in vitro and may simply be the result of the contrived nature of the in vitro assay (e.g., high protein concentration). The timing of CS decreases was also interrogated using a time course assay that showed within 24 hours of constant compound treatment, the whole cell CS levels were significantly (p=0.00002) decreased and continue to decrease over 72 hours (FIG. 8C). To study the kinetics of CS level rebound post-compound treatment, cells were treated with 2-273 for 24 hours before media was replaced with compound-free media and CS levels determined at 24 and 48 hours after 2-273 removal (FIG. 8D). This experiment revealed that CS levels did not rebound up to 48 hours after compound removal.

Assessment of compound 2-273 in vivo. A chemical probe (compound 2-273) was now available for SULT2B1b inhibition that was sufficient for in vitro and in cell culture assays. Due to the role of SULT2B1b and CS in establishing an immunosuppressive tumor microenvironment, it was necessary to assess whether compound 2-273 was competent as an in vivo chemical probe. To assess a variety of hypotheses surrounding SULT2B1b/CS, again a suitable cell line needed to be found. Ideally, a murine cancer cell line with high SULT2B1b and corresponding CS levels was desirable, as it would allow for a syngeneic model system and a full study of the effect of SULT2B1b inhibition on the interaction between the immune system and a tumor. Unfortunately, attempts to identify a murine cancer cell line that had robust SULT2B1b expression and CS levels were unsuccessful. Ultimately, a tumorigenic human cancer cell line was chosen to move forward with. One advantage of using a human cell line is that it removes uncertainty surrounding whether 2-273 would inhibit murine SULT2B1b, which has only a 76% sequence homology with the human enzyme.

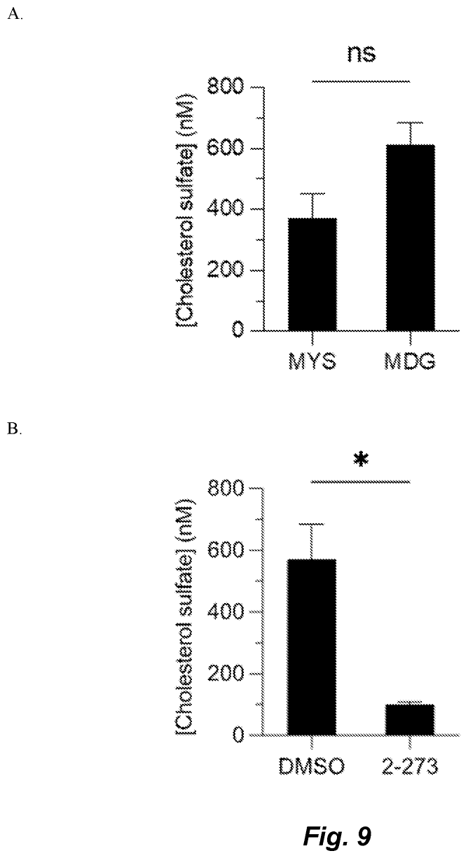

The MCF-7 cell line would be an ideal candidate, but in vivo models using this estrogen-dependent cell line require additional implantation of an estrogen pellet to encourage tumor growth. However, extensive research on the estrogen receptor alpha in breast cancers has led to the construction of MCF-7 cells with mutations in the estrogen receptor alpha that allow for estrogen independent cell growth. These estrogen receptor alpha mutant MCF-7 cells would allow us to study SULT2B1b inhibition in vivo without the experimental complications of estrogen dependence. Two MCF-7 cell lines were selected that harbor either a Y537S mutation (MYS cells) or mutation D538G (MDG cells) in estrogen receptor alpha to confirm their SULT2B1b expression and CS levels (FIG. 9A). MDG possessed levels that were comparable what had been observed in the MCF-7 cell line. The ability of compound 2-273 to decrease CS levels in MDG was confirmed (FIG. 9B), allowing for the selection of MDG as a cancer cell line to use for in vivo explorations.

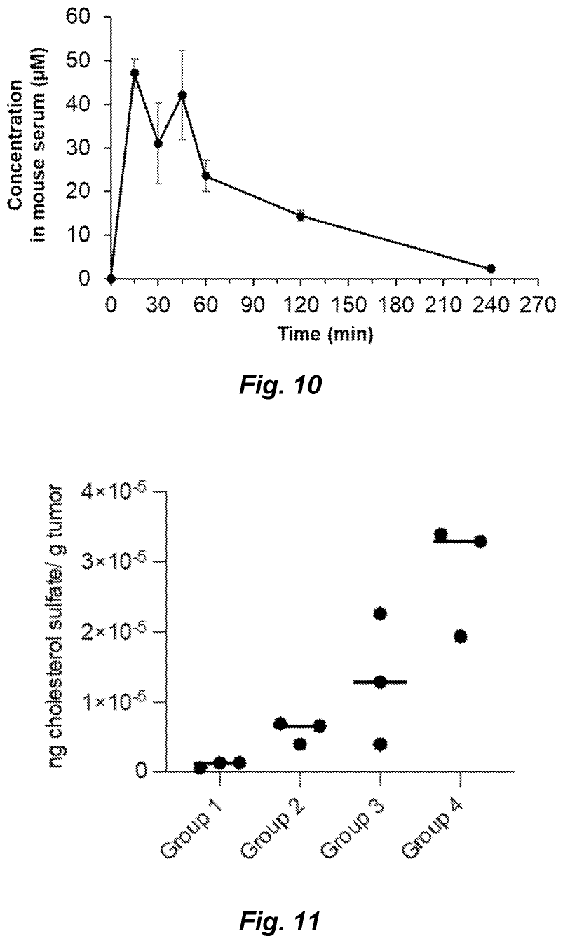

Initial studies showed that 2-273 is tolerated in mice up to 200 mg/kg when dosed intraperitoneally (IP) in a single injection. Probe 2-273 treatment is not tolerated when dosed at 200 mg/kg IP twice daily for multiple days. Therefore, a dosing strategy of 150 mg/kg IP once daily to study 2-273 in vivo was selected. Pharmacokinetic (PK) analysis of 2-273 when dosed at 150 mg/kg IP showed sufficiently high levels of compound detectable in the serum, with concentrations reaching up to almost 50 μM, well-above those needed for SULT2B1b inhibition activity (FIG. 10 and Table 3). Thus, 2-273's PK and tolerability are sufficient for studying SULT2B1b in vivo and positions 2-273 as a chemical probe that can be used in vitro, in cells, and in vivo.

TABLE 3
Pharmacokinetic Analysis of Compound (3) Shows High
Concentrations in the Serum.

| Parameter | Units | Estimate |
|---|---|---|
| AUC | min*ng/ml | 2208398.7 |
| t_{1/2} | min | 77.21993 |
| Cₘₐₓ | ng/ml | 19825.227 |
| MRT | min | 111.39336 |
| CL | mL/min/kg | 90.563358 |

Calculated pharmacokinetic parameters: AUC = area under the curve, t½ = half-life, Cmax = maximum concentration, MRT = mean residual time, CL = clearance rate.

### Pharmaceutical Formulations

The compounds described herein can be used to prepare therapeutic pharmaceutical compositions, for example, by combining the compounds with a pharmaceutically acceptable diluent, excipient, or carrier. The compounds may be added to a carrier in the form of a salt or solvate. For example, in cases where compounds are sufficiently basic or acidic to form stable nontoxic acid or base salts, administration of the compounds as salts may be appropriate. Examples of pharmaceutically acceptable salts are organic acid addition salts formed with acids that form a physiologically acceptable anion, for example, tosylate, methanesulfonate, acetate, citrate, malonate, tartrate, succinate, benzoate, ascorbate, α-ketoglutarate, and β-glycerophosphate. Suitable inorganic salts may also be formed, including hydrochloride, halide, sulfate, nitrate, bicarbonate, and carbonate salts.

Pharmaceutically acceptable salts may be obtained using standard procedures well known in the art, for example by reacting a sufficiently basic compound such as an amine with a suitable acid to provide a physiologically acceptable ionic compound. Alkali metal (for example, sodium, potassium or lithium) or alkaline earth metal (for example, calcium) salts of carboxylic acids can also be prepared by analogous methods.

The compounds of the formulas described herein can be formulated as pharmaceutical compositions and administered to a mammalian host, such as a human patient, in a variety of forms. The forms can be specifically adapted to a chosen route of administration, e.g., oral or parenteral administration, by intravenous, intramuscular, topical or subcutaneous routes.

The compounds described herein may be systemically administered in combination with a pharmaceutically acceptable vehicle, such as an inert diluent or an assimilable edible carrier. For oral administration, compounds can be enclosed in hard or soft shell gelatin capsules, compressed into tablets, or incorporated directly into the food of a patient's diet. Compounds may also be combined with one or more excipients and used in the form of ingestible tablets, buccal tablets, troches, capsules, elixirs, suspensions, syrups, wafers, and the like. Such compositions and preparations typically contain at least 0.1% of active compound. The percentage of the compositions and preparations can vary and may conveniently be from about 0.5% to about 60%, about 1% to about 25%, or about 2% to about 10%, of the weight of a given unit dosage form. The amount of active compound in such therapeutically useful compositions can be such that an effective dosage level can be obtained.

The tablets, troches, pills, capsules, and the like may also contain one or more of the following: binders such as gum tragacanth, acacia, corn starch or gelatin; excipients such as dicalcium phosphate; a disintegrating agent such as corn starch, potato starch, alginic acid and the like; and a lubricant such as magnesium stearate. A sweetening agent such as sucrose, fructose, lactose or aspartame; or a flavoring agent such as peppermint, oil of wintergreen, or cherry flavoring, may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propyl parabens as preservatives, a dye and flavoring such as cherry or orange flavor. Any material used in preparing any unit dosage form should be pharmaceutically acceptable and substantially non-toxic in the amounts employed. In addition, the active compound may be incorporated into sustained-release preparations and devices.

The active compound may be administered intravenously or intraperitoneally by infusion or injection. Solutions of the active compound or its salts can be prepared in water, optionally mixed with a nontoxic surfactant. Dispersions can be prepared in glycerol, liquid polyethylene glycols, triacetin, or mixtures thereof, or in a pharmaceutically acceptable oil. Under ordinary conditions of storage and use, preparations may contain a preservative to prevent the growth of microorganisms.

Pharmaceutical dosage forms suitable for injection or infusion can include sterile aqueous solutions, dispersions, or sterile powders comprising the active ingredient adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions, or by the use of surfactants. The prevention of the action of microorganisms can be brought about by various antibacterial and/or antifungal agents, for example, parabens, chlorobutanol, phenol, sorbic acid, thimerosal, and the like. In many cases, it will be preferable to include isotonic agents, for example, sugars, buffers, or sodium chloride. Prolonged absorption of the injectable compositions can be brought about by agents delaying absorption, for example, aluminum monostearate and/or gelatin.

Sterile injectable solutions can be prepared by incorporating the active compound in the required amount in the appropriate solvent with various other ingredients enumerated above, as required, optionally followed by filter sterilization. In the case of sterile powders for the preparation of sterile injectable solutions, methods of preparation can include vacuum drying and freeze drying techniques, which yield a powder of the active ingredient plus any additional desired ingredient present in the solution.

For topical administration, compounds may be applied in pure form, e.g., when they are liquids. However, it will generally be desirable to administer the active agent to the skin as a composition or formulation, for example, in combination with a dermatologically acceptable carrier, which may be a solid, a liquid, a gel, or the like.

Useful solid carriers include finely divided solids such as talc, clay, microcrystalline cellulose, silica, alumina, and the like. Useful liquid carriers include water, dimethyl sulfoxide (DMSO), alcohols, glycols, or water-alcohol/glycol blends, in which a compound can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using a pump-type or aerosol sprayer.

Thickeners such as synthetic polymers, fatty acids, fatty acid salts and esters, fatty alcohols, modified celluloses, or modified mineral materials can also be employed with liquid carriers to form spreadable pastes, gels, ointments, soaps, and the like, for application directly to the skin of the user.

Examples of dermatological compositions for delivering active agents to the skin are known to the art; for example, see U.S. Pat. No. 4,992,478 (Geria), U.S. Pat. No. 4,820,508 (Wortzman), U.S. Pat. No. 4,608,392 (Jacquet et al.), and U.S. Pat. No. 4,559,157 (Smith et al.). Such dermatological compositions can be used in combinations with the compounds described herein where an ingredient of such compositions can optionally be replaced by a compound described herein, or a compound described herein can be added to the composition.

Useful dosages of the compounds or compositions described herein can be determined by comparing their in vitro activity, and in vivo activity in animal models. Methods for the extrapolation of effective dosages in mice, and other animals, to humans are known to the art; for example, see U.S. Pat. No. 4,938,949 (Borch et al.). The amount of a compound, or an active salt or derivative thereof, required for use in treatment will vary not only with the particular compound or salt selected but also with the route of administration, the nature of the condition being treated, and the age and condition of the patient, and will be ultimately at the discretion of an attendant physician or clinician.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The compound is conveniently formulated in unit dosage form; for example, containing 5 to 1000 mg, conveniently 10 to 750 mg, most conveniently, 50 to 500 mg of active ingredient per unit dosage form. In one embodiment, the invention provides a composition comprising a compound of the invention formulated in such a unit dosage form.

The compound can be conveniently administered in a unit dosage form, for example, containing 5 to 1000 mg/m², conveniently 10 to 750 mg/m², most conveniently, 50 to 500 mg/m² of active ingredient per unit dosage form. The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

The invention provides therapeutic methods of treating cancer in a mammal, which involve administering to a mammal having cancer an effective amount of a compound or composition described herein. A mammal includes a primate, human, rodent, canine, feline, bovine, ovine, equine, swine, caprine, bovine and the like. Cancer refers to any various type of malignant neoplasm, for example, colon cancer, gastric cancer, breast cancer, hetpatocellular cancer, melanoma and leukemia, and in general is characterized by an undesirable cellular proliferation, e.g., unregulated growth, lack of differentiation, local tissue invasion, and metastasis.

The ability of a compound of the invention to treat cancer may be determined by using assays well known to the art. For example, the design of treatment protocols, toxicity evaluation, data analysis, quantification of tumor cell kill, and the biological significance of the use of transplantable tumor screens are known.

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### Examples

### Example 1. Assay Development

Reagents. IMPAD1 (catalog number 7028-PD-050) and PAPS (catalog number ES019) were purchased from R&D systems. DHEA (catalog number D4000) was purchased from Sigma Aldrich at ≥99% purity. Galeterone (catalog number HY-70006) was purchased from MedChemExpress at ≥99% purity. Adenosine 3′,5′-diphosphate disodium salt (PAP) was purchased from Sigma Aldrich at ≥96% purity. Bovine serum albumin (catalog number 100-10) was purchased from Lee BioSolutions. Rabbit anti-SULT2B1 antibody (catalog number ab254617) and rabbit anti-STS antibody (catalog number ab233233) were purchased from Abcam. Rabbit HRP-conjugated anti-beta-actin (catalog number 5125S), HRP-conjugated goat anti-rabbit (catalog number 7074S), mouse anti-histidine tag antibody (catalog number 2366S), and HRP-conjugated horse anti-mouse antibody (catalog number 7076S) were purchased from Cell Signaling Technology. Cholesterol/Cholesterol Ester-Glo Assay Kit was purchased from Promega. All other chemicals were purchased from Sigma-Aldrich or Fisher Scientific in the highest available purity.

Buffer and Media Compositions. Terrific Broth: 1.2% tryptone (12 g/L), 2.4% yeast extract (24 g/L), 0.5% glycerol (5 g/L). If making 1 L of media, dissolve 12 g tryptone, 24 g yeast extract, and 5 g glycerol in 900 mL MilliQ H₂O. Autoclave 15 min at 121° C. Add 10× terrific broth salts (prepared separately) to media at a ratio of 1 mL 10× terrific broth salts:9 mL terrific broth. 10× Terrific Broth Salts: 0.17 M KH₂PO₄ (23.1 g/L), 0.72 M K₂HPO₄ (125.4 g/L). Dissolve potassium salts in MilliQ H₂O, then autoclave for 15 min at 121° C. SULT2B1b lysis buffer: 10 mM Tris, 150 mM NaCl, 1 mM TCEP, 1 mM PMSF, 10 μL Triton-X 100, 1 μg/mL leupeptin, 1 μg/mL pepstatin A, 2 μg/mL aprotinin, pH 8.0. SULT2B1b equilibration buffer: 10 mM Tris, 150 mM NaCl, 1 mM TCEP, pH 8.0. SULT2B1b Purification Low Salt Wash buffer: 10 mM Tris, 20 mM imidazole, 150 mM NaCl, 1 μg/mL leupeptin, 1 μg/mL pepstatin A, 2 μg/mL aprotinin, pH 8.0. SULT2B1b Purification High Salt Wash buffer: 10 mM Tris, 20 mM imidazole, 500 mM NaCl, 1 μg/mL leupeptin, 1 μg/mL pepstatin A, 2 μg/mL aprotinin, pH 8.0. SULT2B1b Purification Elution buffer: 10 mM Tris, 50-200 mM imidazole, 150 mM NaCl, 1 μg/mL leupeptin, 1 μg/mL pepstatin A, 2 μg/mL aprotinin, pH 8.0. SULT2B1b dialysis and protein storage buffer: 25 mM Tris, 150 mM NaCl, 5 mM TCEP, 50% glycerol, pH 7.5. Malachite green solution A: 28 mM ammonium molybdate, 2.1 M H₂SO₄. Malachite green solution B: 0.59 mM malachite green oxalate, 0.35% polyvinyl alcohol. SULT2B1b 1X activity assay buffer: 50 mM Tris, 15 mM MgCl₂, pH 7.5. hCAII assay buffer: 50 mM MOPS, 33 mM Na₂SO₄, 1 mM EDTA, pH 7.5.

Optimized Coupled Chromogenic SULT2B1b Activity Assay. For 96-well Format: Working concentrations of IMPAD1 (0.1 mg/mL in 1× activity assay buffer), PAPS (1 mM in 1× activity assay buffer), and DHEA (2.5 mM in DMSO) were prepared. Two technical replicates were assayed for each sample. Reaction mixes were prepared by mixing 10 μL PAPS stock solution, 10 μL IMPAD1 stock solution, 4 μL 2.5 mM DHEA or DMSO, and 16 μL activity assay buffer to make 50 μL reaction mix. Amount of reaction mix was scaled accordingly to number of samples assayed. 25 μL of reaction mix was dispensed to each well. SULT2B1b was diluted to 1.5 μg/μL in protein storage buffer then down to 0.8 μg/μL in 1X activity assay buffer. 25 μL of 0.8 μg/μL SULT2B1b was added to each well. The reaction was incubated at 37° C. for 45 minutes, then quenched by addition of 30 μL of malachite green solution A, followed by addition of 30 μL of malachite green solution B. The plate was left at room temperature for 20 minutes for color stabilization, then absorbance was read at 620 nm. IMPAD1 activity control was 10 μL of 0.1 mg/mL IMPAD1 mixed with 40 μL activity assay buffer. 25 μL of the IMPAD1 dilution was added to 25 μL of 0.1 mM PAP.

Phosphate Standard Curve for 96-well Format: 40 μL of 1 mM KH₂PO₄ was diluted with 360 μL SULT2B1b activity assay buffer. 2-fold serial dilutions were performed to get 7 total phosphate standards ranging from 100 μM (initial dilution) to 1.56 μM. 50 μL of each standard was added to each well, in 2 technical replicates, along with one well that contained only 50 μL of SULT2B1b activity assay buffer (0 μM). The phosphate standard curve ranged from 5000 pmol/well phosphate (100 μM standard) to 78 pmol/well phosphate (1.56 μM standard).

For 384 well format: Working concentrations of IMPAD1 (0.1 mg/mL in activity assay buffer), PAPS (1 mM in activity assay buffer), and DHEA (2.5 mM in DMSO) were prepared. Two technical replicates were assayed for each sample. Reaction mixes were prepared by mixing 12 μL PAPS stock solution, 6 μL IMPAD1 stock solution, 2.4 μL 2.5 mM DHEA or DMSO, and 9.6 μL activity assay buffer to make 30 μL reaction mix. Amount of reaction mix was scaled accordingly to number of samples assayed. 15 μL of reaction mix was dispensed to each well. SULT2B1b was diluted to 1.5 μg/μL in protein storage buffer then down to 0.8 μg/μL in activity assay buffer. 15 μL of 0.8 μg/μL SULT2B1b was added to each well. The reaction was incubated at 37° C. for 45 minutes, then quenched by addition of 15 μL of malachite green solution A, followed by addition of 15 μL of malachite green solution B. The plate was left at room temperature for 20 minutes for color stabilization, then absorbance was read at 620 nm. IMPAD1 activity control was 6 μL of 0.1 mg/mL IMPAD1 mixed with 40 μL activity assay buffer. 15 μL of the IMPAD1 dilution was added to 15 μL of 0.1 mM PAP.

Phosphate Standard Curve for 384-well Format: 160 μL of 1 mM KH₂PO₄ was diluted with 1.04 mL SULT2B1b activity assay buffer. 2-fold serial dilutions were performed to get 5 total phosphate standards ranging from 133 μM (initial dilution) to 8.3 μM. For a second set of standards, 120 μL of 1 mM KH₂PO₄ was diluted with 1.08 mL SULT2B1b activity assay buffer. 2-fold serial dilutions were performed to get 2 total phosphate standards ranging from 100 μM (initial dilution) to 50 μM. 30 μL of each standard was added to each well, in 2 technical replicates, along with one well that contained only 30 μL of SULT2B1b activity assay buffer (0 μM). The phosphate standard curve ranged from 4000 pmol/well phosphate (133 μM standard) to 250 pmol/well phosphate (8.3 μM standard).

Coupled Chromogenic SULT2B1b Inhibition Assay. For 96-well Format: Working stock solutions of IMPAD1 (0.1 mg/mL in 1× activity assay buffer), PAPS (1 mM in 1X activity assay buffer), compound of interest (5 mM in DMSO) and DHEA (5 mM in DMSO) were prepared. Two technical replicates were assayed for each sample. Reaction mixes were prepared by mixing 10 μL PAPS stock solution, 10 μL IMPAD1 stock solution, 2 μL 5 mM DHEA, 2 μL 5 mM compound of interest or DMSO, and 16 μL activity assay buffer to make 50 μL reaction mix. Amount of reaction mix was scaled accordingly to number of samples assayed. 25 μL of reaction mix was dispensed to each well. SULT2B1b was diluted to 1.5 μg/μL in protein storage buffer then down to 0.8 μg/μL in 1X activity assay buffer. 25 μL of 0.8 μg/μL SULT2B1b was added to each well. The reaction was incubated at 37° C. for 45 minutes, then quenched by addition of 30 μL of malachite green solution A, followed by addition of 30 μL of malachite green solution B. The plate was left at room temperature for 20 minutes for color stabilization, then absorbance was read at 620 nm. IMPAD1 activity control was 10 μL of 0.1 mg/mL IMPAD1 mixed with 40 μL activity assay buffer. 25 μL of the IMPAD1 dilution was added to 25 μL of 0.1 mM PAP. Phosphate standard curve generation was identical to that described above. Controls: Reaction mix made with DHEA and no compound of interest (DHEA control) and reaction mix made with DMSO, no DHEA or compound of interest (DMSO control).

Percent inhibition was calculated as: 100−[100×[(Phosphate produced per well_{average,compound of interest}−Phosphate produced per well_{average, DMSO})/(Phosphate produced per well_{average, DHEA}−Phosphate produced per well_{average, DMSO})]].

For 384 well format: Working concentrations of IMPAD1 (0.1 mg/mL in activity assay buffer), PAPS (1 mM in activity assay buffer), compound of interest (5 mM in DMSO) and DHEA (5 mM in DMSO) were prepared. Two technical replicates were assayed for each sample. Reaction mixes were prepared by mixing 12 μL PAPS stock solution, 6 μL IMPAD1 stock solution, 1.2 μL 5 mM DHEA, 1.2 μL 5 mM compound of interest or DMSO, and 9.6 μL activity assay buffer to make 30 μL reaction mix. Amount of reaction mix was scaled accordingly to number of samples assayed. 15 μL of reaction mix was dispensed to each well. SULT2B1b was diluted to 1.5 μg/μL in protein storage buffer then down to 0.8 μg/μL in activity assay buffer. 15 μL of 0.8 μg/μL sULT2B1b was added to each well. The reaction was incubated at 37° C. for 45 minutes, then quenched by addition of 15 μL of malachite green solution A, followed by addition of 15 μL of malachite green solution B. The plate was left at room temperature for 20 minutes for color stabilization, then absorbance was read at 620 nm. IMPAD1 activity control was 6 μL of 0.1 mg/mL IMPAD1 mixed with 40 μL activity assay buffer. 15 μL of the IMPAD1 dilution was added to 15 μL of 0.1 mM PAP. Phosphate standard curve generation was identical to that described above. Controls: Reaction mix made with DHEA and no compound of interest (DHEA control) and reaction mix made with DMSO, no DHEA or compound of interest (DMSO control).

Percent inhibition was calculated as: 100−[100×[(Phosphate produced per well_{average, compound of interest}−Phosphate produced per well_{average, DMSO})/(Phosphate produced per well_{average, DHEA}−Phosphate produced per well_{average, DMSO})]].

Expression and Purification of human SULT2B1b. Human SULT2B1b was cloned into either a pET-19b (N-terminal His6-tag) or pQE-60 (C-terminal His6-tag) plasmid and were obtained from Genscript Biotech. The plasmid was then transformed into chemically competent Rosetta 2 (DE3) or NiCo21 (DE3) E. coli cells. Overnight culture (1 mL) in LB supplemented with 100 μg/mL ampicillin and 20 μg/mL chloramphenicol for Rosetta 2, or 100 μg/mL ampicillin only for NiCo21 was diluted into 50 mL Terrific Broth (100 μg/mL ampicillin and 20 μg/mL chloramphenicol for Rosetta 2, or 100 μg/mL ampicillin only for NiCo21) was inoculated with 1 mL of overnight culture. The culture was grown at 37° C., 250 rpm to mid-log phase (OD600=0.5-0.6). Protein expression was induced with 0.5 mM IPTG for 8 h and harvested. Cultures were harvested by centrifugation at 3220 xg, 4° C., 30 min. Cell pellets were stored at −80° C. until purification. Frozen cell pellets were thawed on ice and resuspended in 1.5 mL SULT2B1b lysis buffer. Cells were lysed by sonication on ice (30%, 10 s pulse, 20 s rest, 5 min total). Lysate was clarified by centrifugation (14,100 xg, 1 h, 4° C.). Supernatant was incubated with 1 mL Ni-NTA agarose (pre-equilibrated with SULT2B1b equilibration buffer) for 1 h at 4° C. with gentle rocking. Agarose-containing supernatant was transferred to column and flow through was discarded. Column was washed with 7 mL low salt wash buffer, 7 mL high salt wash buffer, then another 7 mL low salt wash buffer. Protein was eluted with 7 mL 50-200 mM imidazole containing elution buffers. Fractions containing protein were identified by SDS-PAGE, concentrated with a 30 kDa molecular weight cutoff spin column, and subjected to dialysis against SULT2B1b storage buffer.

LC-MS/MS Based In Vitro Assay. Stock solutions of DHEA (5 mM in DMSO) and compound of interest (5 mM in DMSO) were prepared. For each 60 μL reaction, to a 1.5 mL Eppendorf tube was added 1.2 μL of 5 mM DHEA, 1.2 μL 5 mM compound of interest (for 100 μM final compound concentration) or DMSO, 0.9 μL 6.6 mM PAPS, and 26.7 μL 1× assay buffer. SULT2B1b was diluted to 1.5 μg/μL in protein storage buffer then down to 0.8 μg/μL in 1× activity assay buffer. 30 μL of 0.8 μg/μL SULT2B1b was added to each reaction. Reactions were incubated for 2 hours at 37° C. with shaking at 250 rpm. To quench, 30 μL reaction mix was added to 30 μL ice cold 0.5% HCl in MeCN, then centrifuged at 13,000 xg at room temperature for 3 min. 50 μL supernatant was transferred to an empty Eppendorf tube and submitted for LC-MS/MS quantification (UIUC Metabolomics Center), along with 5 mM DHEA sulfate standard. Controls: Reaction mix made with DHEA and DMSO in place of compound of interest (DHEA control).

Percent inhibition was calculated as: 100−[100×(DHEA-sulfate produced_{compound of interest}/DHEA sulfate produced_{DHEA})].

IMPAD1 Assay. Working stock solutions of IMPAD1 (0.1 mg/mL in 1× activity assay buffer), adenosine 3′,5′-diphosphate (PAP; dissolved first in DMSO to make stock solution then diluted to 200 μM in 1× assay buffer), and compound of interest in DMSO were prepared. Two technical replicates were assayed for each sample. Reaction mixes were prepared by mixing 6 μL IMPAD1 stock solution, 2.4 μL compound of interest stock or DMSO, 21.6 μL 1× assay buffer to make 30 μL reaction mix. Amount of reaction mix was scaled accordingly to number of samples assayed. 15 μL of reaction mix was dispensed to each well (384-well plate). 15 μL 200 μM PAP stock solution was added to each well. The reaction was incubated at 37° C. for 45 minutes, then quenched by addition of 15 μL of malachite green solution A, followed by addition of 15 μL of malachite green solution B. The plate was left at room temperature for 20 minutes for color stabilization, then absorbance was read at 620 nm. Phosphate standard curve generation was identical to that described under “Optimized Coupled Chromogenic SULT2B1b Activity Assay”, 384-well format. Controls: Reaction mix made with PAP and no compound of interest (IMPAD1 activity control), and reaction mix made with PAP, no IMPAD1 or compound of interest (PAP control).

Percent inhibition was calculated as: 100−[100×[(Phosphate produced per well_{average, compound of interest}−Phosphate produced per well_{average, PAP})/(Phosphate produced per well_{average, IMPAD1}−Phosphate produced per well_{average, PAP})]].

Human Carbonic Anhydrase II Assay. Stock solutions of compound of interest (in DMSO), acetazolamide (10 μM in DMSO), hCAII solution (65 μL of 33 μM stock to 1.935 mL hCAII assay buffer to make 1 μM hCAII solution), and 4-nitrophenylacetate (4-NPA; 60 μL of 100 mM DMSO stock to 3 mL hCAII assay buffer to make 2 mM 4-NPA solution). To the wells of a 384-well clear bottom plate were added 1 μL compound of interest or 10 μM acetazolamide. 20 μL of hCAII solution was added to all wells except no enzyme control well, in which case 20 μL hCAII assay buffer was added instead. Plates were incubated for 11 minutes at room temperature before addition of 4-NPA solution to all wells. Absorbance at 348 nm was read every 10 seconds of 11 minute total run time. Three technical replicates were assessed per plate. Controls: hCAII+4-NPA (DMSO added in place of compound of interest, DMSO control), 4-NPA background (no enzyme, DMSO added in place of compound of interest, 4-NPA control), hCAII+4-NPA+acetazolamide (acetazolamide added in place of compound of interest, known hCAII inhibitor).

Percent inhibition calculated as: 100−[100×[(Ab_{Saverage, compound of interest}−Ab_{Saverage, 4-NPA})/(Ab_{Saverage, DMSO}−Ab_{Saverage, 4-NPA})]].

Compound Aggregation Assay. Stock solutions of DHEA (5 mM in DMSO) and compound of interest (5 mM in DMSO) were prepared. For each 60 μL reaction, to a 1.5 mL Eppendorf tube was added 26.7 μL 1× assay buffer supplemented with 0.2 mg/mL bovine serum albumin (BSA) and 1.2 μL compound of interest or DMSO. BSA-containing 1× assay buffer and compound of interest were incubated at room temperature for 5 minutes before addition of 1.2 μL of 5 mM DHEA, and 0.9 μL 6.6 mM PAPS. SULT2B1b was diluted to 1.5 μg/μL in protein storage buffer then down to 0.8 μg/μL in 1× activity assay buffer. 30 μL of 0.8 μg/μL SULT2B1b was added to each reaction. Reactions were incubated for 2 hours at 37° C. with shaking at 250 rpm. To quench, 30 μL reaction mix was added to 30 μL ice cold 0.5% HCl in MeCN, then centrifuged at 13,000 xg at room temperature for 3 min. 50 μL supernatant was transferred to an empty Eppendorf tube and submitted for LC-MS/MS quantification (UIUC Metabolomics Center). For samples run without BSA, an identical protocol was followed using 26.7 μL 1× assay buffer that had not been supplemented with BSA. Controls: Reaction mix made with DHEA, DMSO in place of compound of interest (DHEA control).

Percent inhibition was calculated as: 100−[100×(DHEA-sulfate produced_{compound of interest}/DHEA sulfate produced_{DHEA})].

Western Blot Analysis. Cells were lysed using RIPA buffer containing phosphatase (BioVision) and protease inhibitor cocktail (Calbiochem). Protein concentrations were determined using the BCA assay (Pierce). Lysates containing 15 μg of protein were loaded onto 4-20% gradient gels (BioRad), and SDS-PAGE was run. Proteins were then transferred onto membrane (PVDF Millipore) for Western Blot analysis. Blots were blocked with BSA solution (2 g in 40 mL TBST) for one hour followed by primary antibody addition and incubation overnight (using manufacturer's recommended dilutions). Following overnight incubation, blots were washed with TBST, and incubated with HRP-linked secondary antibody for 1 hour in TBST. Blots were washed, then imaged with ChemiDoc after incubation with SuperSignal West Pico Solution following manufacturer's procedures.

Cell Culture. MCF-7 and HepG2 were grown in Eagle's minimum essential media supplemented with 10% fetal bovine serum (Benchmark, Gemini) and 1% penicillin/streptomycin. T47D and HCT-116 was grown in RPMI 1640 supplemented with 10% fetal bovine serum and 1% penicillin/streptomycin. MDA-MB-468 and BT-20 were grown in Dulbecco's modified Eagle's medium supplemented with 10% fetal bovine serum and 1% penicillin/streptomycin. Mouse embryonic fibroblasts were grown in Dulbecco's modified Eagle's medium supplemented with 10% fetal bovine serum, 1% penicillin/streptomycin, and an additional 1% non-essential amino acids. MYS and MDG were grown in Dulbecco's modified Eagle's medium supplemented with 5% fetal bovine serum and 1% penicillin/streptomycin. Phenol red was added to all media as a pH indicator. All cells were cultured at 37° C. in a 5% CO₂ environment. Media were prepared by the University of Illinois School of Chemical Sciences Cell Media Facility.

General Whole Cell Cholesterol Sulfate Quantification. 60×15 mm tissue culture dishes were seeded with MCF-7 at 2×10⁶ cells/dish or T47D at 1×10⁶ cells per dish, 5 mL total media volume, and allowed to adhere overnight. Compounds of interest were dissolved in DMSO. Cells were treated with 25 μL 1.25 mM compound of interest stock (6.25 μM final compound concentration, 0.5% final DMSO concentration) or DMSO for 72 hours. To harvest, media was aspirated, and cells were washed once with PBS before detaching with trypsin. Trypsin was quenched with media. Cells were centrifuged at 300 xg for 3 minutes and supernatant was discarded. Cells were resuspended in media and counted using a Countess 3 automated cell counter (Thermo Fisher Scientific). 1.5×10⁶ cells were collected, washed twice with 1 mL PBS, then pelleted and stored at −80° C. until lysis. Cell pellets were resuspended in 200 μL cold 70:30 methanol:water and placed on ice. Cell suspension was sonicated on ice (30%, 10 s on, 20 s off, 1 min total) and continued to be incubated on ice for 30 minutes. Lysate was clarified by centrifugation at 14,100 xg at 4° C. for 30 minutes. 100 μL of supernatant was transferred to an empty Eppendorf tube and submitted for LC-MS/MS quantification (UIUC Metabolomics Center), along with 5 mM cholesterol sulfate sodium salt standard.

Time Course Whole Cell Cholesterol Sulfate Quantification. 60×15 mm tissue culture dishes were seeded with MCF-7 at 2×10⁶ cells/dish per dish, 5 mL total media volume, and allowed to adhere overnight. Compounds of interest were dissolved in DMSO. Cells were treated with 25 μL 1.25 mM compound of interest stock (6.25 μM final compound concentration, 0.5% final DMSO concentration) or DMSO for 0 (harvested immediately), 4, 8, 24, 48, or 72 hours. To harvest, media was aspirated, and cells were washed once with PBS before detaching with trypsin. Trypsin was quenched with media. Cells were centrifuged at 300 xg for 3 minutes and supernatant was discarded. Cells were resuspended in media and counted using a Countess 3 automated cell counter (Thermo Fisher Scientific). 1.5×10⁶ cells were collected, washed twice with 1 mL PBS, then pelleted and stored at −80° C. until lysis. Cell pellets were resuspended in 200 μL cold 70:30 methanol:water and placed on ice. Cell suspension was sonicated on ice (30%, 10 s on, 20 s off, 1 min total) and continued to be incubated on ice for 30 minutes. Lysate was clarified by centrifugation at 14,100 xg at 4° C. for 30 minutes. 100 μL of supernatant was transferred to an empty Eppendorf tube and submitted for LC-MS/MS quantification (UIUC Metabolomics Center), along with 5 mM cholesterol sulfate sodium salt standard.

Whole Cell Cholesterol Sulfate Quantification with Compound Removal. 60×15 mm tissue culture dishes were seeded with MCF-7 at 2×10⁶ cells/dish, 5 mL total media volume, and allowed to adhere overnight. Compounds of interest were dissolved in DMSO. Cells were treated with 25 μL 1.25 mM compound of interest stock (6.25 μM final compound concentration, 0.5% final DMSO concentration) for 0 hours (harvested immediately) or 24 hours. Media was aspirated and replaced with 5 mL fresh media, then cells were either harvested (24 hour time point) or further incubated. To harvest, media was aspirated, and cells were washed once with PBS before detaching with trypsin. Trypsin was quenched with media. Cells were centrifuged at 300 xg for 3 minutes and supernatant was discarded. Cells were resuspended in media and counted using a Countess 3 automated cell counter (Thermo Fisher Scientific). 1.5×10⁶ cells were collected, washed twice with 1 mL PBS, then pelleted and stored at −80° C. until lysis. Cell pellets were resuspended in 200 μL cold 70:30 methanol:water and placed on ice. Cell suspension was sonicated on ice (30%, 10 s on, 20 s off, 1 min total) and continued to be incubated on ice for 30 minutes. Lysate was clarified by centrifugation at 14,100 xg at 4° C. for 30 minutes. 100 μL of supernatant was transferred to an empty Eppendorf tube and submitted for LC-MS/MS quantification (UIUC Metabolomics Center), along with 5 mM cholesterol sulfate sodium salt standard.

Alamar Blue Fluorescence for Cellular Activity (IC₅₀). Cells were seeded per well in a 96-well plate (5000 for MCF-7, 4000 for T47D, 7000 for mouse embryonic fibroblasts) and allowed to adhere before DMSO solutions of compounds were added to each well. Final concentration of DMSO in each well is 1%, final volume: 100 μL. At the end of 72 hours, Alamar Blue solution was added (10 μL of 1 mg resazurin per 10 mL PBS). After 2-4 hours incubation, fluorescence (λ_{excit.}=555 nm, λ_{emission.}=585 nm) was measured. The fluorescence of each well was read with a SpectraMax M3 plate reader (Molecular Devices). Percent dead was determined by comparison to a 100% dead control: 100 μM Raptinal treated cells.

Whole Cell Cholesterol Assay. Assay was run according to manufacturer's instructions. Briefly, 5000 MCF-7 cells were plated in 99 μL media in a 96-well plate and allowed to adhere overnight before DMSO solutions of compounds were added to each well. Final concentration of DMSO in each well is 1%, final volume: 100 μL. At the end of 72 hours, media was aspirated, and cells were washed twice with 100 μL PBS. 50 μL of cholesterol lysis solution was added and cells were incubated for 30 minutes at 37° C. 50 μL of sample was then transferred to a 96-well white walled assay plate. 50 μL of cholesterol detection reagent was added to all wells and incubated at room temperature for 1 hour. Luminescence was read using a SpectraMax M3 plate reader (Molecular Devices). Cholesterol levels were determined by use of a cholesterol standard curve.

IACUC Guidelines and Protocol Numbers. All mouse model work at UIUC was conducted in accordance with UIUC IACUC guidelines and approved protocols. The following approved IACUC protocols were used for the work described here: 20142.

Tolerability Studies. Three female C57/BL6 mice were administered single IP doses (formulated in 50% DMSO/50% Peg400) of compound (2). Compound was formulated at a concentration of 20-40 mg/mL depending on dose. Mice were injected with 100 μL formulated compound. Mouse weight and lethality were tracked as indicators of compound tolerability. Tolerability of vehicle had been previously assessed.

Pharmacokinetic Analysis. Female C57/BL6 mice were administered single IP doses (200 mg/kg in 50% DMSO/50% Peg400) of compound (2) and then sacrificed in cohorts of 3 at predetermined time points (0, 20, 30, 60, 120, and 240 minutes. Whole blood was collected, centrifuged, and EDTA serum separated for quantification by IPLC methods (UIUC Metabolomics Center, Urbana, IL).

Cholesterol Sulfate Levels in MDG Tumor Tissue. 1.5 or 3 million MDG cells suspended in 114atrigel were injected into the mammary fat pad of a female athymic nude mouse. Tumors were allowed to grow in the mice for 40 days following the graft, at which point tumors were harvested, flash frozen, and stored at −80° C. until further processing. Tumors were thawed on ice for 10 minutes before addition of 1 mL of cold methanol. A tissue homogenizer was used to emulsify tumor tissue, and the soluble portion was obtained by centrifugation at 2000 rpm at 4° C. for 10 minutes to separate the coarse particles, followed by transfer to an empty Eppendorf tube and centrifugation at 14,100 xg at 4° C. for 30 minutes. 600 μL of supernatant was transferred to an empty Eppendorf tube and submitted for LC-MS/MS quantification (UIUC Metabolomics Center), along with 5 mM cholesterol sulfate sodium salt standard.

General LC-MS/MS Quantification Method. Both DHEAS and CS samples were analyzed by 6500 QTRAP (Sciex, Redwood City, CA) liquid chromatography-tandem mass spectrometry. Subsequently, samples were injected (5 μL) into the Agilent 1260 Infinity II system, and equipped with an Agilent Zorbax SB-C3 column (2.1×50 mm, 5 μm) with mobile phase A (0.1% formic acid in water) and mobile phase B (0.1% formic acid in acetonitrile). The flow rate was 0.3 mL/min. Mass spectra were acquired under negative electrospray ionization with the ion spray voltage at −4500 V. The source temperature was 400° C. The curtain gas, ion source gas 1, and ion source gas 2 were 35, 50, and 65 pounds/square inch, respectively. Cholesterol-sulfate and DHEA-sulfate were detected by multiple reaction monitoring (MRM) at m/z 465→97.0 and 367.0→97.0 respectively. Software Analyst 1.7.1 (Agilent) was used for data acquisition and analysis.

### Example 2. Compound Screening

Testing of compound IB:10:D (1) in non-specific inhibition assays. Two assays were performed to test the inhibition specificity of IB:10:D. The first is a well-known assay utilizing human carbonic anhydrase II. No inhibition of hCAII is observed up to 200 μM of IB:10:D. The second is an assay developed to test the aggregation capacity of this class of compounds. In the presence of a decoy protein (Bovine serum albumin, BSA), no change in the inhibition of SULT2B1b is seen, suggesting this class of compounds does not exert its effect through compound aggregation.

Whole cell cytotoxicity-MCF-7. Cell cytotoxicity was assessed to ensure compound concentrations used the whole cell assays would not kill the cells. Alamar blue cell cytotoxicity assay: 5000 cells were seeded per well in a 96-well plate and allowed to adhere before DMSO solutions of compounds were added to each well. Final concentration of DMSO in each well is 1%, with a final volume of 100 μl. At the end of 72 hours Alamar blue solution was added [10 μl of 1-mg resazurin per 10 ml of phosphate-buffered saline (PBS)]. After 2 to 4 hours of incubation, fluorescence (λexcitation=555 nm, λemission=585 nm) was measured. Percent dead was determined by comparison to a 100% dead control: 100 μM Raptinal-treated cells (FIG. 6).

Using the whole cell assay described above, intracellular cholesterol sulfate levels were assessed in MCF-7 cells at 6.25 μM compound (3) and compound (6). A significant decrease in cholesterol sulfate levels was achieved with compound (3) treatment, but not seen with compound (6) treatment. This suggests that intracellular cholesterol sulfate level decreases are specific to compound (3) and not an artifact of this particular scaffold (FIG. 7).

Mouse studies with compound (3). Maximum tolerated dose of compound (3) was tested in mice at 100, 125, or 200 mg/kg, dosed intraperitoneally. No signs of toxicity were observed (toxicity defined as: weight loss>10% after 24 hours). Pharmacokinetic analysis was performed at 200 mg/kg dosed intraperitoneally. Compound (3) shows a favorable pharmacokinetic profile with compound concentrations reaching ˜50 μM in the serum (FIG. 10).

Maximum tolerated dose mouse studies: Compound (3) was formulated in 50% DMSO, 50% Peg400 at 20 mg/mL (for 100 mg/kg injection), 25 mg/mL (for 125 mg/kg injection), or 40 mg/mL (for 200 mg/kg injection). Compound was injected intraperitoneally into female C57BL/6 mice, and the mice were monitored for signs of toxicity.

Pharmacokinetic mouse studies: Compound (3) was formulated in 50% DMSO, 50% Peg400 at 40 mg/mL (for 200 mg/kg injection), Compound was injected intraperitoneally into female C57BL/6 mice (3 per time point). Blood from the mice were harvested at each time point. Serum was separated and concentration in the serum was quantified by LC-MS/MS.

### Example 3. Chemical Synthetic Methods

Materials and Methods. Reagents were purchased from commercial sources and used without further purification. All solvents used were anhydrous, resulting from being passed through activated alumina columns utilizing a PureSolv MD-5 solvent purification system. Compounds utilized for in vivo experiments were dried via overnight lyophilization from neat acetonitrile. ¹H NMR and ¹³C NMR experiments were conducted on a Bruker Avance III HD 500 MHz NMR with a CryoProbe. Spectra obtained in CDCl₃ were referenced for 7.26 ppm and 77.16 ppm for ¹H and ¹³C NMR spectra respectively. Spectra obtained in DMSO were referenced for 2.50 ppm and 39.52 for ¹H and ¹³C NMR spectra respectively. All NMR chemical shifts are reported in ppm (δ), coupling constants (J, Hz), and peaks reported as: s=singlet, d=doublet, t=triplet, q=quartet, m=multiplet. ¹³C and ¹⁹F peak multiplicities are all singlets unless otherwise noted with the same designation used for ¹H NMR. High resolution mass spectra (HRMS) were obtained at the UIUC SCS Mass Spectrometry Laboratory utilizing electrospray ionization (ESI). Final compound purity are reported via LC-MS analysis at k=254 nm.

### General Preparation of Enamide Compounds.

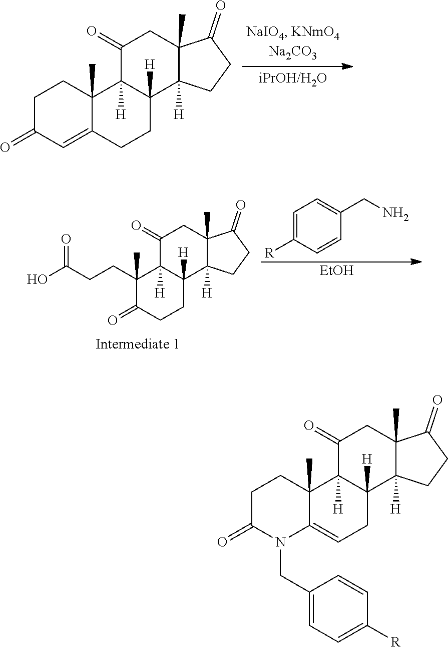

General Method A. Procedure adapted from Nat. Chem. 2013, 5, 195-202.

Adrenosterone (1 g, 3.32 mmol) was dissolved in isopropanol (20 mL) and sodium carbonate (660 mg, 6.22 mmol) dissolved in water (3 mL) was added to the resulting solution. The reaction mixture was heated to reflux. A solution of sodium periodate (5.2 g, 24.3 mmol) and catalytic potassium permanganate (0.106 mg, 0.671 mmol) in water (24 mL) was preheated at 75° C. and added to the reaction mixture dropwise using a slow addition funnel over a 30 minute period. The slow addition funnel was then removed, and a reflux condenser was placed on the reaction flask. The reaction was allowed to stir for an additional 2.5 hours before being cooled to room temperature. The reaction was filtered, and the remaining solids were washed with water. The isopropanol was then removed under reduced pressure and the remaining aqueous solution was acidified with concentrated hydrochloric acid to pH 2. This aqueous solution was extracted with dichloromethane (3×). The organic layers were collected, dried using sodium sulfate and concentrated under reduced pressure. The compound was purified by flash column chromatography (1% to 3% MeOH in DCM).

Final compounds, adapted from above reference: Intermediate 1 (150 mg, 0.465 mmol) was dissolved in ethanol (3 mL) in a sealed tube and various benzylamines (2.5 eq.) were added the solution. The tube was sealed and heated to 125° C. for 16 hours before being cooled to room temperature. A 5% solution of aqueous hydrochloric acid solution was added to the reaction vessel and allowed to stir for 5 minutes before being transferred to a separatory funnel where dichloromethane was used to extract the mixture (3×). The organic layers were combined, dried with sodium sulfate and concentrated under reduced pressure. The product was purified by flash column chromatography using 30% EtOAc to 50% EtOAc in hexanes.

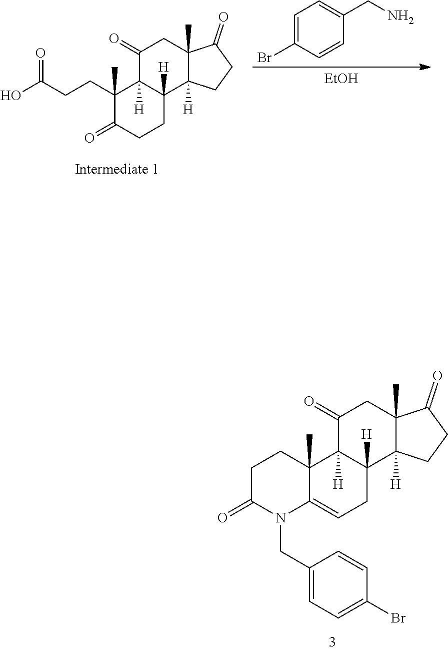

Synthesis of compound (3). Intermediate 1 (172 mg, 0.537 mmol) was dissolved in ethanol (3 mL) in a sealed tube and 4-bromobenzylamine (0.17 mL, 1.342 mmol) was added to the solution. The tube was sealed and heated to 125° C. for 16 hours before being cooled to room temperature. A 5% solution of aqueous hydrochloric acid solution was added to the reaction vessel and allowed to stir for 5 minutes before being transferred to a separatory funnel where dichloromethane was used to extract the mixture (3×). The organic layers were combined, dried with sodium sulfate and concentrated under reduced pressure. The product was purified by flash column chromatography using 30% EtOAc to 50% EtOAc in hexanes, obtaining 138 mg of a white foam (55% yield). ¹H NMR (500 MHz, CDCl₃): δ 7.27 (d, J=8.4 Hz, 2H), 6.88 (d, J=8.1 Hz, 2H), 4.93 (d, J=15.9 Hz, OH), 4.84-4.76 (m, OH), 4.51 (d, J=15.9 Hz, 1H), 2.65-2.35 (m, 5H), 2.25-2.07 (m, 3H), 2.00-1.67 (m, 6H), 1.52 (tt, J=12.5, 9.3 Hz, 1H), 1.37-1.26 (m, 1H), 1.08 (s, 3H), 0.71 (s, 3H). ¹³C NMR (126 MHz, CDCl₃) δ 217.21, 207.80, 169.10, 143.67, 136.90, 131.79, 128.54, 120.79, 103.87, 59.78, 50.43, 50.06, 49.92, 47.53, 36.22, 35.93, 32.07, 30.64, 30.32, 28.94, 21.83, 18.09, 14.93.

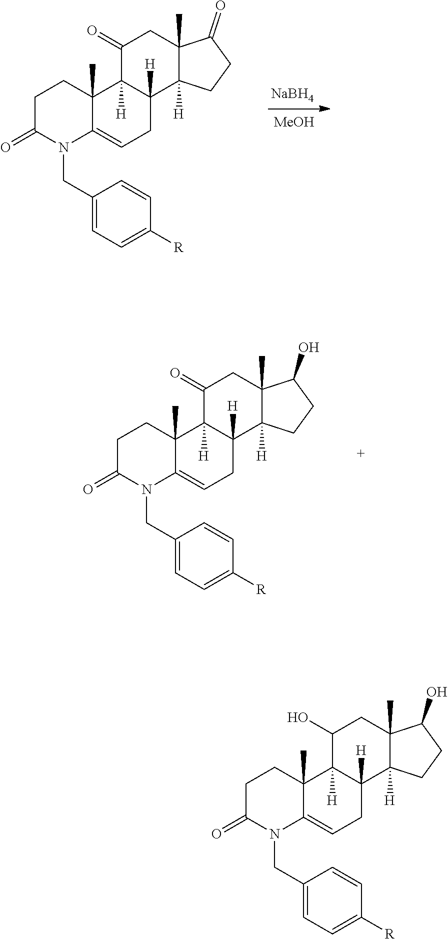

General preparation of C-17 and C-11 reduced ketones. Substituted enamide starting material (1 eq.) was dissolved in methanol (20 mL) in a round bottom flask and stirred at 0° C. for 10 min. NaBH₄ (1.1 eq) was added, and reaction was removed from ice bath and stirred at room temperature for 1 hour. Reaction was quenched by addition of water, then evaporated under reduced pressure before being transferred to a separatory funnel where ethyl acetate was used to extract the mixture (3×). The organic layers were combined, dried with sodium sulfate and concentrated under reduced pressure. The product was purified by flash column chromatography using 70% EtOAc in hexanes to 100% EtOAc.

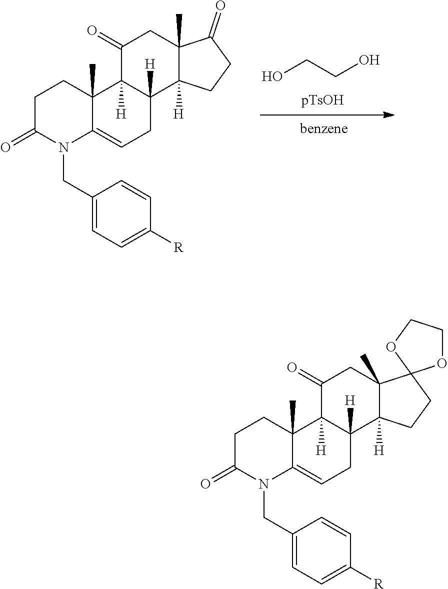

General preparation of C-17 ketals. Substituted enamide starting material (1 eq.) was dissolved in benzene (40 mL) in a round bottom flask. p-Toluenesulfonic acid (0.3 eq.) was added to the reaction followed by ethylene glycol (50 eq.). A Dean-Stark trap was fitted to the reaction flask and the reaction was heated at reflux for 16 hours. At this time, the reaction was cooled to room temperature, diluted with ethyl acetate and transferred to a separatory funnel where the organic layer was washed with saturated sodium bicarbonate (1×) and DI H₂O (1×). The organic layer was dried with sodium sulfate and concentrated under reduced pressure. The product was purified by flash column chromatography using 30% to 70% EtOAc in hexanes.

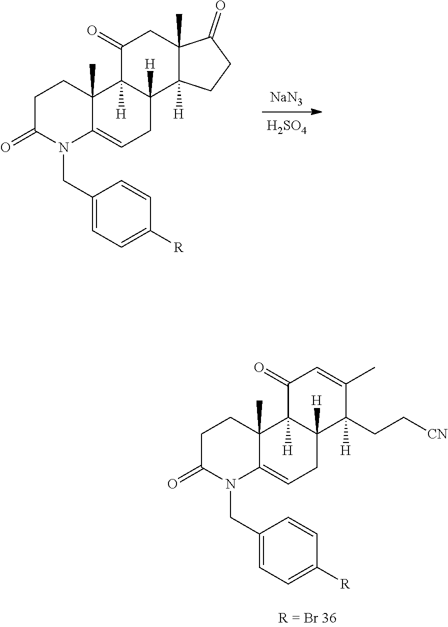

Preparation of compound 36. Prepared according to Nat. Chem. 2013, 5, 195-202. Substituted enamide starting material (1 eq.) was dissolved in concentrated sulfuric acid (2 mL) at room temperature before cooling to 0° C. Sodium azide (2 eq.) was then added to the reaction slowly and the resulting reaction mixture was allowed to stir for 1 hour at 0° C. After this time, ice was added to quench the reaction and stirring continued for an additional 3 minutes before being transferred to a separatory funnel and partitioned between brine and dichloromethane. Dichloromethane was used to extract the desired Schmidt products (3×). The organic layers were combined, dried with sodium sulfate and concentrated under reduced pressure. The product was purified via column chromatography using 50% EtOAc to 70% EtOAc in hexanes.

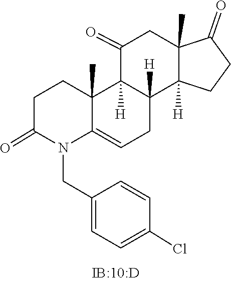

(4aR,4bS,6aS,9aS,9bS)-1-(4-chlorobenzyl)-4a,6a-dimethyl-3,4,4a,6,6a,8,9,9a,9b,10-decahydro-1H-indeno[5,4-f]quinoline-2,5,7(4bH)-trione (IB:10:D, (1)). Synthesized via general method A. Purified by silica gel chromatography 30% EtOAc/Hexanes to 50% EtOAc/Hexanes. Product obtained as an off-white solid in 50% yield. ¹H NMR: (500 MHz, CDCl₃) δ: 7.26 (d, J=8.4 Hz, 2H), 7.08 (d, J=8.4 Hz, 2H), 5.10 (d, J=15.9 Hz, 1H), 4.95 (dd, 1H), 4.67 (d, J=15.8 Hz, 1H), 2.81-2.68 (m, 2H), 2.67-2.51 (m, 3H), 2.40-2.22 (m, 3H), 2.15-2.07 (m, 1H), 2.04-1.84 (m, 4H), 1.74-1.61 (m, 1H), 1.53-1.42 (m, 1H), 1.23 (s, 3H), 0.85 (s, 3H). ¹³C NMR (126 MHz, CDCl₃) δ 217.21, 207.80, 169.11, 143.67, 136.35, 132.73, 128.84, 128.16, 103.89, 59.77, 50.42, 50.05, 49.91, 47.49, 36.22, 35.93, 32.07, 30.64, 30.32, 28.94, 21.82, 18.08, 14.92. HRMS (ESI): m/z calc. for C₂₅H₂₈ClNO₃ [M+H]⁺ 426.1758, found: 426.1832. LC-MS Purity (λ: 254 nm): 96.1%.

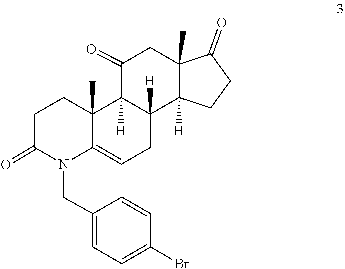

(4aR,4bS,6aS,9aS,9bS)-1-(4-bromobenzyl)-4a,6a-dimethyl-3,4,4a,6,6a,8,9,9a,9b,10-decahydro-1H-indeno[5,4-f]quinoline-2,5,7(4bH)-trione (compound 3). Synthesized via general method A. Purified by silica gel chromatography 30% EtOAc/Hexanes to 50% EtOAc/Hexanes. Product obtained as an off-white solid in 45% yield. ¹H NMR: (500 MHz, CDCl₃) δ: 7.42 (d, J=8.4 Hz, 2H), 7.03 (d, J=8.1 Hz, 2H), 5.08 (d, J=15.9 Hz, 1H), 4.95 (dd, J=5.8, 2.0 Hz, 1H), 4.66 (d, J=15.9 Hz, 1H), 2.78-2.68 (m, 2H), 2.67-2.52 (m, 3H), 2.40-2.23 (m, 3H), 2.15-2.07 (m, 1H), 2.04-1.84 (m, 4H), 1.74-1.63 (m, 1H), 1.51-1.43 (m, 1H), 1.23 (s, 3H), 0.86 (s, 3H). ¹³C NMR: (126 MHz, CDCl₃) δ: 217.21, 207.80, 169.10, 143.67, 136.90, 131.79, 128.54, 120.79, 103.87, 59.78, 50.43, 50.06, 49.92, 47.53, 36.22, 35.93, 32.07, 30.64, 30.32, 28.94, 21.83, 18.09, 14.93. HRMS (ESI): m/z calc. for C₂₅H₂₈BrNO₃ [M+H]⁺ 470.1253, found: 470.1327. LC-MS Purity (λ: 254 nm): 97.3%.

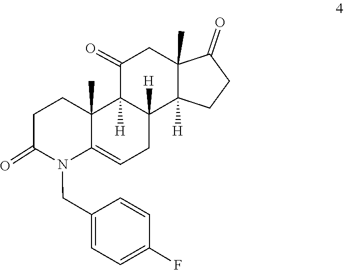

(4aR,4bS,6aS,9aS,9bS)-1-(4-fluorobenzyl)-4a,6a-dimethyl-3,4,4a,6,6a,8,9,9a,9b,10-decahydro-1H-indeno[5,4-f]quinoline-2,5,7(4bH)-trione (compound 4). Synthesized via general method A. Purified by silica gel chromatography 30% EtOAc/Hexanes to 50% EtOAc/Hexanes. Product obtained as an off-white solid in 56% yield. ¹H NMR (500 MHz, CDCl₃) δ: 7.14-7.10 (m, 2H), 7.02-6.95 (m, 2H), 5.11 (d, J=15.7 Hz, 1H), 4.99 (dd, J=5.7, 2.0 Hz, 1H), 4.67 (d, J=15.7 Hz, 1H), 2.80-2.68 (m, 2H), 2.67-2.52 (m, 3H), 2.41-2.23 (m, 3H), 2.14-2.07 (m, 1H), 2.04-1.92 (m, 3H), 1.91-1.84 (m, 1H), 1.73-1.63 (m, 1H), 1.52-1.42 (m, 1H), 1.22 (s, 3H), 0.85 (s, 3H). ¹³C NMR (126 MHz, CDCl₃) δ: 217.21, 207.81, 169.08, 161.92 (d, J=244.87 Hz), 143.72, 133.50 (d, J=3.06 Hz), 128.36 (d, J=8.11 Hz), 115.53 (d, J=21.56 Hz), 103.87, 59.80, 50.42, 50.05, 49.92, 47.46, 36.22, 35.94, 32.08, 30.67, 30.34, 28.97, 21.83, 18.07, 14.92. HRMS (ESI): m/z calc. for C₂₅H₂₈FNO₃ [M+H]⁺ 410.2053, found: 410.2128. LC-MS Purity (λ: 254 nm): 98.0%.

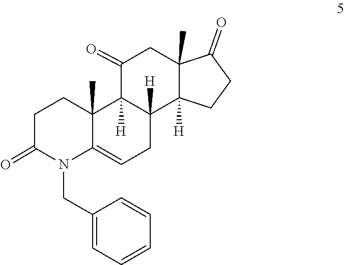

(4aR,4bS,6aS,9aS,9bS)-1-benzyl-4a,6a-dimethyl-3,4,4a,6,6a,8,9,9a,9b,10-decahydro-1H-indeno[5,4-f]quinoline-2,5,7(4bH)-trione (compound 5). Synthesized via general method A. Purified by silica gel chromatography 30% EtOAc/Hexanes to 50% EtOAc/Hexanes. Product obtained as an off-white solid in 53% yield. ¹H NMR (500 MHz, CDCl₃) δ: 7.32-7.28 (m, 2H), 7.24-7.20 (m, 1H), 7.16-7.13 (m, 2H), 5.22 (d, J=15.8 Hz, 1H), 5.00 (dd, J=5.7, 2.2 Hz, 1H), 4.64 (d, J=15.8 Hz, 1H), 2.79-2.69 (m, 2H), 2.68-2.52 (m, 3H), 2.40-2.23 (m, 3H), 2.14-2.06 (m, 1H), 2.05-1.84 (m, 4H), 1.72-1.61 (m, 1H), 1.53-1.45 (m, 1H), 1.25 (s, 3H), 0.85 (s, 3H). ¹³C NMR (126 MHz, CDCl₃) δ: 217.27, 207.88, 169.07, 143.82, 137.83, 128.68, 126.95, 126.61, 103.83, 59.83, 50.43, 50.06, 49.94, 48.26, 36.23, 35.94, 32.09, 30.72, 30.36, 28.99, 21.83, 18.13, 14.91. HRMS (ESI): m/z calc. for C₂₅H₂₉NO₃ [M+H]⁺ 392.2147, found: 392.2221. LC-MS Purity (λ: 254 nm): 96.2%.

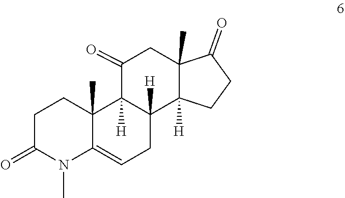

(4aR,4bS,6aS,9aS,9bS)-1,4a,6a-trimethyl-3,4,4a,6,6a,8,9,9a,9b,10-decahydro-1H-indeno[5,4-f]quinoline-2,5,7(4bH)-trione (compound 6). Synthesized via general method A with minor modifications. 33% Methylamine in EtOH (6.5 equivalents) used in place of neat amine. Purified by silica gel chromatography 70% EtOAc/Hexanes to 100% EtOAc. Product obtained as an off-white solid in 44% yield. ¹H NMR (500 MHz, CDCl₃) δ: 5.05 (dd, J=5.7, 1.9 Hz, 1H), 3.13 (s, 3H), 2.72-2.65 (m, 1H), 2.64-2.46 (m, 5H), 2.36-2.26 (m, 2H), 2.21-2.13 (m, 1H), 2.10-1.99 (m, 3H), 1.98-1.89 (m, 1H), 1.77-1.66 (m, 1H), 1.44-1.35 (m, 1H), 1.25 (s, 3H), 0.88 (d, J=0.9 Hz, 3H). ¹³C NMR (126 MHz, CDCl₃) δ: 217.31, 207.86, 168.88, 145.29, 102.78, 59.95, 50.43, 50.07, 49.93, 36.25, 35.89, 32.23, 31.82, 30.98, 30.42, 28.93, 21.90, 18.38, 14.93. HRMS (ESI): m/z calc. for C₁₉H₂₅NO₃ [M+H]⁺ 316.1834, found: 316.1902. LC-MS Purity (λ: 254 nm): >99%.

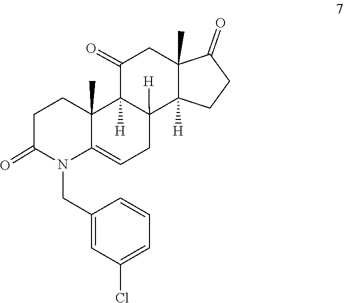

(4aR,4bS,6aS,9aS,9bS)-1-(3-chlorobenzyl)-4a,6a-imethy-3,4,4a,6,6a,8,9,9a,9b,10-decahydro-1H-indeno[5,4-f]quinoline-2,5,7(4bH)-trione (compound 7). Synthesized via general method A. Purified by silica gel chromatography 30% EtOAc/Hexanes to 50% EtOAc/Hexanes. Product obtained as an off-white solid in 28% yield. ¹H NMR (500 MHz, CDCl₃) δ: 7.25-7.19 (m, 2H), 7.12 (t, J=1.8 Hz, 1H), 7.05-7.02 (m, 1H), 5.19 (d, J=16.0 Hz, 1H), 4.95 (dd, J=5.7, 2.1 Hz, 1H), 4.60 (d, J=16.0 Hz, 1H), 2.78-2.62 (m, 3H), 2.60-2.53 (m, 2H), 2.41-2.34 (m, 1H), 2.34-2.24 (m, 2H), 2.15-2.08 (m, 1H), 2.06-1.92 (m, 3H), 1.92-1.85 (m, 1H), 1.73-1.63 (m, 1H), 1.52-1.45 (m, 1H), 1.26 (s, 3H), 0.86 (s, 3H). ¹³C NMR (126 MHz, CDCl₃) δ: 217.23, 207.82, 169.12, 143.85, 140.00, 134.65, 130.01, 127.26, 126.73, 124.80, 103.87, 59.76, 50.43, 50.07, 49.93, 47.85, 36.23, 35.95, 32.09, 30.62, 30.34, 28.93, 21.84, 18.13, 14.93. HRMS (ESI): m/z calc. for C₂₅H₂₈ClNO₃ [M+H]⁺ 426.1758, found: 426.1826. LC-MS Purity (λ: 254 nm): 98.3%.

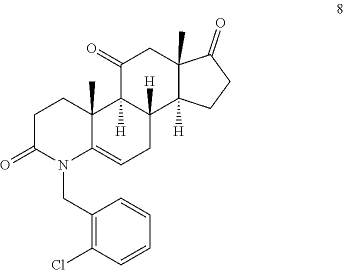

(4aR,4bS,6aS,9aS,9bS)-1-(2-chlorobenzyl)-4a,6a-dimethyl-3,4,4a,6,6a,8,9,9a,9b,10-decahydro-1H-indeno[5,4-f]quinoline-2,5,7(4bH)-trione (compound 8). Synthesized via general method A. Purified by silica gel chromatography 30% EtOAc/Hexanes to 50% EtOAc/Hexanes. Product obtained as an off-white solid in 40% yield. ¹H NMR (500 MHz, CDCl₃) δ: 7.38-7.32 (m, 1H), 7.21-7.14 (m, 2H), 6.97-6.93 (m, 1H), 5.33 (d, J=16.8 Hz, 1H), 4.83 (dd, J=5.7, 2.4 Hz, 1H), 4.60 (d, J=16.8 Hz, 1H), 2.81-2.71 (m, 2H), 2.70-2.62 (m, 1H), 2.59-2.51 (m, 2H), 2.40-2.22 (m, 3H), 2.14-2.06 (m, 1H), 2.05-1.83 (m, 4H), 1.71-1.61 (m, 1H), 1.53-1.46 (m, 1H), 1.30 (s, 3H), 0.86 (d, J=0.9 Hz, 3H). ¹³C NMR (126 MHz, CDCl₃) δ: 217.25, 207.87, 169.10, 143.81, 134.63, 132.51, 129.61, 128.17, 127.12, 127.00, 103.68, 59.80, 50.44, 50.07, 49.94, 46.55, 36.23, 35.97, 32.12, 30.76, 30.35, 28.98, 21.84, 18.24, 14.93. HRMS (ESI): m/z calc. for C₂₅H₂₈ClNO₃ [M+H]⁺ 426.1758, found: 426.1830. LC-MS Purity (λ: 254 nm): 95.2%.

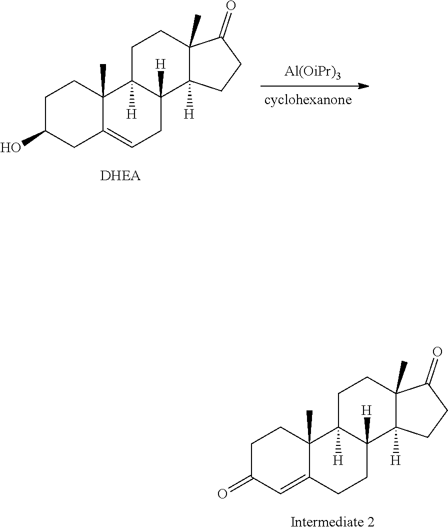

(8R,9S,10R,13S,14S)-10,13-dimethyl-1,6,7,8,9,10,11,12,13,14,15,16-dodecahydro-3H-cyclopenta[a]phenanthrene-3,17(2H)-dione (Intermediate 2). DHEIA (1.77 mmol, 1 eq.), cyclohexanone (3.6 mL, 20 eq.), and toluene (31 mL) were added to a round bottom flask and heated to reflux (115° C.) under N₂. After 15 minutes, Al(OiPr)₃ was added to the reaction. The reaction was heated to reflux again for 2 hours. After cooling to room temperature, reaction was transferred to a separatory funnel and washed with water, followed by 5M H₂SO₄, sat. NaHCO₃, and brine. The organic layer was dried with sodium sulfate, filtered over cotton, and concentrated by rotary evaporation. The product was purified by silica gel chromatography using 20% EtOAc/Hexanes to 50% EtOAc/Hexanes. Product obtained as an off-white solid in 70% yield. ¹H NMR (500 MHz, CDCl₃) δ: 5.75 (s, 1H), 2.51-2.30 (m, 5H), 2.15-2.02 (m, 2H), 2.01-1.95 (m, 2H), 1.87 (ddd, J=13.0, 4.2, 2.7 Hz, 1H), 1.78-1.66 (m, 3H), 1.63-1.54 (m, 1H), 1.51-1.41 (m, 1H), 1.34-1.25 (m, 2H), 1.21 (s, 3H), 1.17-1.07 (m, 1H), 1.04-0.95 (m, 1H), 0.92 (s, 3H). ¹³C NMR: (126 MHz, CDCl₃) δ 220.48, 199.42, 170.39, 124.31, 53.97, 51.00, 47.65, 38.78, 35.89, 35.85, 35.31, 34.06, 32.70, 31.43, 30.90, 21.89, 20.46, 17.52, 13.85. HRMS (ESI): m/z calc. for C₁₉H₂₆O₂ [M+H]⁺ 287.1933, found: 287.2003.

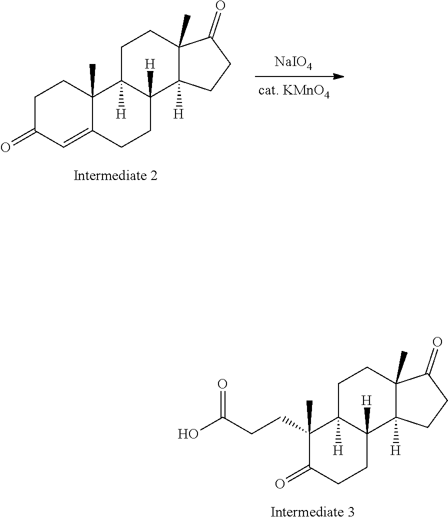

3-((3aS,5aS,6R,9aR,9bS)-3a,6-dimethyl-3,7-dioxododecahydro-1H-cyclopenta[a]naphthalen-6-yl)propanoic acid (Intermediate 3). Protocol was adapted from Nat. Chem. 2013, 5, 195-202. Intermediate 2 (2.25 mmol, 1 eq.), was dissolved in isopropanol and added to a 3-neck round bottom flask. Na₂CO₃ (4.5 mmol, 2 eq.) was dissolved in 2 mL DI water and added to the reaction, which was then equipped with a reflux condenser and an addition funnel before being heated to reflux for 15 minutes. In a separate 20 mL scintillation vial, 15 mL DI water was heated to 75° C. for 15 minutes. NaIO₄ (16.9 mmol, 7.5 eq) and KmnO₄ (0.240 mmol, 0.1 eq) were added to the heated water and stirred briefly to dissolve before being transferred to the addition funnel. The NaIO₄/KmnO₄ solution was added to the reaction over 30 minutes. The reaction was refluxed for 3 hours. After cooling to room temperature, solids were removed by vacuum filtration and washed with DI water. Remaining isopropanol was removed from the filtrate by rotary evaporation. Remaining liquid was transferred to a separatory funnel, acidified with 1M HCl, and extracted with dichloromethane (3×). Combined organic layers were washed with DI water (1×), then dried with sodium sulfate, filtered over cotton, and concentrated by rotary evaporation. The product was purified by silica gel chromatography using 3% MeOH/DCM to 5% MeOH/DCM. Product obtained as a white foam in 73% yield. ¹H NMR (500 MHz, DMSO) δ: 11.96 (s, 1H), 2.70-2.60 (m, 1H), 2.46-2.38 (m, 1H), 2.17-2.11 (m, 1H), 2.09-1.85 (m, 7H), 1.66 (dt, J=12.7, 3.3 Hz, 1H), 1.60-1.40 (m, 4H), 1.34-1.16 (m, 4H), 1.07 (s, 3H), 0.85 (s, 3H). ¹³C NMR (126 MHz, DMSO) δ: 219.31, 213.62, 174.66, 49.87, 49.81, 46.99, 46.88, 46.70, 37.32, 35.22, 33.54, 30.71, 29.37, 29.32, 28.91, 21.37, 20.78, 20.24, 20.21, 13.37, 13.29. HRMS (ESI): m/z calc. for C₁₈H₂₆O₄ [M+H]⁺ 307.1831, found: 307.1922.

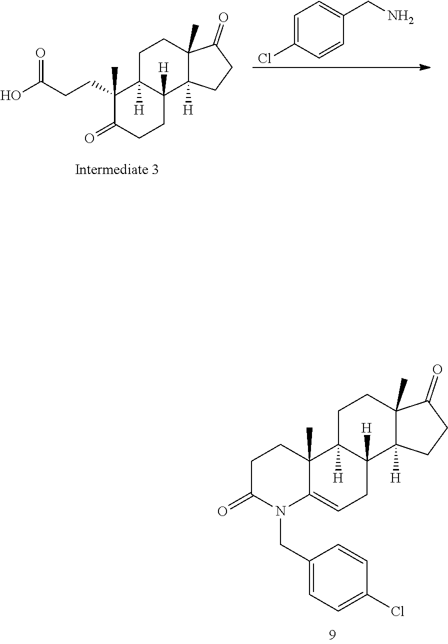

(4aR,4bS,6aS,9aS,9bR)-1-(4-chlorobenzyl)-4a,6a-dimethyl-3,4,4a,4b,5,6,6a,8,9,9a,9b,10-dodecahydro-1H-indeno[5,4-f]quinoline-2,7-dione (compound 9). Synthesized via general method A. Purified by silica gel chromatography 30% EtOAc/Hexanes to 50% EtOAc/Hexanes. Product obtained as an off-white foam in 28% yield. ¹H NMR (500 MHz, CDCl₃) δ: 7.26 (d, J=8.4 Hz, 2H), 7.09 (d, J=8.4 Hz, 2H), 5.09 (d, J=15.8 Hz, 1H), 4.97 (dd, J=5.7, 2.3 Hz, 1H), 4.71 (d, J=15.8 Hz, 1H), 2.72-2.58 (m, 2H), 2.47 (ddd, J=19.3, 9.0, 1.1 Hz, 1H), 2.23 (dt, J=16.6, 5.0 Hz, 1H), 2.14-2.04 (m, 1H), 2.01-1.91 (m, 2H), 1.90-1.85 (m, 1H), 1.77-1.63 (m, 3H), 1.60-1.43 (m, 3H), 1.35-1.24 (m, 2H), 1.18 (ddd, J=12.3, 10.6, 4.5 Hz, 1H), 1.06 (s, 3H), 0.89 (s, 3H). ¹³C NMR (126 MHz, CDCl₃) δ: 220.53, 168.74, 142.94, 136.47, 132.64, 128.79, 128.17, 105.15, 51.62, 48.97, 47.65, 47.11, 35.92, 35.76, 31.53, 31.35, 30.63, 29.63, 29.02, 21.89, 20.47, 18.76, 13.73. HRMS (ESI): m/z calc. for C₂₅H₃₀ClNO₂ [M+H]⁺ 412.1965, found: 412.2031. LC-MS Purity (λ: 254 nm): 98.2%.

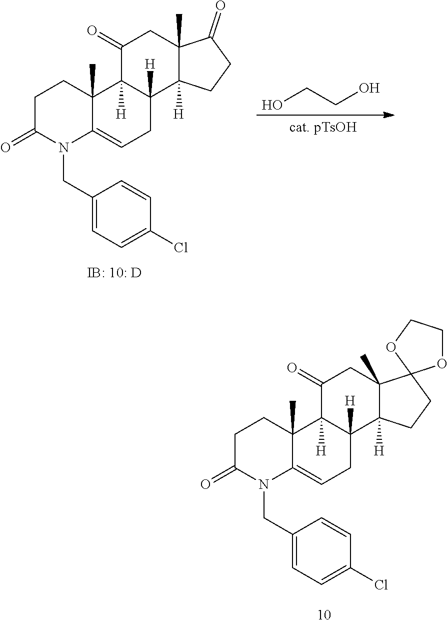

(4aR,4bS,6aS,9aS,9bS)-1-(4-chlorobenzyl)-4a,6a-dimethyl-1,3,4,4a,4b,6,6a,8,9,9a,9b,10-dodecahydrospiro[indeno[5,4-f]quinoline-7,2′-[1,3]dioxolane]-2,5-dione (compound 10). Substituted enamide starting material (0.269 mmol, 1 eq.) was dissolved in benzene (40 mL) in a round bottom flask. p-Toluenesulfonic acid (0.084 mmol, 0.3 eq.) was added to the reaction followed by ethylene glycol (50 eq.). A Dean-Stark trap was fitted to the reaction flask and the reaction was heated at reflux for 16 hours. At this time, the reaction was cooled to room temperature, diluted with EtOAc and transferred to a separatory funnel where the organic layer was washed with saturated NaHCO₃(1×) and DI water (1×). The organic layer was dried with sodium sulfate, filtered over cotton, and concentrated by rotary evaporation. The product was purified by silica gel chromatography using 30% EtOAc/Hexanes to 50% EtOAc/Hexanes. ¹H NMR (500 MHz, CDCl₃) δ: 7.26 (d, J=8.4 Hz, 2H), 7.08 (d, J=8.4 Hz, 2H), 5.11 (d, J=15.8 Hz, 1H), 4.92 (dd, J=5.8, 2.1 Hz, 1H), 4.63 (d, J=15.9 Hz, 1H), 3.97-3.88 (m, 2H), 3.83 (dd, J=9.5, 3.4 Hz, 2H), 2.76-2.67 (m, 2H), 2.65-2.58 (m, 2H), 2.28-2.19 (m, 1H), 2.15 (d, J=13.2 Hz, 1H), 2.09-1.97 (m, 3H), 1.96-1.78 (m, 4H), 1.50-1.32 (m, 2H), 1.21 (s, 3H), 0.82 (s, 3H). ¹³C NMR: (126 MHz, CDCl₃) δ 210.18, 169.23, 143.57, 136.48, 132.66, 128.81, 128.16, 117.87, 104.49, 65.55, 64.75, 59.15, 50.13, 48.97, 48.91, 47.56, 35.76, 34.42, 33.00, 30.82, 30.69, 28.99, 22.48, 17.99, 15.10. HRMS (ESI): m/z calc. for C₂₇H₃₂ClNO₄ [M+H]⁺ 470.2020, found: 470.2102. LC-MS Purity (λ: 254 nm): >99%.

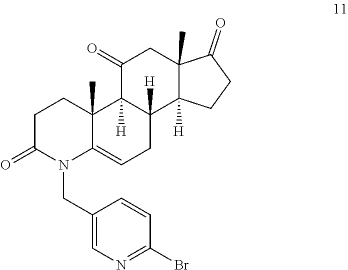

(4aR,4bS,6aS,9aS,9bS)-1-((6-bromopyridin-3-yl)methyl)-4a,6a-dimethyl-3,4,4a,6,6a,8,9,9a,9b,10-decahydro-1H-indeno[5,4-f]quinoline-2,5,7(4bH)-trione (compound 11). Synthesized via general method A. Purified by silica gel chromatography 30% EtOAc/Hexanes to 70% EtOAc/Hexanes. Product obtained as an off-white solid in 46% yield. ¹H NMR (500 MHz, CDCl₃) δ: 8.20 (s, 1H), 7.46-7.37 (m, 2H), 4.99-4.94 (m, 2H), 4.83 (d, J=15.9 Hz, 1H), 2.76-2.66 (m, 2H), 2.66-2.52 (m, 3H), 2.44-2.35 (m, 1H), 2.34-2.24 (m, 2H), 2.16-2.09 (m, 1H), 2.04-1.94 (m, 3H), 1.92-1.85 (m, 1H), 1.74-1.64 (m, 1H), 1.51-1.41 (m, 1H), 1.18 (s, 3H), 0.85 (s, 3H). ¹³C NMR (126 MHz, CDCl₃) δ: 217.11, 207.66, 169.22, 148.99, 143.41, 140.81, 137.82, 132.89, 128.27, 104.07, 59.70, 50.39, 50.02, 49.83, 44.61, 36.21, 35.98, 32.05, 30.52, 30.29, 28.88, 21.82, 17.96, 14.93. HRMS (ESI): m/z calc. for C₂₄H₂₇BrNO₃ [M+H]⁺ 471.1205, found: 471.1260. LC-MS Purity (λ: 254 nm): 96.0%.

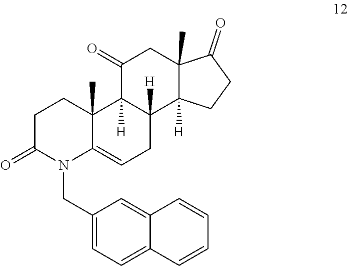

(4aR,4bS,6aS,9aS,9bS)-4a,6a-dimethyl-1-(naphthalen-2-ylmethyl)-3,4,4a,6,6a,8,9,9a,9b,10-decahydro-1H-indeno[5,4-f]quinoline-2,5,7(4bH)-trione (compound 12). Synthesized via general method A. Purified by silica gel chromatography 30% EtOAc/Hexanes to 50% EtOAc/Hexanes. Product obtained as an off-white solid in 64% yield. ¹H NMR (500 MHz, CDCl₃) δ: 7.84-7.76 (m, 3H), 7.57-7.56 (m, 1H), 7.50-7.42 (m, 2H), 7.32-7.28 (m, 1H), 5.29 (d, J=15.8 Hz, 1H), 5.07 (dd, J=5.8, 2.1 Hz, 1H), 4.91 (d, J=15.8 Hz, 1H), 2.84-2.66 (m, 3H), 2.57-2.50 (m, 2H), 2.36-2.21 (m, 3H), 2.11-2.00 (m, 2H), 2.00-1.82 (m, 3H), 1.68-1.60 (m, 1H), 1.55-1.49 (m, 1H), 1.26 (s, 3H), 0.83 (s, 3H). ¹³C NMR (126 MHz, CDCl₃) δ: 217.29, 207.89, 169.21, 143.61, 135.40, 133.53, 132.69, 128.51, 127.82, 127.80, 126.24, 125.77, 125.28, 125.10, 104.06, 59.82, 50.43, 50.04, 49.90, 48.16, 36.21, 35.98, 32.05, 30.73, 30.34, 29.05, 21.79, 18.14, 14.90. HRMS (ESI): m/z calc. for C₂₉H₃₁NO₃ [M+H]⁺ 442.2304, found: 442.2384. LC-MS Purity (k: 254 nm): 98.2%.

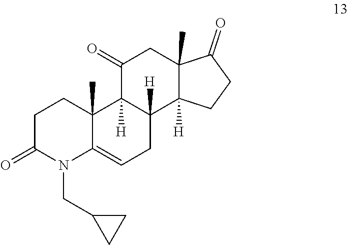

(4aR,4bS,6aS,9aS,9bS)-1-(cyclopropylmethyl)-4a,6a-dimethyl-3,4,4a, 6,6a, 8,9,9a, 9b,10-decahydro-1H-indeno[5,4-f]quinoline-2,5,7(4bH)-trione (compound 13). Synthesized via general method A. Purified by silica gel chromatography 30% EtOAc/Hexanes to 50% EtOAc/Hexanes. Product obtained as an off-white solid in 48% yield. ¹H NMR (500 MHz, CDCl₃) δ: 5.23 (dd, J=5.9, 1.9 Hz, 1H), 3.63 (dd, J=6.8, 2.2 Hz, 2H), 2.67-2.54 (m, 4H), 2.54-2.44 (m, 2H), 2.37-2.26 (m, 2H), 2.22-2.14 (m, 1H), 2.12-2.03 (m, 3H), 1.97-1.91 (m, 1H), 1.79-1.68 (m, 1H), 1.47-1.38 (m, 1H), 1.25 (s, 3H), 1.10-1.02 (m, 1H), 0.88 (s, 3H), 0.54-0.44 (m, 1H), 0.44-0.37 (m, 2H), 0.34-0.26 (m, 1H). ¹³C NMR (126 MHz, CDCl₃) δ: 217.34, 207.86, 168.92, 144.02, 103.63, 60.08, 50.42, 50.04, 49.92, 47.72, 36.27, 36.18, 32.28, 30.88, 30.55, 29.00, 21.92, 18.33, 14.95, 9.65, 4.34, 3.76. HRMS (ESI): m/z calc. for C₂₂H₂₉NO₃ [M+H]⁺ 356.2147, found: 356.2214. LC-MS Purity (λ: 254 nm): 98.1%.

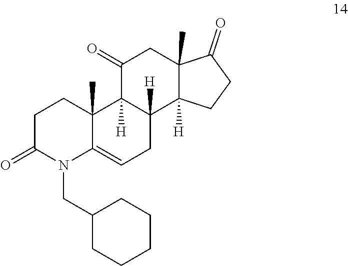

(4aR,4bS, 6aS, 9aS, 9bS)-1-(cyclohexylmethyl)-4a, 6a-dimethyl-3,4,4a, 6,6a, 8,9,9a, 9b,10-decahydro-1H-indeno[5,4-f]quinoline-2,5,7(4bH)-trione (compound 14). Synthesized via general method A. Purified by silica gel chromatography 30% EtOAc/Hexanes to 50% EtOAc/Hexanes. Product obtained as an off-white solid in 19% yield. ¹H NMR (500 MHz, CDCl₃) δ: 5.07 (dd, J=5.9, 1.9 Hz, 1H), 3.77 (dd, J=14.0, 8.3 Hz, 1H), 3.43 (dd, J=14.0, 5.5 Hz, 1H), 2.67-2.44 (m, 7H), 2.36-2.25 (m, 2H), 2.22-2.12 (m, 1H), 2.10-2.01 (m, 3H), 1.97-1.88 (m, 1H), 1.79-1.68 (m, 3H), 1.65-1.55 (m, 3H), 1.47-1.32 (m, 2H), 1.24 (s, 3H), 1.20-1.12 (m, 2H), 1.07-0.97 (m, 2H), 0.88 (d, J=0.9 Hz, 3H). ¹³C NMR (126 MHz, CDCl₃) δ: 217.33, 207.91, 169.12, 143.67, 102.77, 60.00, 50.49, 50.12, 50.06, 48.69, 36.27, 36.03, 35.70, 32.12, 31.24, 31.09, 30.53, 29.02, 28.47, 26.51, 26.18, 26.05, 21.89, 18.40, 14.93. HRMS (ESI): m/z calc. for C₂₅H₃₅NO₃ [M+H]⁺ 398.2617, found: 398.2686. LC-MS Purity (λ: 254 nm): 96.4%.

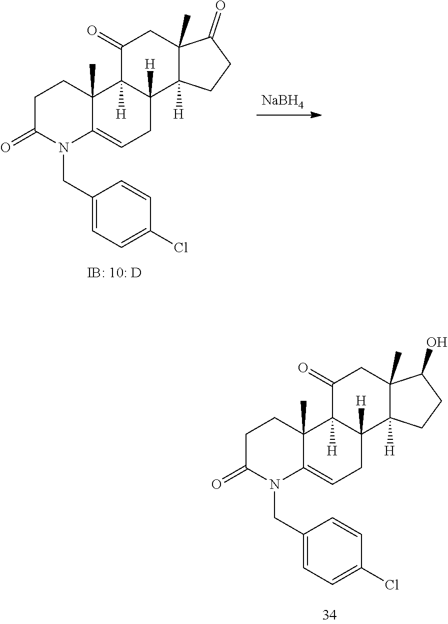

(4aR,4bS,6aS,7S,9aS,9bS)-1-(4-chlorobenzyl)-7-hydroxy-4a,6a-dimethyl-3,4,4a,4b,6,6a, 7,8,9,9a,9b,10-dodecahydro-1H-indeno[5,4-f]quinoline-2,5-dione (compound 34). Substituted enamide starting material (0.368 mmol, 1 eq.) was dissolved in methanol (20 mL) in a round bottom flask and stirred at 0° C. for 10 min. NaBH₄ (0.423 mmol, 1.1 eq) was added, and reaction was removed from ice bath and stirred at room temperature for 1 hour. Reaction was quenched by addition of water, then evaporated under reduced pressure before being transferred to a separatory funnel and extracted with EtOAc (3×). The organic layers were combined, dried with sodium sulfate and concentrated under reduced pressure. The product was purified by silica gel chromatography using 70% EtOAc/Hexanes to 100% EtOAc. Stereochemistry at the C17 position is assumed based on previous reports of identical reaction conditions on structurally analogous compounds. It is also supported by the J>8 Hz coupling present between the corresponding hydrogens on C17 and C16, suggesting pseudo axial-pseudo axial coupling that could only be present in the proposed stereochemical configuration. ¹H NMR (500 MHz, CDCl₃) δ: 7.25 (d, J=8.0 Hz, 2H), 7.07 (d, J=8.1 Hz, 2H), 5.11 (d, J=15.8 Hz, 1H), 4.91 (dd, J=5.8, 2.1 Hz, 1H), 4.62 (d, J=15.9 Hz, 1H), 3.84 (t, J=8.6 Hz, 1H), 2.80-2.65 (m, 2H), 2.65-2.56 (m, 1H), 2.48 (d, J=13.1 Hz, 1H), 2.26-2.12 (m, 3H), 1.97 (d, J=10.7 Hz, 1H), 1.91-1.83 (m, 1H), 1.80-1.69 (m, 2H), 1.63-1.50 (m, 2H), 1.48-1.34 (m, 2H), 1.26-1.20 (m, 4H), 0.72 (s, 3H). ¹³C NMR (126 MHz, CDCl₃) δ: 209.16, 169.22, 143.58, 136.45, 132.65, 128.79, 128.13, 104.40, 79.93, 59.17, 54.66, 49.66, 47.54, 46.50, 35.77, 35.73, 33.17, 31.03, 30.91, 30.66, 28.98, 23.02, 22.82, 17.97, 11.97. HRMS (ESI): m/z calc. for C25H₃₀ClNO₃ [M+H]⁺ 428.1914, found: 428.1991. LC-MS Purity (λ: 254 nm): >99%.

### Example 4. Pharmaceutical Dosage Forms

The following formulations illustrate representative pharmaceutical dosage forms that may be used for the therapeutic or prophylactic administration of a compound of a formula described herein, a compound specifically disclosed herein, or a pharmaceutically acceptable salt or solvate thereof (hereinafter referred to as ‘Compound X’):

| (i) Tablet 1 | mg/tablet |
|---|---|
| ′Compound X′ | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |

| (ii) Tablet 2 | mg/tablet |
|---|---|
| ′Compound X′ | 20.0 |
| Microcrystalline cellulose | 410.0 |
| Starch | 50.0 |
| Sodium starch glycolate | 15.0 |
| Magnesium stearate | 5.0 |
| | 500.0 |

| iii) Capsule | mg/capsule |
|---|---|
| ′Compound X′ | 10.0 |
| Colloidal silicon dioxide | 1.5 |
| Lactose | 465.5 |
| Pregelatinized starch | 120.0 |
| Magnesium stearate | 3.0 |
| | 600.0 |

| (iv) Injection 1 (1 mg/mL) | mg/mL |
|---|---|
| ′Compound X′ (free acid form) | 1.0 |
| Dibasic sodium phosphate | 12.0 |
| Monobasic sodium phosphate | 0.7 |
| Sodium chloride | 4.5 |
| 1.0N Sodium hydroxide solution | q.s. |
| (pH adjustment to 7.0-7.5) | |
| Water for injection | q.s. ad 1 mL |

| (v) Injection 2 (10 mg/mL) | mg/mL |
|---|---|
| ′Compound X′ (free acid form) | 10.0 |
| Monobasic sodium phosphate | 0.3 |
| Dibasic sodium phosphate | 1.1 |
| Polyethylene glycol 400 | 200.0 |
| 0.1N Sodium hydroxide solution | q.s. |
| (pH adjustment to 7.0-7.5) | |
| Water for injection | q.s. ad 1 mL |

| (vi) Aerosol | mg/can |
|---|---|
| ′Compound X′ | 20 |
| Oleic acid | 10 |
| Trichloromonofluoromethane | 5,000 |
| Dichlorodifluoromethane | 10,000 |
| Dichlorotetrafluoroethane | 5,000 |

| (vii) Topical Gel 1 | wt. % |
|---|---|
| ′Compound X′ | 5% |
| Carbomer 934 | 1.25% |
| Triethanolamine | q.s. |
| (pH adjustment to 5-7) | |
| Methyl paraben | 0.2% |
| Purified water | q.s. to 100 g |

| (viii) Topical Gel 2 | wt. % |
|---|---|
| ′Compound X′ | 5% |
| Methylcellulose | 2% |
| Methyl paraben | 0.2% |
| Propyl paraben | 0.02% |
| Purified water | q.s. to 100 g |

| (ix) Topical Ointment | wt. % |
|---|---|
| ′Compound X′ | 5% |
| Propylene glycol | 1% |
| Anhydrous ointment base | 40% |
| Polysorbate 80 | 2% |
| Methyl paraben | 0.2% |
| Purified water | q.s. to 100 g |

| (x) Topical Cream 1 | wt. % |
|---|---|
| ′Compound X′ | 5% |
| White bees wax | 10% |
| Liquid paraffin | 30% |
| Benzyl alcohol | 5% |
| Purified water | q.s. to 100 g |

| (xi) Topical Cream 2 | wt. % |
|---|---|
| ′Compound X′ | 5% |
| Stearic acid | 10% |
| Glyceryl monostearate | 3% |
| Polyoxyethylene stearyl ether | 3% |
| Sorbitol | 5% |
| Isopropyl palmitate | 2% |
| Methyl Paraben | 0.2% |
| Purified water | q.s. to 100 g |

These formulations may be prepared by conventional procedures well known in the pharmaceutical art. It will be appreciated that the above pharmaceutical compositions may be varied according to well-known pharmaceutical techniques to accommodate differing amounts and types of active ingredient ‘Compound X’. Aerosol formulation (vi) may be used in conjunction with a standard, metered dose aerosol dispenser. Additionally, the specific ingredients and proportions are for illustrative purposes. Ingredients may be exchanged for suitable equivalents and proportions may be varied, according to the desired properties of the dosage form of interest.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

All publications, patents, and patent documents are incorporated by reference herein, as though individually incorporated by reference. No limitations inconsistent with this disclosure are to be understood therefrom. The invention has been described with reference to various specific and preferred embodiments and techniques. However, it should be understood that many variations and modifications may be made while remaining within the spirit and scope of the invention.

1. A compound of Formula I: 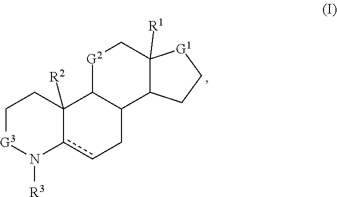
or a salt thereof;
wherein [symbol]
is an unsaturated or saturated bond;
G¹ is C═O, CHF, CF₂, CHJ¹R^{a}, or C(OCH₂)₂;
J¹ is O, S, NR^{c}, C(O)NR^{d};
G² is C═O, CH₂, CHF, CF₂, CHJ²R^{b}, or C(OCH₂)₂;
J² is O, S, NR^{g}, C(O)X² wherein X² is O or NR^{h};
G³ is C═O or CH₂;
R^{a}, R^{b}, R^{c}, R^{d}, R^{g}, and R^{h} are each independently H, or -(C₁-C₆)alkyl;
R¹ is -(C₁-C₆)alkyl;
R² is -(C₁-C₆)alkyl;
R³ is —CH₂R⁴, —CH(CH₃)R⁴, R⁴, or —C(O)R⁴ wherein R³ is not —C(O)R⁴ when G³ is C═O; and
R⁴ is aryl, heteroaryl, cycloalkyl, or heterocyclyl, wherein aryl and heteroaryl are each optionally substituted with one or more substituents;
wherein the compound is not (4aR,4bS,6aS,9aS,9bS)-1-(4-chlorobenzyl)-4a,6a-dimethyl-3,4,4a,6,6a,8,9,9a,9b,10-decahydro-1H-indeno[5,4-f]quinoline-2,5,7(4bH)-trione or (4aR,4bS,6aS,9aS,9bS)-1-benzyl-4a,6a-dimethyl-3,4,4a,6,6a,8,9,9a,9b,10-decahydro-1H-indeno[5,4-f]quinoline-2,5,7(4bH)-trione.

2. The compound of claim 1 wherein R⁴ is: 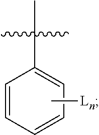
wherein
each Lₙ is independently halo, X³R^{e} or -(C₁-C₆)alkyl;
n is 1, 2, 3, 4, 5, or 0;
each X³ is independently O, S, or NR^{f}; and
R^{e} and R^{f} are each independently H or -(C₁-C₆)alkyl.

3. The compound of claim 2 wherein one L is in the para-position.

4. The compound of claim 1 wherein G¹ is C═O or CHOH.

5. The compound of claim 1 wherein G² and G³ are C═O.

6. The compound of claim 1 wherein R¹ and R² are CH₃.

7. The compound of claim 1 wherein R³ is: 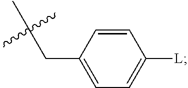
wherein
L is halo, X³R^{e}, or -(C₁-C₆)alkyl;
X³ is O, S, or NR^{f}; and
R^{e} and R^{f} are H or -(C₁-C₆)alkyl.

8. The compound of claim 1 represented by Formula II: 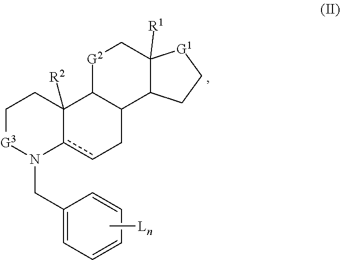
or a pharmaceutically acceptable salt thereof;
wherein
G¹ is C═O or CHJ¹R^{a};
G² is C═O or CHJ²R^{b};
each Lₙ is each independently halo, X³R^{e} or -(C₁-C₆)alkyl;
n is 1 or 2;
each X³ is each independently O, S, or NR^{f}; and
R^{e} and R^{f} are each independently H or -(C₁-C₆)alkyl;
wherein each -(C₁-C₆)alkyl is optionally substituted with one or more substituents.

9. The compound of claim 1 represented by Formula III: 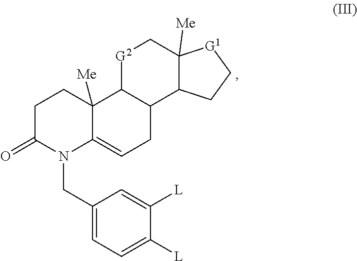
or a pharmaceutically acceptable salt thereof;
wherein
G¹ is C═O or CHJ¹R^{a};
G² is C═O or CHJ²R^{b};
each L is independently halo, X³R^{e}, -(C₁-C₆)alkyl, or H;
each X³ is independently O, S, or NR^{f}; and
R^{e} and R^{f} are each independently H or -(C₁-C₆)alkyl.

10. The compound of claim 1 represented by Formula IV: 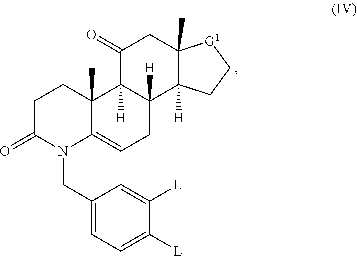
or a pharmaceutically acceptable salt thereof;
wherein
each L is independently halo, X³R^{e}, -(C₁-C₆)alkyl, or H;
each X³ is independently O, S, or NR^{f}; and
R^{e} and R^{f} are each independently H or -(C₁-C₆)alkyl.

11. The compound of claim 1 represented by Formula V: 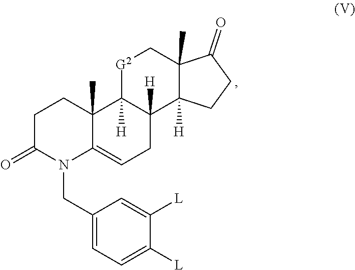
or a pharmaceutically acceptable salt thereof;
wherein
each L is independently halo, X³R^{e}, -(C₁-C₆)alkyl, or H;
each X³ is independently O, S, or NR^{f}; and
R^{e} and R^{f} are each independently H or -(C₁-C₆)alkyl.

12. The compound of claim 1 wherein the compound is (3):

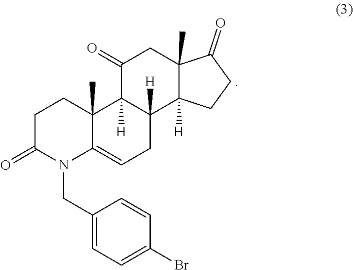

13. The compound of claim 1 wherein the compound is:

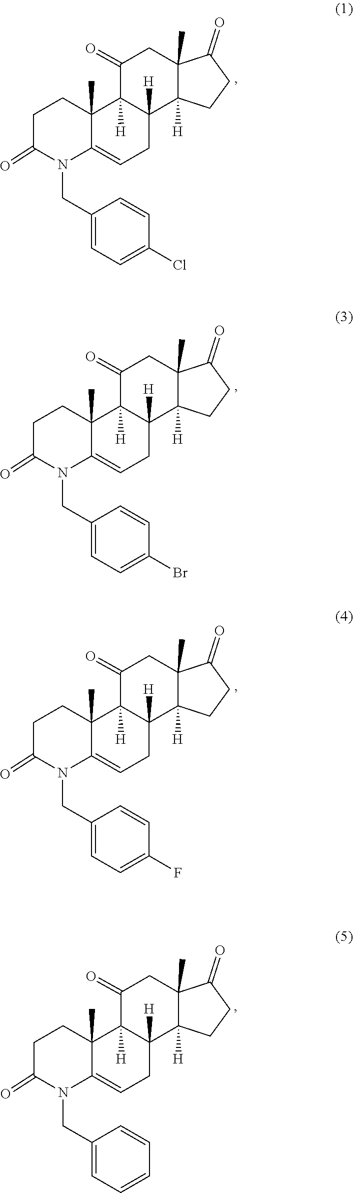

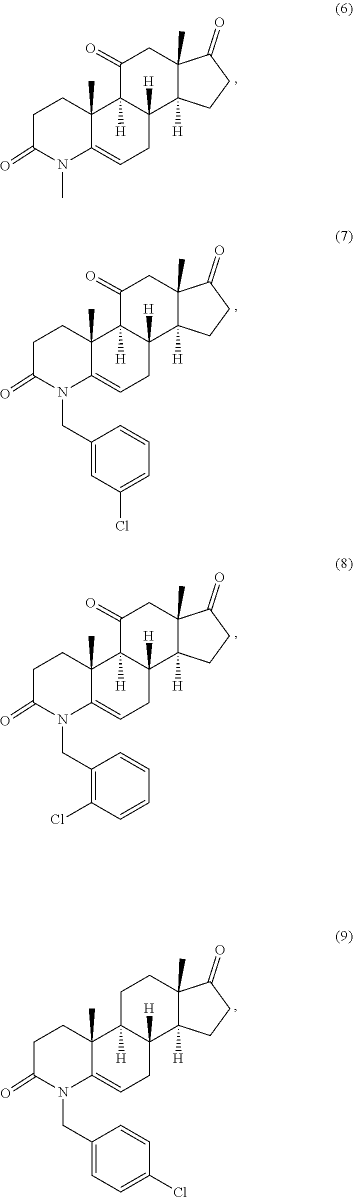

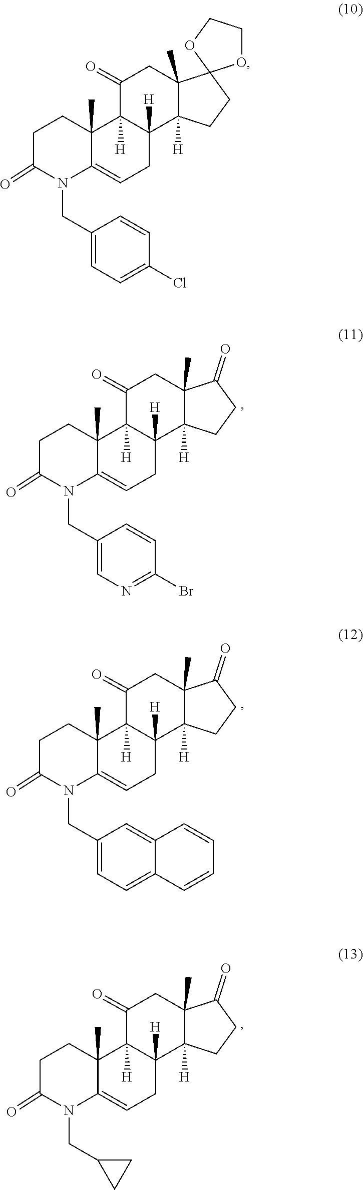

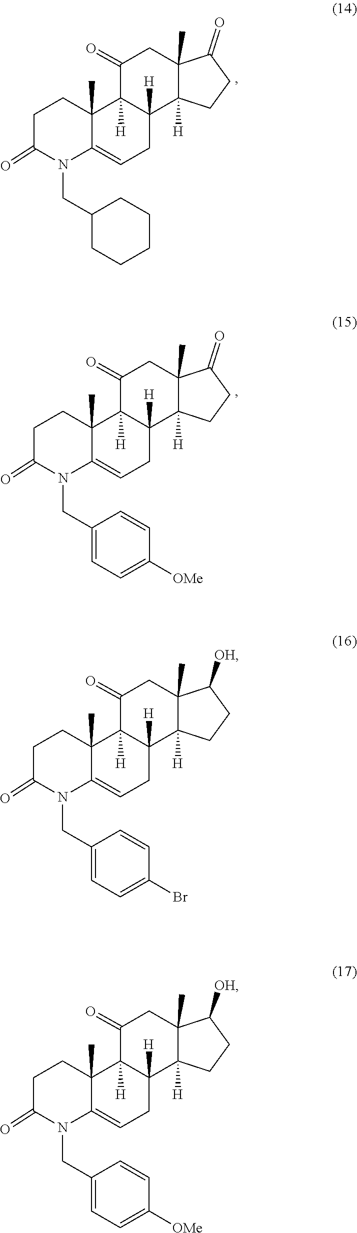

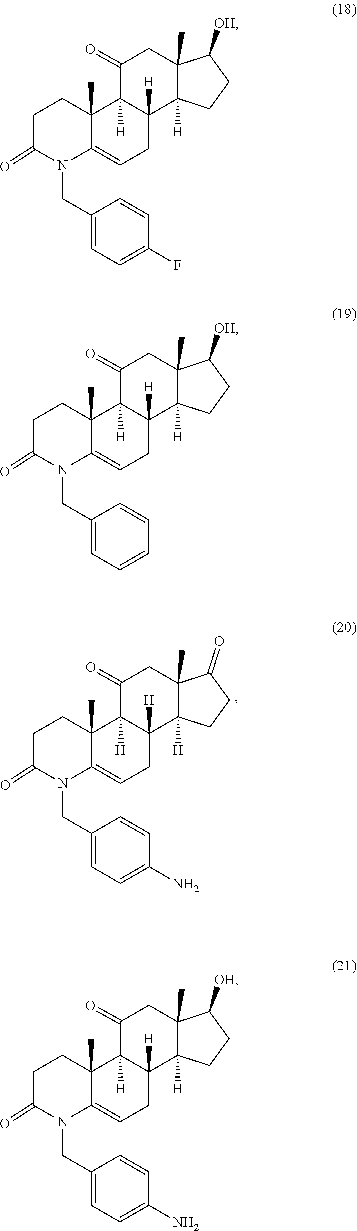

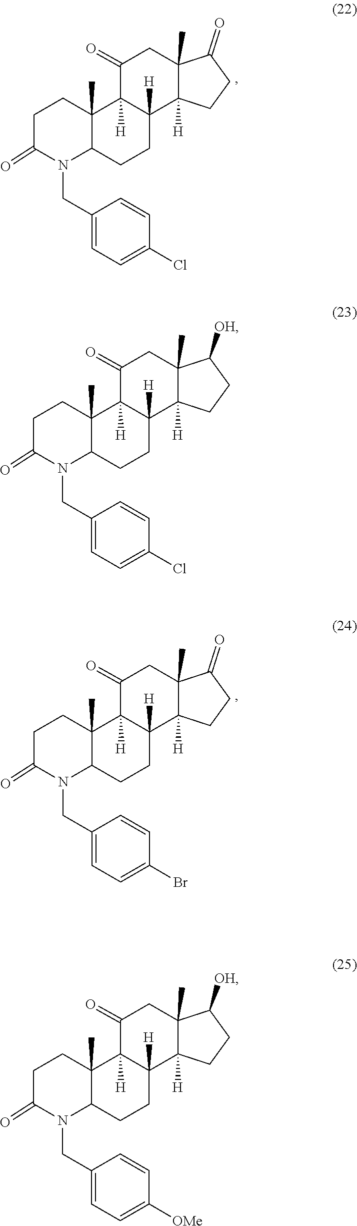

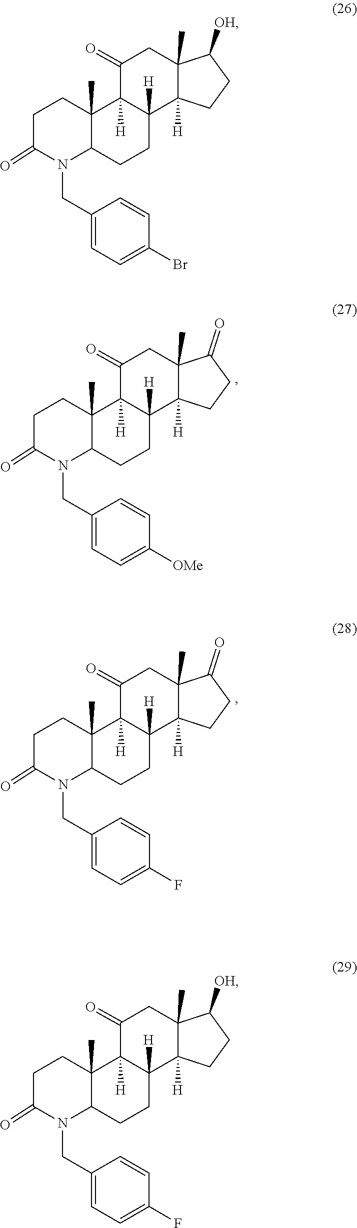

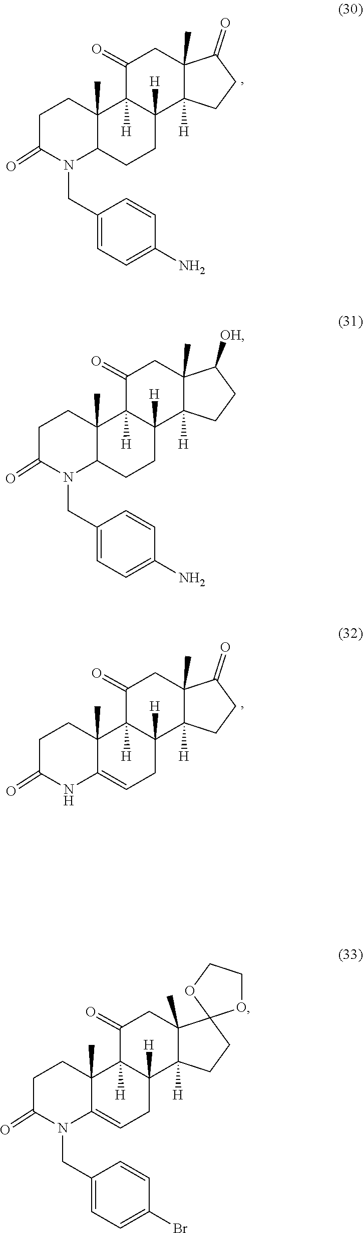

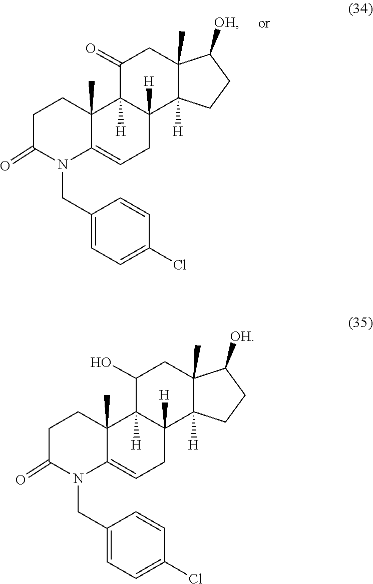

14. A composition comprising a compound of claim 1 and a pharmaceutically acceptable excipient.

15. A method for treatment of cancer comprising, administering to a subject in need of cancer treatment a compound of Formula I: 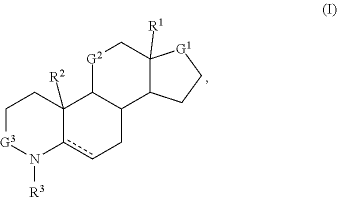
or a pharmaceutically acceptable salt thereof;
wherein [symbol]
is an unsaturated or saturated bond;
G¹ is C═O, CH₂, CHF, CF₂, CHJ¹R^{a}, or C(OCH₂)₂;
J¹ is O, S, NR^{c}, C(O)X¹ wherein X¹ is O or NR^{d};
G² is C═O, CH₂, CHF, CF₂, CHJ²R^{b}, or C(OCH₂)₂;
J² is O, S, NR^{g}, C(O)X² wherein X² is 0 or NR^{h};
G³ is C═O or CH₂;
R^{a}, R^{b}, R^{c}, R^{d}, R^{g}, and R^{h} are each independently H, or -(C₁-C₆)alkyl;
R¹ is -(C₁-C₆)alkyl;
R² is -(C₁-C₆)alkyl;
R³ is —CH₂R⁴, —CH(CH₃)R⁴, R⁴, —C(O)R⁴, or H wherein R³ is not —C(O)R⁴ when G³ is C═O; and
R⁴ is aryl, heteroaryl, cycloalkyl, heterocyclyl, or -(C₁-C₆)alkyl, wherein aryl, heteroaryl, cycloalkyl, and heterocyclyl are each optionally substituted with one or more substituents;
wherein each -(C₁-C₆)alkyl is independently saturated or unsaturated, and optionally substituted with one or more substituents;
wherein the compound increases T-cell activity via inhibition of the enzyme SULT2B1b, wherein immune clearance of the cancer increases in the subject, thereby treating the cancer.

16. The method of claim 15 wherein R⁴ is: 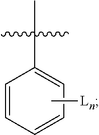
wherein
each Lₙ is independently halo, X³R^{e} or -(C₁-C₆)alkyl;
n is 1, 2, 3, 4, 5, or 0;
each X³ is independently O, S, or NR^{f}; and
R^{e} and R^{f} are each independently H or -(C₁-C₆)alkyl.

17. The method of claim 15 wherein the compound and a second agent are simultaneously or sequentially administered to the subject for the treatment of the cancer.

18. (canceled)

19. (canceled)

20. (canceled)

21. (canceled)

22. The method of claim 15 wherein the compound is (4aR,4bS,6aS,9aS,9bS)-1-(4-bromobenzyl)-4a,6a-dimethyl-3,4,4a,6,6a,8,9,9a,9b,10-decahydro-1H-indeno[5,4-f]quinoline-2,5,7(4bH)-trione (3) or (4aR,4bS,6aS,7S,9aS,9bS)-1-(4-bromobenzyl)-7-hydroxy-4a,6a-dimethyl-3,4,4a,4b,6,6a,7,8,9,9a,9b,10-dodecahydro-1H-indeno[5,4-f]quinoline-2,5-dione (34).

23. A method for in-vivo inhibition of an enzyme comprising contacting a compound of claim 1 and the enzyme sulfotransferase family cytosolic 2B member 1 (SULT2B1b) wherein the in-vivo inhibition of the enzyme suppresses production of cholesterol sulfate.

24. A method for inducing death of cancer cells comprising contacting a compound of claim 1 and the cancer cells wherein the compound initiates an immune response by lowering levels of cholesterol sulfate via inhibition of the enzyme SULT2B1b, thereby inducing an immune response mediated death of the cancer cells.
